(19) United States
(12) Patent Application Publication
(10) Pub. No.: US 2024/0307363 A1
(43) Pub. Date: Sep. 19, 2024

(54) METHODS FOR TREATING A PULMONARY DISEASE WITH AN ALK-5 (TGF BETA R1) INHIBITOR

(71) Applicant: Thirona Bio, Inc., San Diego, CA (US)

(72) Inventors: David A. BULLOUGH, Encinitas, CA (US); John Gordon FOULKES, Rancho Santa Fe, CA (US); Nigel R.A. Beeley, Sturgeon Bay, WI (US); Roger Crystal, Pacific Palisades, CA (US)

(73) Assignee: Thirona Bio, Inc., San Diego, CA (US)

(21) Appl. No.: 18/557,253

(22) PCT Filed: May 3, 2022
(86) PCT No.: PCT/US2022/027402

§ 371 Date: Oct. 25, 2023

Related U.S. Application Data
(60) Provisional application No. 63/183,393, filed on May 3, 2021.

(51) Int. Cl. A61K 31/444; A61K 9/00; A61P 11/00; A61P 35/00

(52) U.S. Cl. CPC A61K 31/444; A61K 9/0073; A61P 11/00; A61P 35/00

(57) ABSTRACT

The present invention concerns liquid, dry powder and metered-dose formulations for therapeutic inhaled delivery of ALK5 (TGF-βR1) inhibitor containing compositions to desired anatomical sites, for treatment or prophylaxis of a variety of pulmonary disease conditions such as Idiopathic Pulmonary Fibrosis, Idiopathic Interstitial Pneumonia, scleroderma-associated interstitial lung disease, sarcoidosis, cystic fibrosis, lung cancer, and a COVID infection.

This application claims priority of U.S. Provisional Application No. 63/183,393, filed May 3, 2021, the contents of which are hereby incorporated by reference.

### TECHNICAL FIELD

This invention concerns liquid, dry powder and metered-dose formulations for inhaled delivery of compositions comprising an ALK5 (TGF-βR1) inhibitor to a desired anatomical site, for treatment or prophylaxis of a variety of pulmonary diseases.

### BACKGROUND

A number of pulmonary diseases such as lung fibrosis, chronic obstructive pulmonary disease (COPD; and sub-class diseases therein), asthma and cystic fibrosis are initiated by an external challenge. As non-limiting examples, these initiators include infection, cigarette smoking, environmental exposure, radiation exposure, a surgical procedure and transplant rejection. Other causes include genetic predisposition and the effects of aging. Recent publications have also raised the concern that infection by COVID-19 or one of its variants may lead to lung fibrosis.

Idiopathic pulmonary fibrosis (IPF) is the most common type of idiopathic interstitial pneumonia and is characterized by a poor prognosis, with an estimated 5-year chance of survival of approximately 20%. Progressive and irreversible lung functional impairment leads to chronic respiratory insufficiency with a severely impaired quality of life. In IPF, injured dysfunctional alveolar epithelial cells promote fibroblast recruitment and proliferation, resulting in scarring of lung tissue. In the last 2 decades, novel treatments for IPF have been developed as a consequence of an increasing understanding of disease pathogenesis and pathobiology. However, one of the major problems in developing effective treatments for IPF is the redundancy of the pathways involved in its pathogenesis. Similar to cancer, inhibiting single mediators or signaling pathways is largely ineffective in reducing the progression of IPF. Thus, the two approved drugs for IPF, nintedanib and pirfenidone, do not reverse or cure the disease, but only slow disease progression.

Aberrant signaling by Transforming Growth Factor-β (TGF-β) and its type I (ALK5) receptor has been implicated in multiple human diseases and this pathway is considered a potential major target for treating pulmonary fibrosis. Indeed, TGF-β has been called the “Titan of Lung Fibrosis” (Yue et al (2010) Curr. Enzyme Inhib. 6(2): 10.2174/10067. Furthermore, over 30 years of research, and thousands of publications, have demonstrated the central role of TGF-β in almost every fibrotic disease.

Previous efforts to inhibit the TGF-β pathway have focused on achieving systemic exposure of a therapeutic amount of an agent targeting either TGF-β ligands or the kinase activity of the TGF-β receptor, ALK5. However, despite significant effort by multiple pharmaceutical companies to develop such inhibitors, successful progression through clinical trials has proven difficult. Given the pleiotropic biology of TGF-β, finding an acceptable therapeutic window represents a major challenge when using systemic dosing with a TGF-β inhibitor. Inhalation therapy by administering ALK5 inhibitors into the lung, has the advantage of producing the greatest local effect with less potential systemic toxicity. That is, improving the ratio of therapeutic efficacy to adverse side effects.

Described herein are compositions of ALK5 (TGF-βR1) inhibitor compounds suitable for inhalation delivery to the lungs and/or to a systemic compartment and methods of using such compositions.

The treatment of lung diseases by means of aerosols or other inhalable delivery vehicles allow a targeted pharmaceutical therapy because the active agent can be delivered directly to the pharmacological target site by means of inhalation devices. This requires that the inhaled droplets or particles reach the target tissue and are deposited there. In general, the smaller the diameter of the aerosol particles, the greater the probability that active agents reach the peripheral parts of the lungs. Depending on the kind and extent of drug particle deposition, diseases such as lung fibrosis, asthma, chronic obstructive pulmonary disease (COPD) and pulmonary emphysema can be treated “quasi-topically” by inhalation. At present, multiple methods are used for the administration of active agents by inhalation. These methods include pressurized gas propelled metered dose inhalers, powder inhalers and nebulizers. The type and extent of deposition at the target site depends on the droplet or particle size, the anatomy of the respiratory tract of the human patient being treated and the overall functional capacity of the diseased lung.

### SUMMARY

The present invention provides an ALK5 (TGF-βR1) inhibitor compound formulation or composition suitable for oral pulmonary or intranasal inhalation delivery comprising a formulation suitable for aerosol administration of an ALK5 inhibitor compound for use in the prevention or treatment of various fibrotic and inflammatory diseases associated with the lung,

Some embodiments disclosed herein provide a method of treating a lung disease in a mammalian subject comprising administering an ALK5 inhibitor compound, wherein the compound or pharmaceutically acceptable salt thereof is administered as an aerosol to the mammal via oral pulmonary or intranasal inhalation delivery.

Other embodiments disclosed herein provide a method of treating lung disease in a mammal comprising administering an ALK5 inhibitor compound, wherein the compound or pharmaceutically acceptable salt thereof is administered as a dry powder to a mammalian subject via oral pulmonary or intranasal inhalation delivery.

One embodiment disclosed herein involves administering an ALK5 inhibitor compound having structural Formula I:

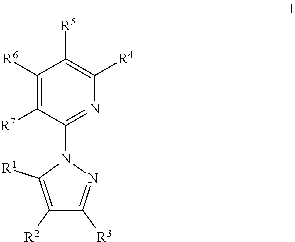

as well as prodrugs and pharmaceutically acceptable salts thereof.

In some embodiments of Formula (I):
R¹ is selected from the group consisting of thieno[3,2-c]pyridinyl, thieno[3,2-b]pyridinyl, thieno[2,3-c]pyridinyl, and thieno[2,3-b]pyridinyl; wherein each group may be optionally substituted with one to three substituents independently selected from the group consisting of C₁-C₃-alkyl, —(C₁-C₃-alkyl)S(C₁-C₃-alkyl), —S(C₁-C₃-alkyl), —(C₁-C₃-alkyl)O(C₁-C₃-alkyl), —O(C₁-C₃-alkyl), —C(═O)O(C₁-C₃-alkyl), —CO₂H, —C(—O)NR⁸R⁹, halo, —CN, and —OH;
R² and R³ are independently selected from the group consisting of H, C₁-C₃-alkyl, —(C₁-C₃-alkyl)S(C₁-C₃-alkyl), —S(C₁-C₃-alkyl), —(C₁-C₃-alkyl)O(C₁-C₃-alkyl), —O(C₁-C₃-alkyl), —C(═O)O(C₁-C₃-alkyl), —CO₂H, —C(═O)NR¹⁰R¹¹, halo, —CN, —OH, and C₃-C₆-cycloalkyl;
alternatively, R² and R³ may together form a 5-6-membered heteroaryl, phenyl, a C₄-C₆-cycloalkyl, or a 4-6-membered heterocycloalkyl; wherein C₄-C₆-cycloalkyl and 4-6-membered heterocycloalkyl may be optionally substituted with one to three substituents independently selected from the group consisting of halo, —OH, oxo, and C₁-C₃ alkyl; and wherein 5-6-membered heteroaryl and phenyl may be optionally substituted with one to three substituents independently selected from the group consisting of halo, —CN, —OH, —O(C₁-C₃ alkyl), and C₁-C₃ alkyl;
R⁴, R⁵, R⁶, and R⁷ are selected from the group consisting of H, C₃-cycloalkyl, C₁-C₃-alkyl, —(C₁-C₃-alkyl)S(C₁-C₃-alkyl), —S(C₁-C₃-alkyl), —(C₁-C₃-alkyl)O(C₁-C₃-alkyl), —O(C₁-C₃-alkyl), —C(═O)O(C₁-C₃-alkyl), —CO₂H, —C(═O)NR¹²R¹³, halo, —CN, —OH;
R⁸ and R⁹ are each independently selected from the group consisting of H, and —(C₁-C₃ alkyl)OH, C₁-C₃-alkyl, halo, and —O(C₁-C₃-alkyl);
R¹⁰ and R¹¹ are each independently selected from the group consisting of H and C₁-C₃ alkyl; and
R¹² and R¹³ are each independently selected from the group consisting of H, C₁-C₃ alkyl, halo, and —O(C₁-C₃-alkyl).

One embodiment disclosed herein includes administering an ALK5 inhibitor compound having the structure of Formula II:

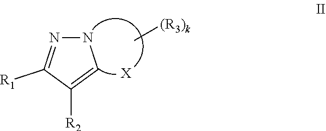

as well as prodrugs and pharmaceutically acceptable salts thereof.

One embodiment disclosed herein includes administering an ALK5 inhibitor compound having the structure of Formula III:

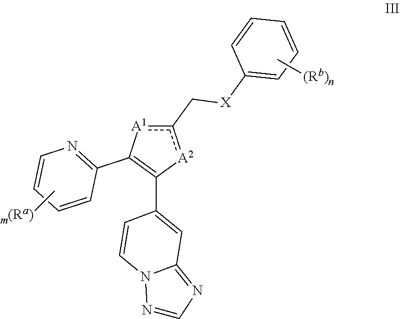

as well as prodrugs and pharmaceutically acceptable salts thereof.

One embodiment disclosed herein includes administering an ALK5 inhibitor compound having the structure of Formula IV:

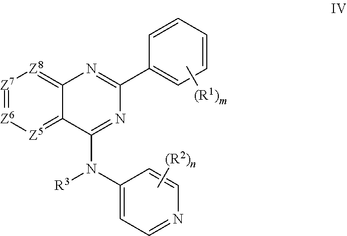

as well as prodrugs and pharmaceutically acceptable salts thereof.

One embodiment disclosed herein includes administering an ALK5 inhibitor compound having the structure of Formula V:

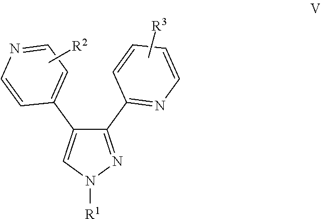

as well as prodrugs and pharmaceutically acceptable salts thereof.

One embodiment disclosed herein includes administering an ALK5 inhibitor compound having the structure of Formula VI:

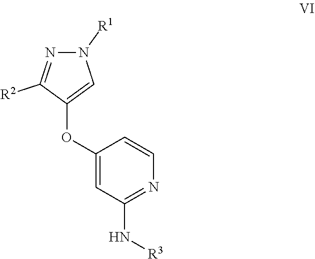

as well as prodrugs and pharmaceutically acceptable salts thereof.

One embodiment disclosed herein includes administering an ALK5 inhibitor compound having the structure of Formula VII:

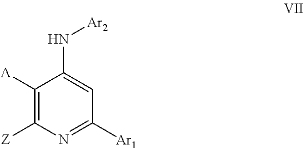

as well as prodrugs and pharmaceutically acceptable salts thereof.

One embodiment disclosed herein includes administering a compound having the structure of

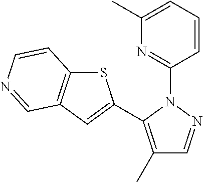 [SMILES: Cc1cccc(-n2ncc(C)c2-c2cc3cnccc3s2)n1]

In some embodiments, the present disclosure is a soft ALK5 inhibitor. As used herein, the term “soft drug” or “soft ALK5 inhibitor” refers to a biologically active compound that is converted upon entering the systemic circulation into a predictable metabolite that exhibits reduced biological activity relative to the parent compound. A soft drug preferably exerts its desired therapeutic effect locally at the target organ or tissue, then is rapidly converted to a less active metabolite upon entering the systemic circulation, thus reducing systemic exposure to the biologically active compound. Accordingly, soft drugs have a lower potential for undesired side effects relative to non-soft drug compounds having comparable biological activity. Preferably, a soft drug of the present disclosure exhibits good stability at the intended site of action (e.g., the lung), and is rapidly metabolized upon entering systemic circulation

In some embodiments, the present disclosure is a soft ALK5 inhibitor that is oxidized and is thereby rapidly metabolized in the liver. Preferably, the soft drug is a potent inhibitor of ALK5 activity, while the corresponding oxidized soft drug exhibits reduced ALK5 inhibitory activity. For example, the difference in inhibitory potency and the ALK5 inhibitor and the corresponding oxidized ALK5 inhibitor may be 10 to 100-fold. In some embodiments, a soft ALK5 inhibitor of the present disclosure is administered to the lung, for example, by inhalation, and inhibits the activity of ALK5 in the lung. However, upon exiting the lung, the soft ALK5 inhibitor may be readily oxidized in the liver, thus reducing systemic exposure to the soft drug.

Another embodiment disclosed herein include administering the compound of Formulas I, II, III, IV, V, VI, and VII with a nebulizer, a metered dose inhaler, or a dry powder inhaler.

Other embodiments disclosed herein include liquid or dry powder formulations of a compound of Formulas I, II, III, IV, V, VI, and VII.

Other embodiments disclosed herein include administering the compound of Formulas I, II, III, IV, V, VI, and VII at least once a week, on a continuous daily dosing schedule, once a day, twice a day, or three times a day.

Non-limiting examples of diseases which can be treated with the compounds and compositions provided herein include a variety of lung cancers as well as all types of pulmonary fibrosis. Pulmonary diseases such as interstitial lung disease including Idiopathic Pulmonary Fibrosis (IPF), Idiopathic Interstitial Pneumonia (IIP), scleroderma-associated interstitial lung disease (SSc-ILD), sarcoidosis, bronchiolitis obliterans, Langerhans cell histiocytosis (also called Eosinophilic granuloma or Histiocytosis X), chronic eosinophilic pneumonia, collagen vascular disease, granulomatous vasculitis, Goodpasture's syndrome, pulmonary alveolar proteinosis (PAP), and cystic fibrosis.

One embodiment includes a method for treating idiopathic pulmonary fibrosis (IPF) with a compound of Formulas I, II, III, IV, V, VI, and VII.

One embodiment includes a method for treating scleroderma-associated interstitial lung disease (SSc-ILD) with a compound of Formulas I, II, III, IV, V, VI, and VII.

It is to be understood that both the foregoing general description and the following detailed description are exemplary and explanatory only and are not restrictive of the disclosure, as claimed.

### DETAILED DESCRIPTION

Provided herein are compositions and methods for the prevention or treatment of various fibrotic and inflammatory diseases associated with the lung, by methods such as oral pulmonary or intranasal inhalation delivery of aerosolized ALK5 (TGF-βR1) inhibitor compounds.

A number of undesirable pulmonary diseases such as interstitial lung disease (ILD; and sub-class diseases therein), chronic obstructive pulmonary disease (COPD; and sub-class diseases therein), asthma, cystic fibrosis, and fibrotic indications of the lungs, are initiated from an external challenge. By non-limiting example, these effectors can include infection, cigarette smoking, environmental exposure, radiation exposure, surgical procedures and transplant rejection. However, other causes related to genetic disposition and the effects of aging may also be attributed.

In epithelium, scarring serves a valuable healing role following injury. However, epithelium tissue may become progressively scarred following more chronic and or repeated injuries resulting in abnormal function. In the case of idiopathic pulmonary fibrosis (IPF; and other subclasses of ILD) and cystic fibrosis, if a sufficient proportion of the lung becomes scarred respiratory failure can occur. In any case, progressive scarring may result from a recurrent series of insults to different regions of the organ or a failure to halt the repair process after the injury has healed. In such cases the scarring process becomes uncontrolled and deregulated. In some forms of fibrotic disease, scarring remains localized to a limited region, but in others it can affect a more diffuse and extensive area resulting in direct or associated organ failure.

In conditions such as pulmonary fibrosis, physiological responses characterized by control of pro-inflammatory and pro-fibrotic factors with an ALK5 inhibitor compound may be beneficial to attenuate and/or reverse fibrosis. Therapeutic strategies exploiting such ALK5 inhibitor compounds effects in these diseases and other indications are contemplated herein.

Transforming growth factor-β (TGF-β) is a secreted protein found in three distinct isoforms, TGF-β1, TGF-β2, and TGF-β3 (ten Dijke et al 1988 Proc. Nat. Acad. Sci. 85, pp. 4715-4719: Tzavlaki & Moustakas, Biomolecules 2020, 10, 487). The three TGF-βs share over 80% sequence identity, bind to the same receptor system, and utilize the same signal transduction mechanisms. However, they have different promoter regions and show cell type specific expression. While multiple pathways participate in the complex TGF-β signaling process, in the simplest model, TGF-β binding to its receptor activates the ALK-5 kinase domain, which phosphorylates the SMAD transcription factors (amongst others), which in turn upregulate a pro-fibrotic response involving multiple genes (Walton et al 2017 Front. Pharmacol.; 8: 461). There are also SMAD independent TGF-β signaling process, but they too are controlled by the ALK-5 kinase domain (Liu et al 2017, Exper & Therap Med. 13, 2123-2128). Thus, inhibiting the ALK-5 kinase has the potential to block excess fibrosis driven by this mechanism.

TGF-β is an evolutionarily conserved pleiotropic factor that regulates a myriad of biological processes including development, tissue regeneration, immune responses, and tumorigenesis. TGF-β is necessary for lung organogenesis and homeostasis as evidenced by genetically engineered mouse models. TGF-β is crucial for epithelial-mesenchymal interactions during lung branching morphogenesis and alveolarization. Expression and activation of the three TGF-β ligand isoforms in the lungs are temporally and spatially regulated by multiple mechanisms. The lungs are structurally exposed to extrinsic stimuli and pathogens, and are susceptible to inflammation, allergic reactions, and carcinogenesis. Upregulation of TGF-β ligands is observed in major pulmonary diseases, including pulmonary fibrosis, emphysema, bronchial asthma, and lung cancer. TGF-β regulates multiple cellular processes such as growth suppression of epithelial cells, alveolar epithelial cell differentiation, fibroblast activation, and extracellular matrix organization. These effects are closely associated with tissue remodeling in pulmonary fibrosis and emphysema. TGF-β is also central to T cell homeostasis and is deeply involved in asthmatic airway inflammation. TGF-β is the most potent inducer of epithelial-mesenchymal transition in non-small cell lung cancer cells and is pivotal to the development of tumor-promoting microenvironment in the lung cancer tissue (Noguchi et al (2018) Inter. J. Molecular Sciences, 19(8), 3674).

TGF-β signaling has also been implicated in the pathogenesis of asthma and chronic obstructive pulmonary disease (COPD). Both diseases are characterized by airway obstruction (typically considered reversible in asthma but irreversible in COPD), inflammation, and remodeling. TGF-1 levels were elevated in bronchoalveolar lavage (BAL) fluid obtained from asthmatic patients and in the airway and alveolar epithelium of patients with COPD. Higher producing polymorphisms in TGF-β1 are associated with worsening asthma severity. Mechanistically, it is hypothesized that TGF-β produces pathologic effects in these diseases by promoting goblet cell hyperplasia, subepithelial fibrosis, epithelial damage, and airway smooth muscle hypertrophy.

TGF-β signaling in certain contexts also drives a number of processes involved in cystic fibrosis (CF) lung disease pathophysiology, including fibrosis, goblet cell hyperplasia, abnormal inflammatory responses, and dysregulated ion transport (Kramer et al., 2018 Expert Opinion on Therapeutic Targets, 22(2), 177-189). TGF-β downregulates epithelial chloride transport to exacerbate already dysregulated ion transport in epithelia throughout the body in CF and drives goblet cell hyperplasia and mucin secretion, which are pathologic features of CF lung disease. In addition, TGF-β leads to aberrant inflammatory responses and GF lungs are known to be compromised for a more aggressive inflammatory response, inability to clear chronic infection, and dysregulated innate immunity. Finally, TGF-β promotes fibrosis and, after cycles of infection and inflammation, fibrotic lung disease, significantly contributes to pulmonary decline in CF patients.

TGF-β may be important in both early and late CF disease (Kramer et al (2018) American Journal of Physiology, 315(3), L456-L465). Native latent TGF-β expression was induced after treatment with Ad-TGF-β, suggesting that positive feedback may occur, which may be relevant in early CF disease. In addition, TGF-β has also been implicated in later pulmonary fibrosis and airway remodeling in CF, through driving myofibroblast differentiation and proliferation. Studies of lung specimens obtained from CF patients have identified TGF-β signaling associated with regions of intense fibrosis and myofibroblast proliferation and CF adolescents with refractory lung function decline were found to have constrictive bronchiolitis, fibrogenesis, and increased TGF-β signaling. These data highlight a potential role for TGF-β in driving the irreversible airway remodeling and fibrosis that contributes to pulmonary morbidity and mortality in CF.

TGF-β is also a genetic modifier of CF lung disease (Kramer et al. 2018 Expert Opinion on Therapeutic Targets, 22(2), 177-189). Two TGF-β1 polymorphisms (C-509T polymorphism in the promotor region and T29C polymorphism in codon 10) have been found in genome-wide association studies to be linked to more severe cystic fibrosis (CF) lung disease. Furthermore, the levels of TGF-β1 were elevated in plasma and bronchoalveolar lavage fluid from CF patients and were associated with reduced pulmonary function. Increased levels of TGF-β1 in the serum of CF patients with exacerbation as well as non-exacerbation when compared to healthy controls: the levels were significantly higher in exacerbation phase. Studies have also reported increased levels of TGF-β1 in all types of bacterial infections, while the increase in levels were more in patients infected with Pseudomonas aeruginosa. These polymorphisms have been linked in vitro and in non-CF populations to higher levels of gene expression and secretion. Transfection of the T29C polymorphism in Hela cells causes a 2.8-fold increase in TGF-β1 secretion and having at least a single copy of the T29C allele was associated with increased serum TGF-β1 protein levels. The C-509T promotor region polymorphism is associated with higher transcriptional activity of TGF-β1 in vitro and higher plasma TGF-β1 levels. However, the relationship between these polymorphisms and blood or BAL levels of TGF-β1 has not been clearly defined in the CF population.

TGF-β's role is also a biomarker of increased lung disease severity in CF. Increased TGF-β1 blood (plasma) and BAL levels in CF patients have been associated with pulmonary exacerbations, severity of lung disease, increased neutrophilic inflammation in BAL, infection with Pseudomonas aeruginosa, and several clinical phenotypes of CF (Sagwal et al., 2020 Lung, 198(2), 377-383). Taken together, these studies implicate TGF-β1 as a prominent potential therapeutic target in CF.

The burden of fibrotic lung disease following SARS-CoV-2 infection is expected to increase significantly given the global scale of the pandemic. The most severe cases of COVID-19 have extensive pulmonary fibrotic tissue, and serum levels of cytokines and growth factors including TGF-β are strongly increased. Sudden and uncontrolled increases in active TGF-β, along with other pro-inflammatory cytokines such as TNF-α, IL-6, and IL-1β (the “cytokine storm”) inevitably result in rapid and massive edema and fibrosis that remodels and blocks the airways (George et al 2020. Lancet Respiratory Medicine, 8(8), 807-815).

It is believed that because available anti-fibrotic therapies have broad anti-fibrotic activity regardless of disease etiology, that these drugs might have a role in attenuating pro-fibrotic pathways in SARS-CoV-2 infection. Indeed, recent articles suggest that agents like pirfenidone and nintedanib should be tested in COVID-19 patients (George et al., 2020 Lancet Respiratory Medicine, 8(8), 807-815). However, pirfenidone and nintedanib are commercially available only in oral form and so they cannot be used in patients who are intubated and mechanically ventilated.

Persistent post-COVID syndrome, also referred to as long COVID, is a pathologic entity, which involves persistent physical, medical, and cognitive sequelae following COVID-19, including persistent immunosuppression as well as pulmonary, cardiac, and vascular fibrosis. Pathologic fibrosis of organs and vasculature leads to increased mortality and severely worsened quality of life. A potential unifying hypothesis to account for longstanding illness, examined in more detail below, is overexpression of TGF-β, which leads to a protracted state of immunosuppression and fibrosis. Histologic changes from the lungs of COVID-19 patients demonstrate fibroblastic proliferations and interstitial fibrosis, suggestive of TGF-β involvement (Oronsky, B., et al. 2021. Clinic Rev Allerg Immunol 20, 1-9). An inhaled formulation of FBM5712 and its potential to be delivered as an inhalable drug represents another opportunity in this emerging disease.

This evidence supports a central role for TGF-β in the pathogenesis of fibrosis, including human and murine pulmonary fibrosis. Approaches which disrupt the TβRI/Activin receptor-like kinase 5 (ALK5) receptor (Journal of Clinical Investigation (2011), 121(1), 277-287) have offered protection in experimental models of pulmonary fibrosis. Therapeutic treatment of bleomycin-challenged rats, after the onset of fibrosis, with the ALK5 inhibitor compound SB-525334, resulted in significant dose-dependent improvements in all parameters of lung function measured, as compared to vehicle-treated controls. These improvements in lung function correlated with a significant reduction in pulmonary pathology (Jarman et al Physiological Reports, (2014), 2(9), e12133). In another study, ALK5 inhibitor compound R-268712 had strong antifibrotic efficacy in the bleomycin model based on results from both the short-term luciferase and longer-term conventional assays (Terashima et al Pulmonary Pharmacology & Therapeutics (2019), 51, 31-38). Despite much effort made to date by a number of groups, clinical development of a therapeutic antagonist of the TGF-β pathway has been challenging as discussed.

Efforts reported thus far to inhibit the TGF-β pathway have focused on achieving systemic exposure of a therapeutic by targeting either the TGF-β ligands, or the kinase activity of the TGFβ receptor, ALK5. Observations from preclinical studies, including in rats and dogs, have revealed certain systemic toxicities associated with inhibition of TGF-β in vivo. Moreover, although several TGFβ/ALK5 inhibitors entered clinical trials, multiple clinical programs targeting TGF-β have been discontinued due to systemic side effects.

For example, Anderton et al. (Toxicologic Pathology (2011). 39(6), 916-924) reported that two small molecule inhibitors of ALK5, namely AZ12601011 and AZ12799734, induced heart valve lesions characterized by hemorrhage, inflammation, degeneration and proliferation of valvular interstitial cells in a preclinical animal model. The toxicity was observed in all heart valves at all doses tested. Frazier et al. (Toxicologic Pathology (2007). 35(2), 284-295) reported that administration of the ALK5 inhibitor GW788388 induced physeal dysplasia in rats.

Stauber et al. (Journal of Clinical Toxicology (2014), 4(3), 1000196/1-1000196/10) reported that a chronic (>3 months) administration of the ALK5 inhibitor LY2157299, which is being investigated for certain cancer treatments, caused multiple organ toxicities involving the cardiovascular, gastrointestinal, immune, bone/cartilage, reproductive, and renal systems, in rats and dogs. In should be noted however, such cardiovascular safety concerns may not occur in man (Kovacs et al 2015 Cardiovasc Toxicol. 2015: 15(4): 309-323).

Fresolimumab (GC1008), a “pan” TGF-β antibody capable of neutralizing all human isoforms of TGF-β, has been reported to induce an epithelial hyperplasia of the gingiva, bladder, and of the nasal turbinate epithelium after multiple administrations in studies with cynomolgus macaques (Current Pharmaceutical Biotechnology (2011), 12(12), 2176-2189). Similarly, a variety of bleeding and anemia issues prevented clinical development in scleroderma despite clear clinical anti-fibrotic efficacy (Rice et al 2015. J Clin Invest.; 125(7):2795-2807).

For ALK5 inhibitors in clinical development, a commonly used approach to manage toxicity concerns is to employ an intermittent dosing regimen, such as 14 days on, 14 days off schedule (Yap et al, Proceedings from the 2018 SITC Annual Meeting, Abstract 030). Continuous dosing of ALK5 inhibitors to achieve uninterrupted suppression of the pathway may have greater therapeutic benefit, but safety concerns have thus far prevented the use of continuous schedules with orally-dosed ALK5 inhibitors in the clinic.

Therefore, there is a need to deliver an ALK5 inhibitor directly through inhalation to the lungs and an advantage to deliver a soft ALK5 inhibitor to further minimize the potential for systemic toxicity and thereby achieve even greater therapeutic benefit.

Some embodiments provided herein relate to a method for treating a disease by inhalation of an ALK-5 inhibitor including, all types of pulmonary fibrosis. Pulmonary diseases such as interstitial lung disease including Idiopathic Pulmonary Fibrosis (IPF), Idiopathic Interstitial Pneumonia (IIP), scleroderma-associated interstitial lung disease (SSc-ILD), sarcoidosis, bronchiolitis obliterans, Langerhans cell histiocytosis (also called Eosinophilic granuloma or Histiocytosis X), chronic eosinophilic pneumonia, collagen vascular disease, granulomatous vasculitis, Goodpasture's syndrome, pulmonary alveolar proteinosis (PAP), and cystic fibrosis. Inhalation of an ALK-5 inhibitor could also be useful in the treatment of lung cancer.

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

As used herein, “alkyl” means a branched, or straight chain chemical group containing only carbon and hydrogen, such as methyl, ethyl, n-propyl, iso-propyl, n-butyl, iso-butyl, sec-butyl, tert-butyl, n-pentyl, iso-pentyl, sec-pentyl and neo-pentyl. Alkyl groups can either be unsubstituted or substituted with one or more substituents. In some embodiments, alkyl groups include 1 to 9 carbon atoms (for example, 1 to 6 carbon atoms, 1 to 4 carbon atoms, or 1 to 2 carbon atoms).

As used herein, “cycloalkyl” means a cyclic ring system containing only carbon atoms in the ring system backbone, such as cyclopropyl, cyclobutyl, cyclopentyl, cyclohexyl, and cyclohexenyl. Cycloalkyls may include multiple fused rings. Carbocyclyls may have any degree of saturation provided that none of the rings in the ring system are aromatic. Carbocyclyl groups can either be unsubstituted or substituted with one or more substituents. In some embodiments, carbocyclyl groups include 3 to 10 carbon atoms, for example, 3 to 6 carbon atoms.

As used herein, “aryl” means a mono-, bi-, tri- or polycyclic group with only carbon atoms present in the ring backbone having 5 to 14 ring atoms, alternatively 5, 6, 9, or 10 ring atoms; and having 6, 10, or 14 pi electrons shared in a cyclic array: wherein at least one ring in the system is aromatic. Aryl groups can either be unsubstituted or substituted with one or more substituents. Examples of aryl include phenyl, naphthyl, tetrahydronaphthyl, 2,3-dihydro-1H-indenyl, and others. In some embodiments, the aryl is phenyl.

As used herein, the term “heteroaryl” means a mono-, bi-, tri- or polycyclic group having 5 to 14 ring atoms, alternatively 5, 6, 9, or 10 ring atoms; and having 6, 10, or 14 pi electrons shared in a cyclic array: wherein at least one ring in the system is aromatic, and at least one ring in the system contains one or more heteroatoms independently selected from the group consisting of N, O, and S. Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[3,2-c]pyridinyl, thieno[3,2-b]pyridinyl, thieno[2,3-c]pyridinyl, thieno[2,3-b]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others. In some embodiments, the heteroaryl is selected from thienyl, pyridinyl, furyl, pyrazolyl, imidazolyl, isoindolinyl, pyranyl, pyrazinyl, and pyrimidinyl.

As used herein, “halo”, “halide” or “halogen” is a chloro, bromo, fluoro, or iodo atom radical. In some embodiments, a halo is a chloro, bromo or fluoro. For example, a halide can be fluoro.

As used herein, “haloalkyl” means a hydrocarbon substituent, which is a linear or branched, alkyl, alkenyl or alkynyl substituted with one or more chloro, bromo, fluoro, and/or iodo atom(s). In some embodiments, a haloalkyl is a fluoroalkyls, wherein one or more of the hydrogen atoms have been substituted by fluoro. In some embodiments, haloalkyls are of 1 to about 3 carbons in length (e.g., 1 to about 2 carbons in length or 1 carbon in length). The term “haloalkylene” means a diradical variant of haloalkyl, and such diradicals may act as spacers between radicals, other atoms, or between a ring and another functional group.

As used herein, “heterocycloalkyl” means a nonaromatic cyclic ring system comprising at least one heteroatom in the ring system backbone. Heterocyclyls may include multiple fused rings. Heterocyclyls may be substituted or unsubstituted with one or more substituents. In some embodiments, heterocycles have 3-11 members. In six membered monocyclic heterocycles, the heteroatom(s) are selected from one to three of O, N or S, and wherein when the heterocycle is five membered, it can have one or two heteroatoms selected from O, N, or S. Examples of heterocyclyl include azirinyl, aziridinyl, azetidinyl, oxetanyl, thietanyl, 1,4,2-dithiazolyl, dihydropyridinyl, 1,3-dioxanyl, 1,4-dioxanyl, 1,3-dioxolanyl, morpholinyl, thiomorpholinyl, piperazinyl, pyranyl, pyrrolidinyl, tetrahydrofuryl, tetrahydropyridinyl, oxazinyl, thiazinyl, thiinyl, thiazolidinyl, isothiazolidinyl, oxazolidinyl, isoxazolidinyl, piperidinyl, pyrazolidinyl imidazolidinyl, thiomorpholinyl, and others. In some embodiments, the heterocyclyl is selected from azetidinyl, morpholinyl, piperazinyl, pyrrolidinyl, and tetrahydropyridinyl.

The term “substituted” refers to moieties having substituents replacing a hydrogen on one or more non-hydrogen atoms of the molecule. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. Substituents can include, for example, —(C₁₋₉ alkyl) optionally substituted with one or more of hydroxyl, —NH₂, —NH(C₁₋₃ alkyl), and —N(C₁₋₃ alkyl)₂; —(C₁₋₉ haloalkyl); a halide; a hydroxyl; a carbonyl [such as —C(═O)OR, and —C(═O)R]; a thiocarbonyl [such as —C(═S)OR, —C(═O)SR, and —C(═S)R]; —(C₁₋₉ alkoxy) optionally substituted with one or more of halide, hydroxyl, —NH₂, —NH(C₁₋₃ alkyl), and —N(C₁₋₃ alkyl)₂; —OPO(OH)₂; a phosphonate [such as —PO(OH)₂ and —PO(OR′)₂]; —OPO(OR)R″; —NRR″; —C(O)NRR′; —C(NR)NR′R″; —C(NR′)R″; a cyano; a nitro; an azido; —SH; —S—R; —OSO₂(OR); a sulfonate [such as —SO₂(OH) and —SO₂(OR)]; —SO₂NR′R″; and —SO₂R; in which each occurrence of R, R′ and R″ are independently selected from H; —(C₁₋₉ alkyl); C₆₋₁₀ aryl optionally substituted with from 1-3R′″; 5-10 membered heteroaryl having from 1-4 heteroatoms independently selected from N, O, and S and optionally substituted with from 1-3 R′″; C₃-7 carbocyclyl optionally substituted with from 1-3 R′″; and 3-8 membered heterocyclyl having from 1-4 heteroatoms independently selected from N, O, and S and optionally substituted with from 1-3 R′″; wherein each R′″ is independently selected from —(C₁₋₆ alkyl), —(C₁₋₆ haloalkyl), a halide (e.g., F), a hydroxyl, —C(O)OR, —C(O)R, —(C₁₋₆ alkoxyl), —NRR′, —C(O)NRR′, and a cyano, in which each occurrence of R and R′ is independently selected from H and —(C₁₋₆ alkyl). In some embodiments, the substituent is selected from —(C₁₋₆ alkyl), —(C₁₋₆ haloalkyl), a halide (e.g., F), a hydroxyl, —C(O)OR, —C(O)R, —(C₁₋₆ alkoxyl), —NRR′, —C(O)NRR″, and a cyano, in which each occurrence of R and R′ is independently selected from H and —(C₁₋₆ alkyl).

As used herein, when two groups are indicated to be “linked” or “bonded” to form a “ring”, it is to be understood that a bond is formed between the two groups and may involve replacement of a hydrogen atom on one or both groups with the bond, thereby forming a carbocyclyl, heterocyclyl, aryl, or heteroaryl ring. The skilled artisan will recognize that such rings can and are readily formed by routine chemical reactions. In some embodiments, such rings have from 3-7 members, for example, 5 or 6 members.

The skilled artisan will recognize that some chemical structures described herein may be represented on paper by one or more other resonance forms: or may exist in one or more other tautomeric forms, even when kinetically, the artisan recognizes that such tautomeric forms represent only a very small portion of a sample of such compound(s). Such compounds are clearly contemplated within the scope of this disclosure, though such resonance forms or tautomers are not explicitly represented herein.

The compounds provided herein may encompass various stereochemical forms. The compounds also encompass diastereomers as well as optical isomers, e.g., mixtures of enantiomers including racemic mixtures, as well as individual enantiomers and diastereomers, which arise as a consequence of structural asymmetry in certain compounds. Separation of the individual isomers or selective synthesis of the individual isomers is accomplished by application of various methods which are well known to practitioners in the art. Unless otherwise indicated, when a disclosed compound is named or depicted by a structure without specifying the stereochemistry and has one or more chiral centers, it is understood to represent all possible stereoisomers of the compound.

The present disclosure includes all pharmaceutically acceptable isotopically labeled compounds of Formulas I, II, III, IV, V, VI, and VII wherein one or more atoms are replaced by atoms having the same atomic number, but an atomic mass or mass number different from the atomic mass or mass number which predominates in nature. Examples of isotopes suitable for inclusion in the compounds of the disclosure include, but are not limited to, isotopes of hydrogen, such as ²H (deuterium) and ³H (tritium), carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S.

The term “administration” or “administering” and “delivery” refers to a method of providing a dosage of a compound or pharmaceutical composition to a mammal, where the method is, e.g., orally, intranasally, intrapulmonarily, intraperitoneally, intrapleurally, intrabronchially, via inhalation, via endotracheal or endobronchial instillation, via direct instillation into pulmonary cavities, intrathoracically, and via thoracostomy irrigation. The method of administration can vary depending on various factors, e.g., the components of the pharmaceutical composition, the desired site at which the formulation is to be introduced, delivered or administered, the site where therapeutic benefit is sought, or the proximity of the initial delivery site to the downstream diseased organ e.g., aerosol delivery to the lung In some embodiments, pharmaceutical compositions described herein are administered by pulmonary administration.

The terms “pulmonary administration” or “inhalation” or “pulmonary delivery” or “oral inhalation” or “intranasal inhalation” and other related terms refers to a method of providing a dosage of a compound or pharmaceutical composition to a mammal, by a route such that the desired therapeutic or prophylactic agent is delivered to the lungs of the mammal. Such delivery to the lung may occur by intranasal administration, oral inhalation administration. Each of these routes of administration may occur as inhalation of an aerosol of formulations described herein. In some embodiments, pulmonary administration occurs by passively delivering an aerosol described herein by mechanical ventilation.

The terms “intranasal inhalation administration” and “intranasal inhalation delivery” refers to a method of providing a dosage of a compound or pharmaceutical composition to a mammal, by a route such that the formulation is targeting delivery and absorption of the therapeutic formulation directly in the lungs of the mammal through the nasal cavity. In some embodiments, intranasal inhalation administration is performed with a nebulizer.

The terms “intranasal administration” and “intranasal delivery” refer to a method of providing a dosage of a compound or pharmaceutical composition to a mammal, by a route such that the desired therapeutic or prophylactic agent is delivered via the nasal cavity. Such delivery to the nasal cavity may occur by intranasal administration, wherein this route of administration may occur as inhalation of an aerosol of formulations described herein, injection of an aerosol of formulations described herein, gavage of a formulation described herein, or passively delivered by mechanical ventilation.

The terms “oral inhalation administration” or “oral inhalation delivery” or “oral inhalation” refer to a method of providing a dosage of a compound or pharmaceutical composition to a mammal, through the mouth for delivery and absorption of the formulation directly to the lungs of the mammal. In some embodiments, oral inhalation administration is carried out by the use of a nebulizer.

The term “mammal” is used in its usual biological sense. Thus, it specifically includes humans, cattle, horses, monkeys, dogs, cats, mice, rats, cows, sheep, pigs, goats, and non-human primates, but also includes many other species.

The term “pharmaceutically acceptable carrier”, “pharmaceutically acceptable diluent” or “pharmaceutically acceptable excipient” includes any and all solvents, co-solvents, complexing agents, dispersion media, coatings, isotonic and absorption delaying agents and the like which are not biologically or otherwise undesirable. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. In addition, various adjuvants such as are commonly used in the art may be included. These and other such compounds are described in the literature, e.g., in the Merck Index, Merck & Company. Rahway, NJ. Considerations for the inclusion of various components in pharmaceutical compositions are described, e.g., in Brunton et al. (Eds.) (2017); Goodman and Gilman's: The Pharmacological Basis of Therapeutics, 13th Ed., The McGraw-Hill Companies.

The term “pharmaceutically acceptable salt” refers to salts that retain the biological effectiveness and properties of the compounds provided herein and, which are not biologically or otherwise undesirable. In many cases, the compounds provided herein are capable of forming acid and/or base salts by virtue of the presence of amino and/or carboxyl groups or groups similar thereto. Many such salts are known in the art, for example, as described in WO 87/05297. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and beryllium and the like: particularly preferred are the ammonium, potassium, sodium, calcium, and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, histidine, arginine, lysine, benethamine, N-methyl-glucamine, and ethanolamine. Other acids include dodecylsulfuric acid, naphthalene-1,5-disulfonic acid, naphthalene-2-sulfonic acid, and saccharin.

The term “pH-reducing acid” refers to acids that retain the biological effectiveness and properties of the compounds of this disclosure and, which are not biologically or otherwise undesirable. Pharmaceutically acceptable pH-reducing acids include, for example, inorganic acids such as, e.g., hydrochloric acid, hydrobromic acid, sulfuric acid, nitric acid, phosphoric acid, and the like. Also by nonlimiting example, pH-reducing acids may also include organic acids such as citric acid, acetic acid, propionic acid, naphtoic acid, oleic acid, palmitic acid, pamoic (emboic) acid, stearic acid, glycolic acid, pyruvic acid, oxalic acid, maleic acid, malonic acid, succinic acid, fumaric acid, tartaric acid, citric acid, ascorbic acid, glucoheptonic acid, glucuronic acid, lactic acid, lactobioic acid, tartaric acid, benzoic acid, cinnamic acid, mandelic acid, methanesulfonic acid, ethanesulfonic acid, p-toluenesulfonic acid, salicylic acid, and the like.

The term “acidic excipient” that is typically present as an acidic excipient aqueous solution. Examples of may include acid salts such as phosphate, sulphate, nitrate, acetate, formate, citrate, tartrate, propionate and sorbate, organic acids such as carboxylic acids, sulfonic acids, phosphonic acids, phosphinic acids, phosphoric monoesters, and phosphoric diesters, and/or other organic acids that contain from 1 to 12 carbon atoms, citric acid, acetic acid, formic acid, propionic acid, butyric acid, benzoic acid, mono-, di-, and trichloroacetic acid, salicylic acid, trifluoroacetic acid, benzenesulfonic acid, toluenesulfonic acid, methylphosphonic acid, methylphosphinic acid, dimethylphosphinic acid, and phosphonic acid monobutyl ester. It may include also stable or biodegradable poly-acidic polymers or co-polymers such as polycarbophil, acid celluloses, polyglycolides, polylactides and polymethacrylates.

“Patient” as used herein, means a human or a non-human mammal, e.g., a dog, a cat, a mouse, a rat, a cow, a sheep, a pig, a goat, a non-human primate, or a bird, e.g., a chicken, as well as any other vertebrate or invertebrate. In some embodiments, the patient is a human.

A “therapeutically effective amount” of a compound as provided herein is one which is sufficient to achieve the desired physiological effect and may vary according to the nature and severity of the disease condition, and the potency of the compound. “Therapeutically effective amount” is also intended to include one or more of the compounds of Formulas I, II, III, IV, V, VI, and VII in combination with one or more other agents that are effective to treat the diseases and/or conditions described herein. The combination of compounds can be a synergistic combination. Synergy, as described, for example, by Chou and Talalay, Advances in Enzyme Regulation (1984), 22, 27-55, occurs when the effect of the compounds when administered in combination is greater than the additive effect of the compounds when administered alone as a single agent. In general, a synergistic effect is most clearly demonstrated at sub-optimal concentrations of the compounds. It will be appreciated that different concentrations may be employed for prophylaxis than for treatment of an active disease. This amount can further depend upon the patient's height, weight, sex, age and medical history.

A therapeutic effect relieves, to some extent, one or more of the symptoms of the disease.

“Treat,” “treatment,” or “treating,” as used herein refers to administering a compound or pharmaceutical composition as provided herein for therapeutic purposes. The term “therapeutic treatment” refers to administering treatment to a patient already suffering from a disease thus causing a therapeutically beneficial effect, such as ameliorating existing symptoms, ameliorating the underlying metabolic causes of symptoms, postponing or preventing the further development of a disorder, and/or reducing the severity of symptoms that will or are expected to develop.

The term “dosing interval” refers to the time between administrations of the two sequential doses of a pharmaceutical's during multiple dosing regimens.

The term “respirable delivered dose” refers to the amount of aerosolized compound particles inhaled during the inspiratory phase of the breath simulator that is equal to or less than 5 microns.

The term “lung deposition” as used herein, refers to the fraction of the nominal dose of an active pharmaceutical ingredient (API) that is deposited on the inner surface of the lungs.

The term “nominal dose,” or “loaded dose” refers to the amount of drug that is placed in the nebulizer prior to administration to a mammal. The volume of solution containing the nominal dose is referred to as the “fill volume.”

The term “enhanced pharmacokinetic profile” refers to an improvement in some pharmacokinetic parameter. Pharmacokinetic parameters that may be improved include, AUCₗₐₛₜ, AUC_{(0-∞)}, Tₘₐₓ, and optionally a Cₘₐₓ. In some embodiments, the enhanced pharmacokinetic profile may be measured quantitatively by comparing a pharmacokinetic parameter obtained for a nominal dose of an active pharmaceutical ingredient (API) administered with one type of inhalation device with the same pharmacokinetic parameter obtained with oral administration of a composition of the same active pharmaceutical ingredient (API).

The term “blood plasma concentration” refers to the concentration of an active pharmaceutical ingredient (API) in the plasma component of blood of a subject or patient population.

The term “respiratory condition,” as used herein, refers to a disease or condition that is physically manifested in the respiratory tract, including, but not limited to, pulmonary fibrosis, chronic obstructive pulmonary disease (COPD), bronchitis, chronic bronchitis, emphysema, or asthma.

The term “metered-dose inhaler” as used herein, refers to a device that delivers a specific amount of medication to the lungs, in the form of a short burst of aerosolized medicine that is usually self-administered by the patient via inhalation.

The term “dry powder inhalers” as used herein, refers to a device that delivers medication to the lungs in the form of a dry powder. There are several designs of dry powder inhalers. For example, one design is the metering device in which a reservoir for the drug is placed within the device and the patient adds a dose of the drug into the inhalation chamber. Another method is a factory-metered device in which each individual dose has been manufactured in a separate container. Both systems depend upon the formulation of drug into small particles of mass median diameters from about 1 to about 5 microns, and usually involve co-formulation with larger excipient particles (typically 100-micron diameter lactose particles). Drug powder is placed into the inhalation chamber (either by device metering or by breakage of a factory-metered dosage) and the inspiratory flow of the patient accelerates the powder out of the device and into the oral cavity.

The term “nebulizer,” as used herein, refers to a device that turns medications, compositions, formulations, suspensions, and mixtures, etc. into a fine mist or aerosol for delivery to the lungs. Nebulizers may also be referred to as atomizers.

The term “soft mist inhaler” as used herein, refers to a device that provides a metered dose to the user, as the liquid bottom of the inhaler is rotated clockwise 180 degrees by hand, adding a buildup tension into a spring around the flexible liquid container. When the user activates the bottom of the inhaler, the energy from the spring is released and imposes pressure on the flexible liquid container, causing liquid to spray out of two nozzles, thus forming a soft mist to be inhaled. An example is the Respimat® Soft Mist™ Inhaler.

The term “jet nebulizer” as used herein, refers to a device that utilizes air pressure breakage of an aqueous solution into aerosol droplets. Jet nebulizers are connected by tubing to a supply of compressed gas, usually compressed air or oxygen to flow at high velocity through a liquid medicine to turn it into an aerosol, which is then inhaled by the patient.

The term “ultrasonic nebulizer” as used herein, refers to a device that utilizes an electronic oscillator to generate high frequency ultrasonic waves, which causes the mechanical vibration of a piezoelectric element. This vibrating element is in contact with a liquid reservoir and its high frequency vibration is sufficient to produce a vapor mist at the liquid surface. Non-limiting examples are the Omron NE-U17 and Beurer Nebulizer IH30.

The term “vibrating mesh nebulizer” as used herein, refers to a device that are driven by a piezo-element and use ultrasonic frequencies to vibrate a mesh/membrane with 1000-7000 laser drilled holes vibrates at the top of the liquid reservoir, and thereby pressures out a mist of very fine droplets through the holes. Non-limiting examples are Pari eFlow® rapid Nebulizer System, Respironics I-neb, Beurer Nebulizer IH50, and Aerogen Aeroneb.

The term “breath-actuated nebulizer” as used herein, refers to a device that produces aerosol during inspiration, when the negative pressure generated by the patient is sufficient to pull the actuator down into position, sealing the jet nozzle to allow medication to be drawn from the reservoir, generating aerosol. An example is the AeroEclipse® Breath Actuated Nebulizer.

The term “high efficiency liquid nebulizers” as used herein, refers to a device that delivers a large fraction of a loaded dose to a patient. Some high efficiency liquid nebulizers utilize one or more of the following: microperforated membranes, actively or passively vibrating microperforated membranes, oscillating membranes, a vibrating mesh or plate with multiple apertures, a vibration generator with an aerosol mixing chamber, a resonant system, and/or a pulsating membrane. Some high efficiency liquid nebulizers are continuously operating. Some contain a vibrating microperforated membrane of tapered nozzles against a bulk liquid to generate a plume of droplets without the need for compressed gas. Some use passive nozzle membranes and a separate piezoelectric transducer that are in contact with the solution. Some employ an active nozzle membrane, which use the acoustic pressure in the nebulizer to generate very fine droplets of solution via the high frequency vibration of the nozzle membrane.

The term “about” when used to refer to a quantitative value means that a specified quantity may be greater than or less than the indicated amount by 1, 2, 3, 4, 5, 6, 7, 8, 9, 10, 11, 12, 13, 14, 15, 16, 17, 18, 19 or 20 percent of the stated numerical value.

### Compounds

The compounds and compositions described herein can be used as anti-fibrotic agents. In addition, the compounds can be used as inhibitors of one or more activin receptor-like kinases (ALKs). ALKs are part of the TGF-β superfamily of receptors which has been implicated into different physiological and pathological processes in a broad range of cell systems, including fibroblasts, immune, stem, endothelial, mural and tumor cells.

The compounds and compositions described herein may also be used to alleviate any type of TGF-β-mediated condition. Examples of the TGF-β-mediated conditions include lung cancer, as well as all types of fibrotic pulmonary diseases. In one embodiment, the TGF-β-mediated condition is idiopathic pulmonary fibrosis. In another embodiment it is lung fibrosis that occurs in patients with scleroderma, also known as systemic sclerosis.

In some embodiments, compounds for use as ALK5 inhibitors include the compounds set forth below as described in the following journal articles, U.S. patents and U.S. patent applications.

In some embodiments, an ALK5 inhibitor compound is a compound described in any of US patent publication no. 20080090861; U.S. Pat. Nos. 7,964,612; 8,455,512; 9,090,625; or U.S. Pat. No. 9,260,450, the contents of each of which are hereby incorporated herein.

Some embodiments of the present disclosure include compounds of Formula I:

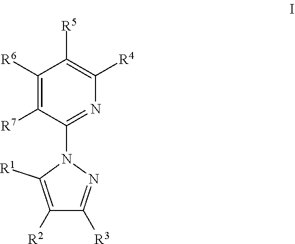

or salts, pharmaceutically acceptable salts, or prodrugs thereof.

In some embodiments, R¹ is selected from the group consisting of thieno[3,2-c]pyridinyl, thieno[3,2-b]pyridinyl, thieno[2,3-c]pyridinyl, and thieno[2,3-b]pyridinyl: wherein each may be optionally substituted with one to three substituents each independently selected from the group consisting of C₁-C₃-alkyl, —(C₁-C₃-alkyl)S(C₁-C₃-alkyl), —S(C₁-C₃-alkyl), —(C₁-C₃-alkyl)O(C₁-C₃-alkyl), —O(C₁-C₃-alkyl), —C(═O)O(C₁-C₃-alkyl), —CO₂H, —C(═O)NR⁸R⁹, halo, —CN, and —OH.

In some embodiments, R² and R³ are independently selected from the group consisting of H, C₁-C₃-alkyl, —(C₁-C₃-alkyl)S(C₁-C₃-alkyl), —S(C₁-C₃-alkyl), —(C₁-C₃-alkyl)O(C₁-C₃-alkyl), —O(C₁-C₃-alkyl), —C(═O)O(C₁-C₃-alkyl), —CO₂H, —C(═O)NR¹⁰R¹¹, halo, —CN, —OH, and C₃-C₆-cycloalkyl:

In some embodiments, R² and R³ may be taken together to form a 5-6-membered heteroaryl, phenyl, a C₄-C₆-cycloalkyl, or a 4-6-membered heterocycloalkyl; wherein C₄-C₆-cycloalkyl and 4-6-membered heterocycloalkyl may be optionally substituted with one to three substituents independently selected from the group consisting of halo, —OH, oxo, and C₁-C₃ alkyl; wherein 5-6-membered heteroaryl and phenyl may be optionally substituted with one to three substituents independently selected from the group consisting of halo, —CN, —OH, —O(C₁-C₃ alkyl), and C₁-C₃ alkyl.

In some embodiments, R⁴, R⁵, R⁶, and R⁷ are selected from the group consisting of H, C₃-cycloalkyl, C₁-C₃-alkyl, —(C₁-C₃-alkyl)S(C₁-C₃-alkyl), —S(C₁-C₃-alkyl), —(C₁-C₃-alkyl)O(C₁-C₃-alkyl), —O(C₁-C₃-alkyl), —C(═O)O(C₁-C₃-alkyl), —CO₂H, —C(═O)NR¹²R¹³ halo, —CN, —OH.

In some embodiments, R⁸ and R⁹ are each independently selected from the group consisting of H, and —(C₁-C₃ alkyl)OH, C₁-C₃-alkyl, halo, and —O(C₁-C₃-alkyl).

In some embodiments, R¹⁰ and R¹¹ are each independently selected from the group consisting of H and C₁-C₃ alkyl.

In some embodiments, R¹² and R¹³ are each independently selected from the group consisting of H, C₁-C₃ alkyl, halo, and —O(C₁-C₃-alkyl).

In some embodiments, R¹ is an unsubstituted thieno[3,2-c]pyridinyl.

In some embodiments, R¹ is an unsubstituted thieno[3,2-b]pyridinyl.

In some embodiments, R¹ is an unsubstituted thieno[2,3-c]pyridinyl.

In some embodiments, R¹ is an unsubstituted thieno[2,3-b]pyridinyl.

In some embodiments, R² and R³ are independently selected from the group consisting of H, C₁-C₃-alkyl, halo, —CN, and —OH.

In some embodiments, R² and R³ are independently selected from the group consisting of H, C₁-C₃-alkyl, and halo.

In some embodiments, R² and R³ are independently selected from the group consisting of H and C₁-C₃-alkyl:

In some embodiments, R² and R³ are both H; in some embodiments, R² and R³ are both C₁-C₃-alkyl; in some embodiments, R² and R³ are both Me; in some embodiments, R² and R³ are both Et; in some embodiments, R² is Me and R³ is Et; in some embodiments, R² is Et and R³ is Me; in some embodiments, R² is H and R³ is C₁-C₃-alkyl; in some embodiments, R² is C₁-C₃-alkyl and R³ is H; in some embodiments, R² is H and R³ is Me; in some embodiments, R² is Me and R³ is H; in some embodiments, R² is H and R³ is Et; and in some embodiments, R² is Et and R³ is H.

In some embodiments, R² and R³ are taken together to form a C₄-C₆-cycloalkyl; in some embodiments, R² and R³ are taken together to form a cyclobutyl; in some embodiments, R² and R³ are taken together to form a cyclopentyl; and in some embodiments, R² and R³ are taken together to form a cyclohexyl.

In some embodiments, R⁴, R⁵, R⁶, and R⁷ are selected from the group consisting of H, C₁-C₃-alkyl, halo, —CN, —OH.

In some embodiments, R⁴, R⁵, R⁶, and R⁷ are selected from the group consisting of H, C₁-C₃-alkyl, and halo.

In some embodiments, R⁴, R⁵, R⁶, and R⁷ are selected from the group consisting of H and C₁-C₃-alkyl.

In some embodiments, R⁴, R⁵, R⁶, and R⁷ are all H; in some embodiments, R⁴, R⁵, and R⁶ are all H and R⁷ is C₁-C₃-alkyl;

In some embodiments, R⁴, R⁵, and R⁷ are all H and R⁶ is C₁-C₃-alkyl: in some embodiments, R⁴, R⁶, and R⁷ are all H and R⁵ is C₁-C₃-alkyl; in some embodiments, R⁵, R⁶, and R⁷ are all H and R⁴ is C₁-C₃-alkyl; in some embodiments, R⁴, R⁵, and R⁶ are all H and R⁷ is Me; in some embodiments, R⁴, R⁵, and R⁷ are all H and R⁶ is Me; in some embodiments, R⁵, R⁶, and R⁷ are all H and R⁵ is Me; in some embodiments, R⁵, R⁶, and R⁷ are all H and R⁴ is Me; in some embodiments, R⁴ and R⁵ are both H and R⁶ and R⁷ are both C₁-C₃-alkyl; in some embodiments, R⁴ and R⁶ are both H and R⁵ and R⁷ are both C₁-C₃-alkyl; in some embodiments, R⁵ and R⁶ are both H and R⁴ and R⁷ are both C₁-C₃-alkyl; in some embodiments, R⁴ and R⁷ are both H and R⁵ and R⁶ are both C₁-C₃-alkyl; in some embodiments, R⁶ and R⁷ are both H and R⁴ and R⁵ are both C₁-C₃-alkyl; in some embodiments, R⁵ and R⁷ are both H and R⁴ and R⁶ are both C₁-C₃-alkyl: in some embodiments, R⁴ and R⁵ are both H and R⁶ and R⁷ are both Me; in some embodiments, R⁴ and R⁶ are both H and R⁵ and R⁷ are both Me; in some embodiments, R⁵ and R⁶ are both H and R⁴ and R⁷ are both Me; in some embodiments, R⁴ and R⁷ are both H and R⁵ and R⁶ are both Me: in some embodiments, R⁶ and R⁷ are both H and R⁴ and R⁵ are both Me; in some embodiments, R⁵ and R⁷ are both H and R⁴ and R⁶ are both Me; in some embodiments, R⁴, R⁵, and R⁶ are all C₁-C₃-alkyl and R⁷ is H; in some embodiments, R⁴, R⁵, and R⁷ are all C₁-C₃-alkyl and R⁶ is H; in some embodiments, R⁴, R⁶, and R⁷ are all C₁-C₃-alkyl and R$ is H; in some embodiments, R⁵, R⁶, and R⁷ are all C₁-C₃-alkyl and R⁴ is H; in some embodiments, R⁴, R⁵, and R⁶ are all Me and R⁷ is H: in some embodiments, R⁴, R⁵, and R⁷ are all Me and R⁶ is H; in some embodiments, R⁴, R⁶, and R⁷ are all Me and R⁵ is H; and in some embodiments, R⁵, R⁶, and R⁷ are all Me and R⁴ is H.

In some embodiments of Formula (I):
R¹ is selected from the group consisting of thieno[3,2-c]pyridinyl and thieno[2,3-c]pyridinyl optionally substituted with one to two substituents each independently selected from the group consisting of Me, halo, —CN, and —OH;
R² and R³ are independently selected from the group consisting of H, C₁-C₃-alkyl, halo, —CN, and —OH;
alternatively, R² and R³ may be taken together to form a C₄-C₆-cycloalkyl, optionally substituted with one to two substituents independently selected from the group consisting of halo, —OH, oxo, and Me; and
R⁴, R⁵, R⁶, and R⁷ are selected from the group consisting of H, C₁-C₃-alkyl, halo, —CN, —OH.

In certain embodiments, R¹ is a thieno[3,2-c]pyridinyl, which may be optionally substituted as specified herein. The positions of a thieno[3,2-c]pyridine are numbered as follows:

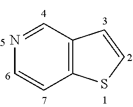

A thieno[3,2-c]pyridinyl is a monovalent radical of thieno[3,2-c]pyridine. Thus, in certain embodiments of the present invention, are compounds of Formula Ia:

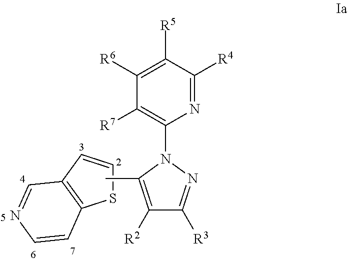

wherein R¹, R², R³, R⁴, R⁵, R⁶, and R⁷ have any of the values specified herein, and wherein the thieno[3,2-c]pyridinyl radical is attached at any of positions 2, 3, 4, 6, or 7.

In certain embodiments, R¹ is a thieno[2,3-c]pyridinyl, which may be optionally substituted as specified. The positions of a thieno[2,3-c]pyridine are numbered as follows:

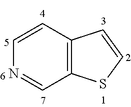

A thieno[2,3-c]pyridinyl is a monovalent radical of thieno[2,3-c]pyridine. Thus, in certain embodiments of the present invention, are compounds of Formula Ib:

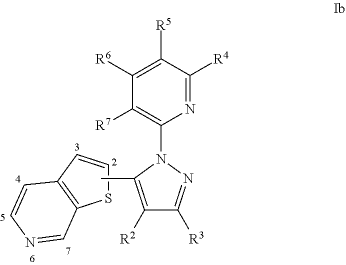

wherein R¹, R², R³, R⁴, R⁵, R⁶, and R⁷ have any of the values specified herein, and wherein the thieno[2,3-c]pyridinyl radical is attached at any of positions 2, 3, 4, 6, or 7.

In certain embodiments, R¹ is a thieno[2,3-b]pyridinyl, which may be optionally substituted as specified herein. The positions of a thieno[2,3-b]pyridine are numbered as follows:

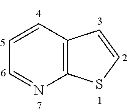

A thieno[2,3-b]pyridinyl is a monovalent radical of thieno[2,3-c]pyridine. Thus, in certain embodiments of the present invention, are compounds of Formula Ic:

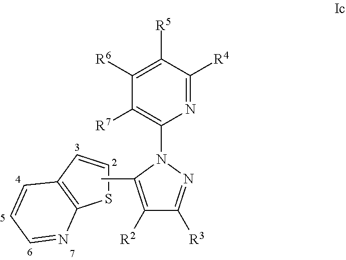

wherein R¹, R², R³, R⁴, R⁵, R⁶, and R⁷ have any of the values specified herein, and wherein the thieno[2,3-b]pyridinyl radical is attached at any of positions 2, 3, 4, 6, or 7.

In certain embodiments, R¹ is a thieno[3,2-b]pyridinyl, which may be optionally substituted as specified herein. The positions of a thieno[3,2-b]pyridine are numbered as follows:

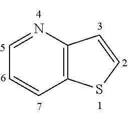

A thieno[3,2-b]pyridinyl is a monovalent radical of thieno[3,2-c]pyridine. Thus, in certain embodiments of the present invention, are compounds of Formula Id:

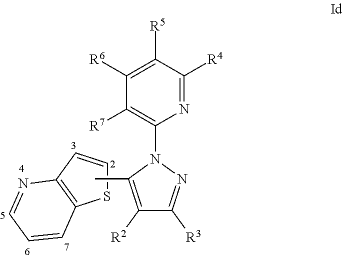

wherein R¹, R², R³, R⁴, R⁵, R⁶, and R⁷ have any of the values specified herein, and wherein the thieno[3,2-b]pyridinyl radical is attached at any of positions 2, 3, 4, 6, or 7.

In certain embodiments, the ALK5 (TGFβR¹) inhibitor is selected from one of the structures below or a pharmaceutically acceptable salt thereof,

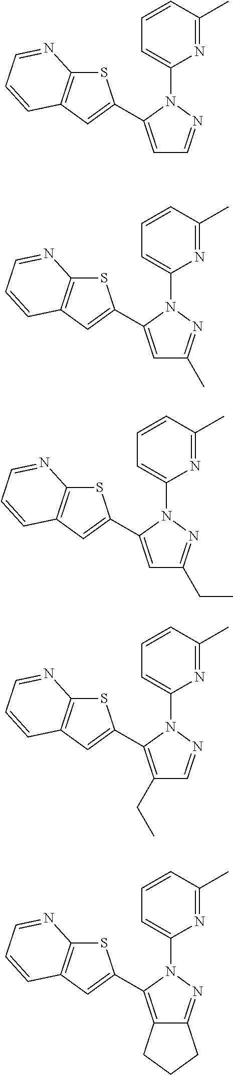

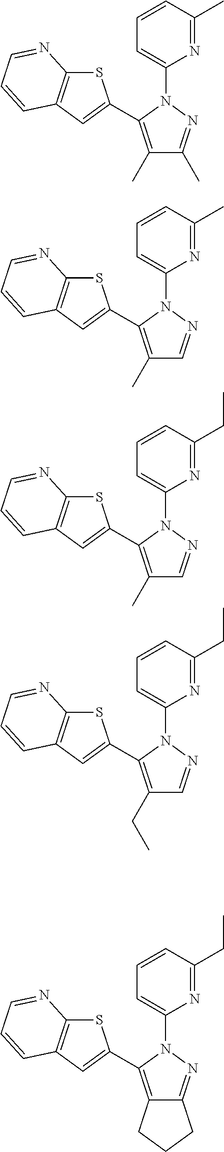

In certain embodiments, the ALK5 (TGFβR¹) inhibitor is selected from one of the structures below or a pharmaceutically acceptable salt thereof,

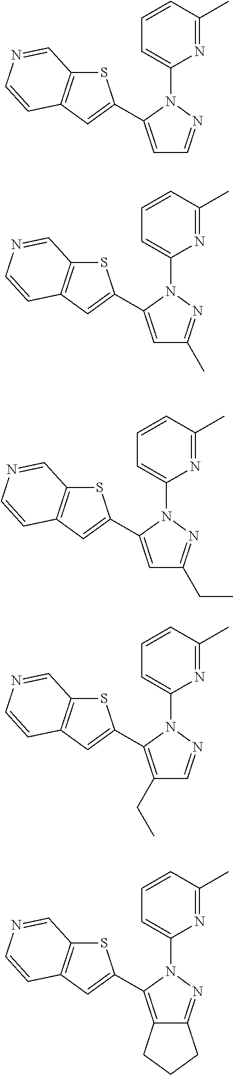

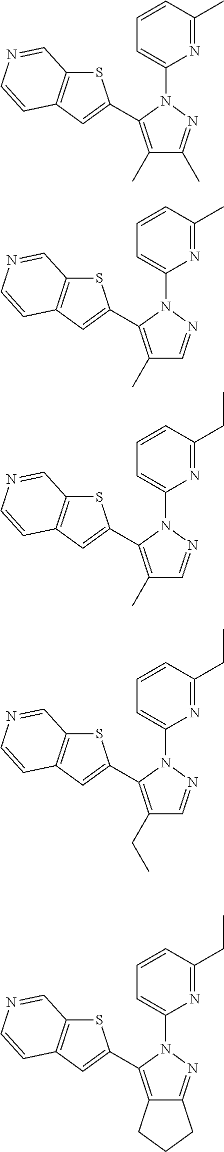

In certain embodiments, the ALK5 (TGFβR¹) inhibitor is selected from one of the structures below or a pharmaceutically acceptable salt thereof,

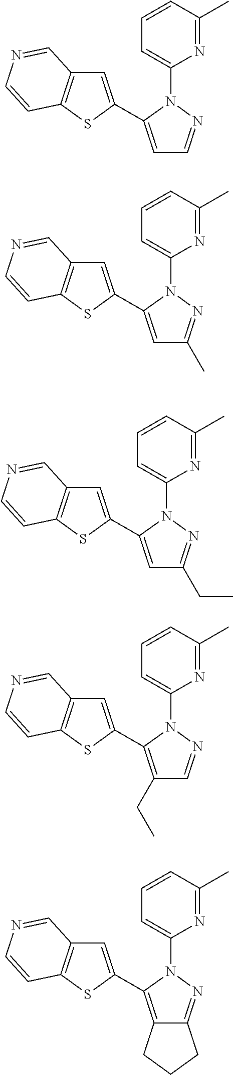

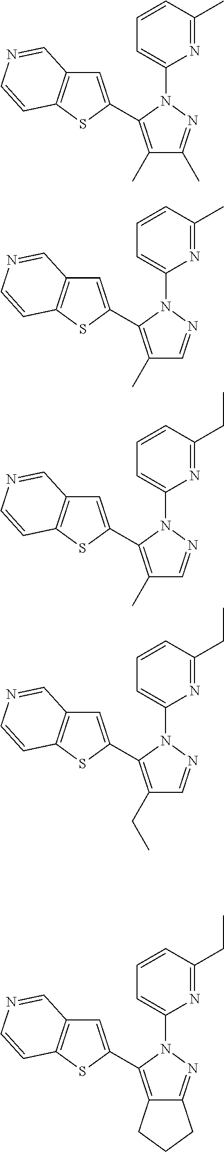

In certain embodiments, the ALK5 (TGFβR¹) inhibitor is selected from one of the structures below or a pharmaceutically acceptable salt thereof,

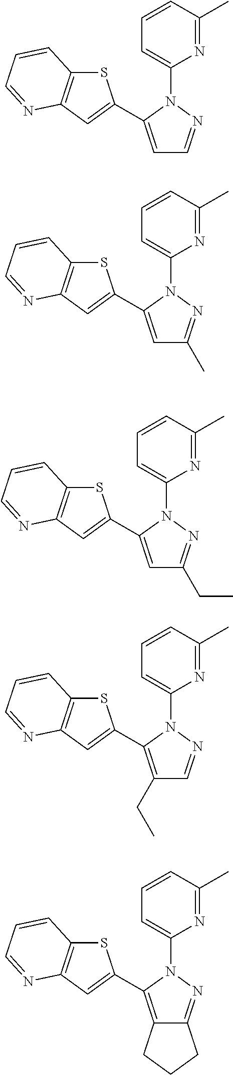

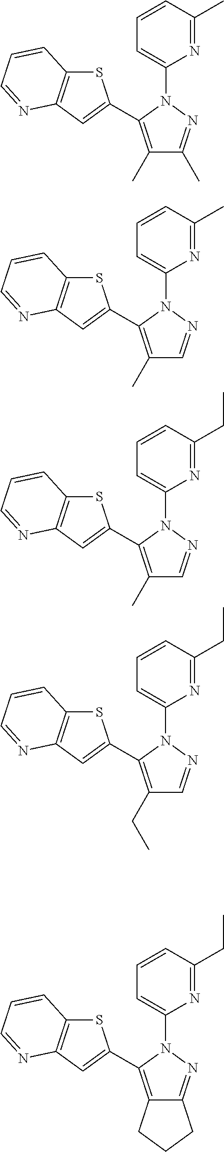

In certain preferred embodiments, the ALK5 (TGFβR¹) inhibitor is 2-(2-(6-methylpyridin-2-yl)-2,4,5,6-tetrahydrocyclopenta[c]pyrazol-3-yl)thieno[2,3-c]pyridine (shown below) or a pharmaceutically acceptable salt thereof,

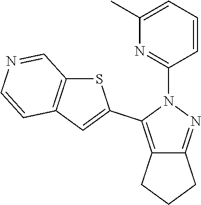 [SMILES: Cc1cccc(-n2nc3c(c2-c2cc4ccncc4s2)CCC3)n1]

In certain preferred embodiments, the ALK5 (TGFβR¹) inhibitor is 2-(4-methyl-1-(6-methylpyridin-2-yl)-1H-pyrazol-5-yl)thieno[2,3-c]pyridine (shown below) or a pharmaceutically acceptable salt thereof,

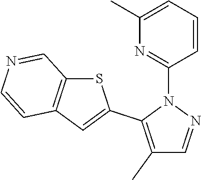 [SMILES: Cc1cccc(-n2ncc(C)c2-c2cc3ccncc3s2)n1]

In certain preferred embodiments, the ALK5 (TGFβR¹) inhibitor is 2-(2-(6-methylpyridin-2-yl)-2,4,5,6-tetrahydrocyclopenta[c]pyrazol-3-yl)thieno[3,2-c]pyridine (shown below) or a pharmaceutically acceptable salt thereof,

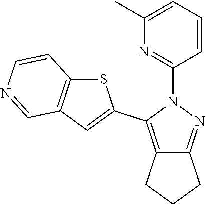 [SMILES: Cc1cccc(-n2nc3c(c2-c2cc4cnccc4s2)CCC3)n1]

In certain preferred embodiments, the ALlk5 (TGFβR¹) inhibitor is 2-[4-methyl-1-(6-methylpyridin-2-yl)-1H-pyrazol-5-yl]thieno[3,2-c]pyridine (shown below) or a pharmaceutically acceptable salt thereof,

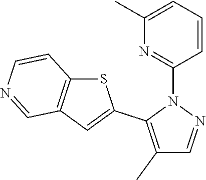 [SMILES: Cc1cccc(-n2ncc(C)c2-c2cc3cnccc3s2)n1]

Illustrative compounds of Formula (I) are shown in Table 1.

TABLE 1

| | |
|---|---|
| 1 | 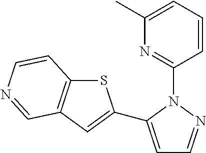 [SMILES: Cc1cccc(-n2nccc2-c2cc3cnccc3s2)n1] |
| 2 | 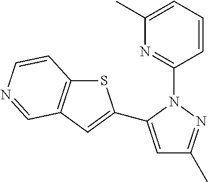 [SMILES: Cc1cccc(-n2nc(C)cc2-c2cc3cnccc3s2)n1] |
| 3 | 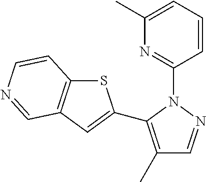 [SMILES: Cc1cccc(-n2ncc(C)c2-c2cc3cnccc3s2)n1] |
| 4 | 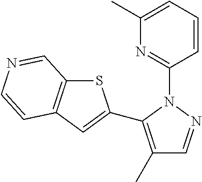 [SMILES: Cc1cccc(-n2ncc(C)c2-c2cc3ccncc3s2)n1] |
| 5 | 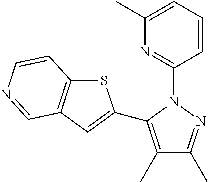 [SMILES: Cc1cccc(-n2nc(C)c(C)c2-c2cc3cnccc3s2)n1] |
| 6 | 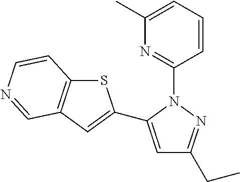 [SMILES: CCc1cc(-c2cc3cnccc3s2)n(-c2cccc(C)n2)n1] |
| 7 | 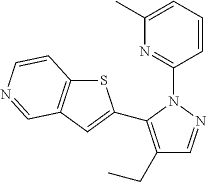 [SMILES: CCc1cnn(-c2cccc(C)n2)c1-c1cc2cnccc2s1] |
| 8 | 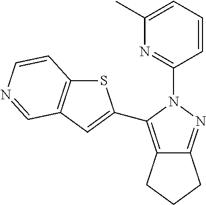 [SMILES: Cc1cccc(-n2nc3c(c2-c2cc4cnccc4s2)CCC3)n1] |
| 9 | 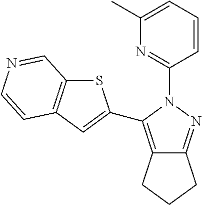 [SMILES: Cc1cccc(-n2nc3c(c2-c2cc4ccncc4s2)CCC3)n1] |
| 10 | 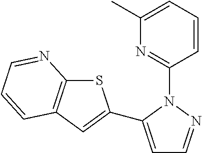 [SMILES: Cc1cccc(-n2nccc2-c2cc3cccnc3s2)n1] |
| 11 | 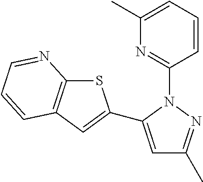 [SMILES: Cc1cccc(-n2nc(C)cc2-c2cc3cccnc3s2)n1] |
| 12 | 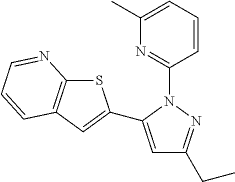 [SMILES: CCc1cc(-c2cc3cccnc3s2)n(-c2cccc(C)n2)n1] |
| 13 | 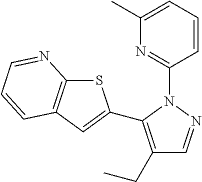 [SMILES: CCc1cnn(-c2cccc(C)n2)c1-c1cc2cccnc2s1] |
| 14 | 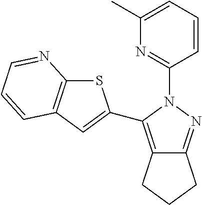 [SMILES: Cc1cccc(-n2nc3c(c2-c2cc4cccnc4s2)CCC3)n1] |
| 15 | 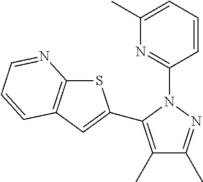 [SMILES: Cc1cccc(-n2nc(C)c(C)c2-c2cc3cccnc3s2)n1] |
| 16 | 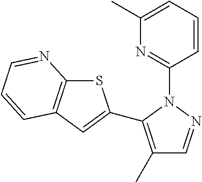 [SMILES: Cc1cccc(-n2ncc(C)c2-c2cc3cccnc3s2)n1] |
| 17 | 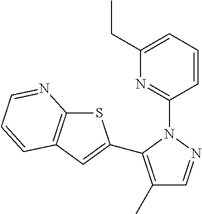 [SMILES: CCc1cccc(-n2ncc(C)c2-c2cc3cccnc3s2)n1] |
| 18 | 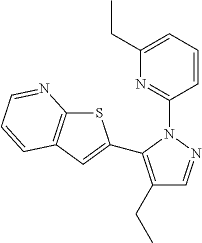 [SMILES: CCc1cccc(-n2ncc(CC)c2-c2cc3cccnc3s2)n1] |
| 19 | 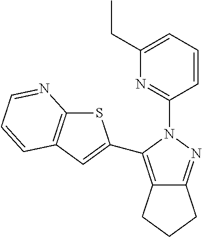 [SMILES: CCc1cccc(-n2nc3c(c2-c2cc4cccnc4s2)CCC3)n1] |
| 20 | 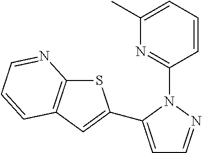 [SMILES: Cc1cccc(-n2nccc2-c2cc3cccnc3s2)n1] |
| 21 | 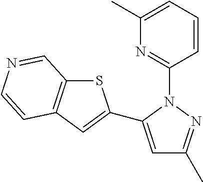 [SMILES: Cc1cccc(-n2nc(C)cc2-c2cc3ccncc3s2)n1] |
| 22 | 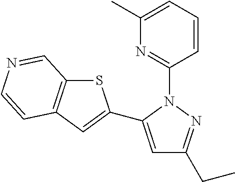 [SMILES: CCc1cc(-c2cc3ccncc3s2)n(-c2cccc(C)n2)n1] |
| 23 | 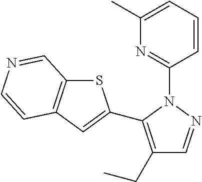 [SMILES: CCc1cnn(-c2cccc(C)n2)c1-c1cc2ccncc2s1] |
| 24 | 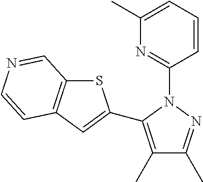 [SMILES: Cc1cccc(-n2nc(C)c(C)c2-c2cc3ccncc3s2)n1] |
| 25 | 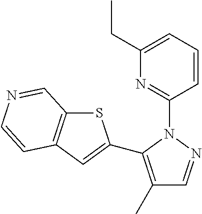 [SMILES: CCc1cccc(-n2ncc(C)c2-c2cc3ccncc3s2)n1] |
| 26 | 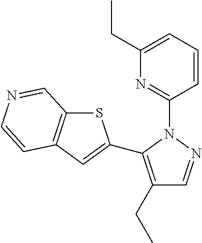 [SMILES: CCc1cccc(-n2ncc(CC)c2-c2cc3ccncc3s2)n1] |
| 27 | 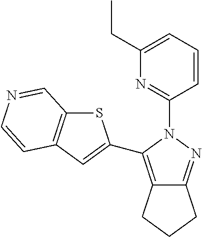 [SMILES: CCc1cccc(-n2nc3c(c2-c2cc4ccncc4s2)CCC3)n1] |
| 28 | 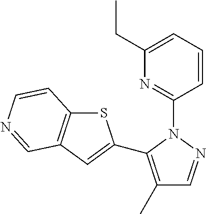 [SMILES: CCc1cccc(-n2ncc(C)c2-c2cc3cnccc3s2)n1] |
| 29 | 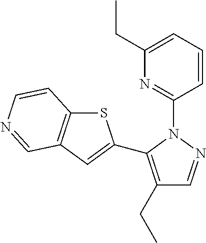 [SMILES: CCc1cccc(-n2ncc(CC)c2-c2cc3cnccc3s2)n1] |
| 30 | 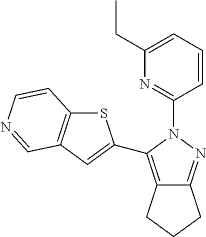 [SMILES: CCc1cccc(-n2nc3c(c2-c2cc4cnccc4s2)CCC3)n1] |
| 31 | 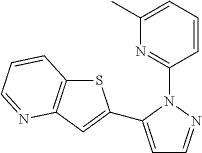 [SMILES: Cc1cccc(-n2nccc2-c2cc3ncccc3s2)n1] |
| 32 | 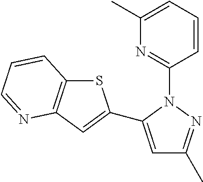 [SMILES: Cc1cccc(-n2nc(C)cc2-c2cc3ncccc3s2)n1] |
| 33 | 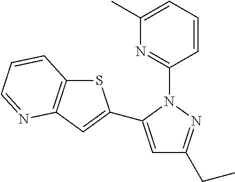 [SMILES: CCc1cc(-c2cc3ncccc3s2)n(-c2cccc(C)n2)n1] |
| 34 | 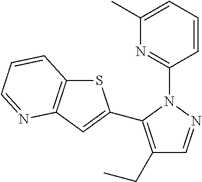 [SMILES: CCc1cnn(-c2cccc(C)n2)c1-c1cc2ncccc2s1] |
| 35 | 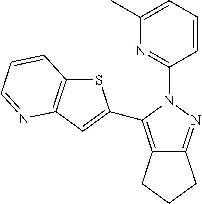 [SMILES: Cc1cccc(-n2nc3c(c2-c2cc4ncccc4s2)CCC3)n1] |
| 36 | 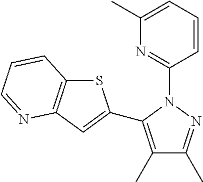 [SMILES: Cc1cccc(-n2nc(C)c(C)c2-c2cc3ncccc3s2)n1] |
| 37 | 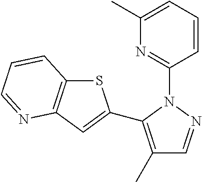 [SMILES: Cc1cccc(-n2ncc(C)c2-c2cc3ncccc3s2)n1] |
| 38 | 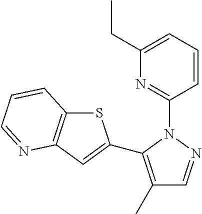 [SMILES: CCc1cccc(-n2ncc(C)c2-c2cc3ncccc3s2)n1] |
| 39 | 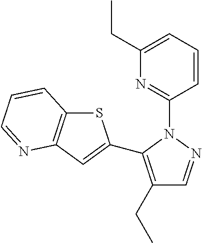 [SMILES: CCc1cccc(-n2ncc(CC)c2-c2cc3ncccc3s2)n1] |
| 40 | 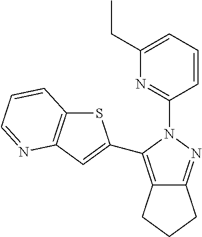 [SMILES: CCc1cccc(-n2nc3c(c2-c2cc4ncccc4s2)CCC3)n1] |

World Intellectual Property Organization. WO/2002/094833A1 and WO/2004/048382A1, describe compounds having Formula II and are hereby incorporated by reference in their entirety.

Some embodiments of the present disclosure include compounds of Formula II:

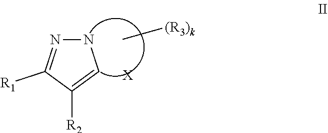

or salts, pharmaceutically acceptable salts, or prodrugs thereof.

An illustrative compound of Formula (II) is shown in Table 2.

TABLE 2

| | |
|---|---|
| 41 | 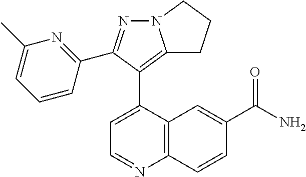 [SMILES: Cc1cccc(-c2nn3c(c2-c2ccnc4ccc(C(N)=O)cc24)CCC3)n1] |
| 42 | 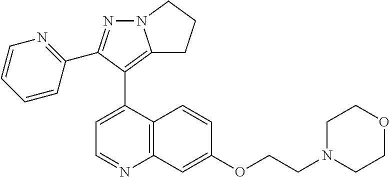 [SMILES: c1ccc(-c2nn3c(c2-c2ccnc4cc(OCCN5CCOCC5)ccc24)CCC3)nc1] |

In some embodiment, compound 41 is referred to as galunisertib (LY2157299) and compound 41 is referred to as LY2109761.

U.S. application No. 20110319406, describe compounds having Formula III and are hereby incorporated by reference in its entirety.

Some embodiments of the present disclosure include compounds of Formula III:

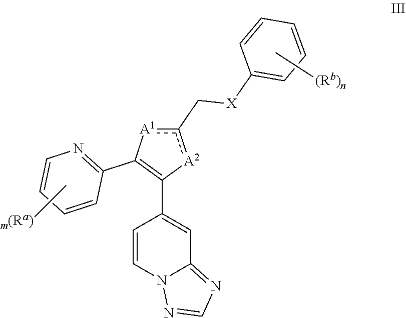

or salts, pharmaceutically acceptable salts, or prodrugs thereof.

An illustrative compound of Formula (III) is shown in Table 3.

TABLE 3

| | |
|---|---|
| 43 | 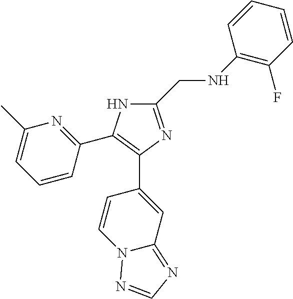 [SMILES: Cc1cccc(-c2[nH]c(CNc3ccccc3F)nc2-c2ccn3ncnc3c2)n1] |
| 44 | 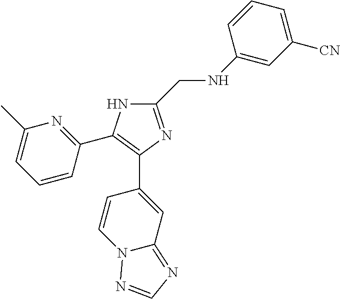 [SMILES: Cc1cccc(-c2[nH]c(CNc3cccc(C#N)c3)nc2-c2ccn3ncnc3c2)n1] |

In some embodiment, compound 43 is referred to as vactosertib (EW-7197) and compound 44 is referred to as EW-7195.

U.S. application No. 20050096333, describe compounds having Formula IV and are hereby incorporated by reference in its entirety.

Some embodiments of the present disclosure include compounds of Formula IV:

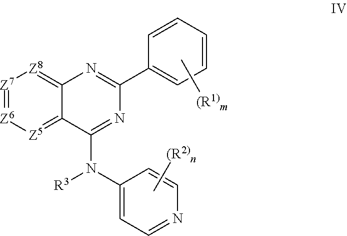

or salts, pharmaceutically acceptable salts, or prodrugs thereof.

An illustrative compound of Formula (IV) is shown in Table 4.

TABLE 4

| | |
|---|---|
| 45 | 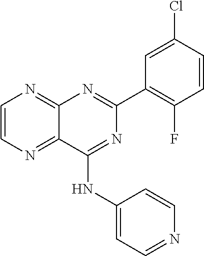 [SMILES: Fc1ccc(Cl)cc1-c1nc(Nc2ccncc2)c2nccnc2n1] |

In some embodiment, compound 45 is referred to as SD208.

World Intellectual Property Organization, WO/2002/066462, describe compounds having Formula V and is hereby incorporated by reference in its entirety.

Some embodiments of the present disclosure include compounds of Formula V:

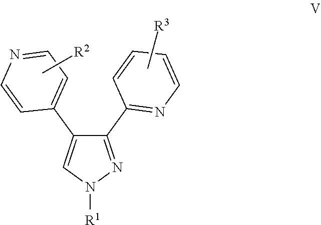

or salts, pharmaceutically acceptable salts, or prodrugs thereof.

An illustrative compound of Formula (V) is shown in Table 5.

TABLE 5

| | |
|---|---|
| 46 | 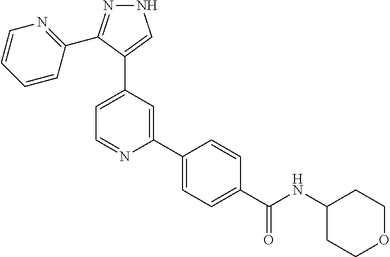 [SMILES: O=C(NC1CCOCC1)c1ccc(-c2cc(-c3c[nH]nc3-c3ccccn3)ccn2)cc1] |

In some embodiment, compound 46 is referred to as GW788388.

Molecular Pharmacology (2019), 95(2), 222-234 describe compounds 47 and 48 shown in Table 6 and are hereby incorporated by reference in their entirety.

TABLE 6

| | |
|---|---|
| 47 | 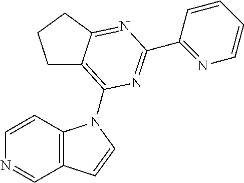 [SMILES: c1ccc(-c2nc3c(c(-n4ccc5cnccc54)n2)CCC3)nc1] |
| 48 | 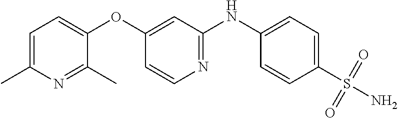 [SMILES: Cc1ccc(Oc2ccnc(Nc3ccc(S(N)(=O)=O)cc3)c2)c(C)n1] |

In some embodiment, compound 47 is referred to as AZ12601011 and compound 48 is referred to as AZ12799734.

U.S. application No. 20160096823, describe compounds having Formula VI and are hereby incorporated by reference in its entirety.

Some embodiments of the present disclosure include compounds of Formula VI:

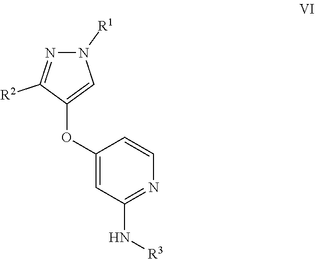

or salts, pharmaceutically acceptable salts, or prodrugs thereof.

An illustrative compound of Formula (VI) is shown in Table 7.

TABLE 7

| | |
|---|---|
| 49 | 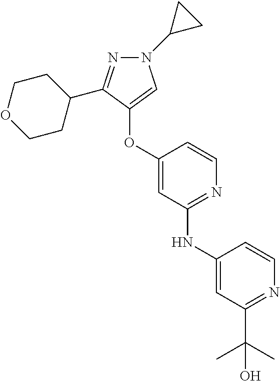 [SMILES: CC(C)(O)c1cc(Nc2cc(Oc3cn(C4CC4)nc3C3CCOCC3)ccn2)ccn1] |

In some embodiment, compound 49 is referred to as LY3200882.

U.S. Pat. No. 8,067,591 and World Intellectual Property Organization, WO/2020/058820A1, describe compounds having Formula VII and are hereby incorporated by reference in their entirety.

Some embodiments of the present disclosure include compounds of Formula VII:

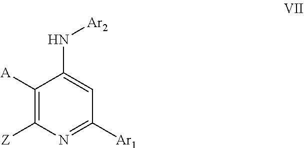

or salts, pharmaceutically acceptable salts, or prodrugs thereof.

An illustrative compound of Formula (VII) is shown in Table 8.

TABLE 8

| | |
|---|---|
| 49 | 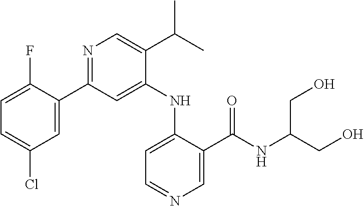 [SMILES: CC(C)c1cnc(-c2cc(Cl)ccc2F)cc1Nc1ccncc1C(=O)NC(CO)CO] |

In some embodiment, compound 49 is referred to as PF-06952229.

### Compound Preparation

The starting materials used in preparing the compounds of the disclosure are known, made by known methods, or are commercially available. It will be apparent to the skilled artisan that methods for preparing precursors and functionality related to the compounds claimed herein are generally described in the literature. The skilled artisan given the literature and this disclosure is well equipped to prepare any of the compounds.

It is recognized that the skilled artisan in the art of organic chemistry can readily carry out manipulations without further direction, that is, it is well within the scope and practice of the skilled artisan to carry out these manipulations. These include reduction of carbonyl compounds to their corresponding alcohols, oxidations, acylations, aromatic substitutions, both electrophilic and nucleophilic, etherifications, esterification and saponification and the like. These manipulations are discussed in standard texts such as March's Advanced Organic Chemistry: Reactions, Mechanisms, and Structure 7^{th} Ed., John Wiley & Sons (2013), Carey and Sundberg, Advanced Organic Chemistry 5^{th} Ed., Springer (2007), Comprehensive Organic Transformations: A Guide to Functional Group Transformations, 2^{nd} Ed., John Wiley & Sons (1999) (incorporated herein by reference in its entirety) and the like.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in P. Wuts Greene's Protective Groups in Organic Synthesis. 5th Ed., John Wiley & Sons (2014), incorporated herein by reference in its entirety.

The following example schemes are provided for the guidance of the reader, and collectively represent an example method for making the compounds provided herein. Furthermore, other methods for preparing compounds of the disclosure will be readily apparent to the person of ordinary skill in the art in light of the following reaction schemes and examples. The skilled artisan is thoroughly equipped to prepare these compounds by those methods given the literature and this disclosure. The compound numberings used in the synthetic schemes depicted below are meant for those specific schemes only and should not be construed as or confused with same numberings in other sections of the application. Unless otherwise indicated, all variables are as defined above.

### General Procedures

General synthetic schemes for preparing compounds of Formula I are set forth below in Scheme 1 and Scheme 2.

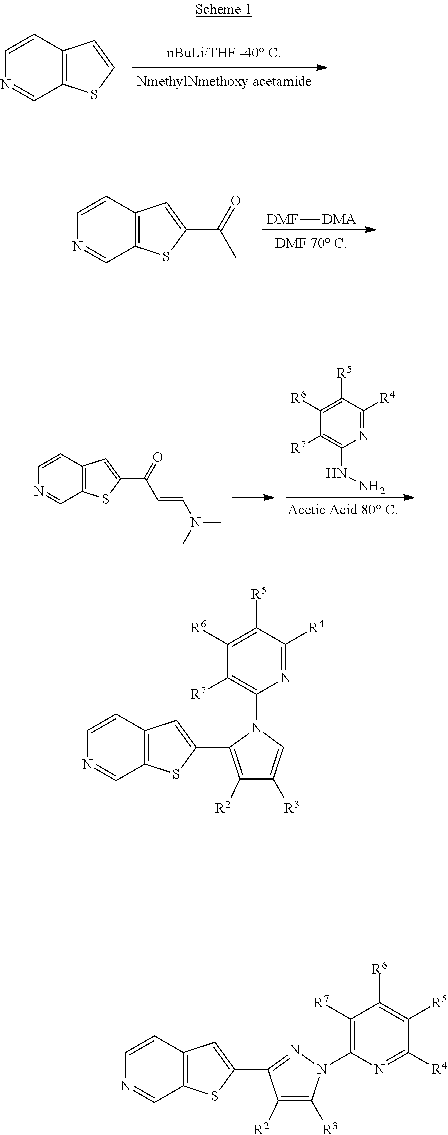

Scheme 1 depicts the synthesis of a pyrazole. A thieno[3,2-c]pyridine 1 (J. Het. Chem. (1993), 30, 289-290) in an aprotic solvent, such as THF (tetrahydrofuran), diethylether, etc. may be reacted with an alkyllithium reagent such as n-butyllithium at or below about −40° C. The thieno[3,2-c]pyridine is shown as unsubstituted in Scheme 1; however, it may be optionally substituted as described herein. Then N-methyl-N-methoxyacetamide (or other suitable acylating agents such as N-acetyl-morpholine, acetic anhydride, and acetyl chloride) is added to the reaction and the reaction is allowed to proceed at −30 to −45° C. to provide the ketone (e.g., 1-(thieno[3,2-c]pyridin-2-yl)ethanone). The ketone is then reacted with dimethylformamide-dimethyl acetal (“DMF-DMA”) in DMF (dimethylformamide) at about 70° C. to provide (e.g., (E)-3-(dimethylamino)-1-(thieno[3,2-c]pyridin-2-yl)prop-2-en-1-one) which is then treated with a pyridinyl-hydrazine (e.g., 1-(6-methylpyridin-2-yl)hydrazine) in acetic acid at about 80° C. to yield the two regioisomers shown. The regioisomer can be separated from the desired compound using conventional purification techniques such as precipitation, filtration, and column chromatography.

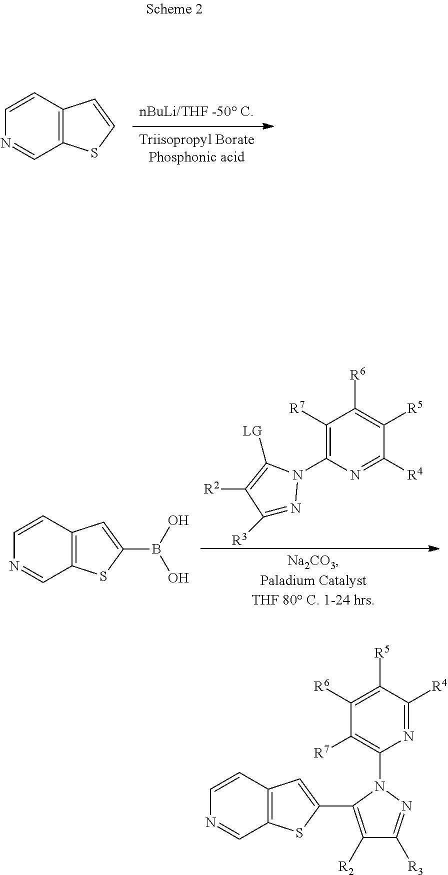

Scheme 2 depicts an alternate synthetic route to the pyrazole. A solution of a thieno[3,2-c]pyridine may be reacted under a nitrogen gas atmosphere, in a solvent such as THE at about-50° C. to −78° C. with an alkyllithium reagent such as n-butyllithium. The thieno[3,2-c]pyridine is shown as unsubstituted in Scheme 2; however, it may be optionally substituted as described herein. The addition of triisopropyl borate and phosphoric acid yields the phosphoric acid salt of the boronic acid. The boronate is then coupled to the pyridinyl-pyrazole to provide the pyrazole, by the addition of a base such as an inorganic carbonate base (e.g., Na₂CO₃, K₂CO₃, NaHCO₃, etc.) or potassium phosphate tribasic, and a palladium catalyst such as Pd(Cl₂)dppf [1,1′-Bis(diphenylphosphino)ferrocene]dichloropalladium(II)]. The reaction may be carried out by refluxing for 1-24 hours in a suitable solvent such as THF or 1,2-dimethoxyethane; or at about 80-100° C. in dioxane. This reaction may also be carried out in the presence of KF and water. The corresponding boronate esters may be used in place of the boronic acid. The group LG represents a suitable leaving group such as trifluoromethanesulfonyl, Br, I, or Cl.

The corresponding thieno[3,2-b]pyridin-2-yl, thieno[2,3-c]pyridin-2-yl, and thieno[2,3-b]pyridin-2-yl analogs may be prepared using thieno[3,2-b]pyridine, thieno[2,3-c]pyridine, and thieno[2,3-b]pyridine, respectively, in place of thieno[3,2-c]pyridine.

### Administration and Pharmaceutical Compositions

Some embodiments include pharmaceutical compositions comprising: (a) a therapeutically effective amount of a compound provided herein, or its corresponding enantiomer, diastereoisomer or tautomer, or pharmaceutically acceptable salt; and (b) a pharmaceutically acceptable carrier.

For purposes of the method described herein, an ALK5 inhibitor compound, most preferably 2-[4-methyl-1-(6-methylpyridin-2-yl)-1H-pyrazol-5-yl]thieno[3,2-c]pyridine (3) may be administered using a liquid nebulization, dry powder or metered-dose inhaler. In some embodiments, ALK5 inhibitor compounds disclosed herein are produced as a pharmaceutical composition suitable for aerosol formation, dose for indication, deposition location, pulmonary or intra-nasal delivery for pulmonary, intranasal/sinus, or extra-respiratory therapeutic action, good taste, manufacturing and storage stability, and patient safety and tolerability.

In some embodiments, the active pharmaceutical ingredient is a salt of an ALK5 inhibitor compound. In some such embodiments, the cation is selected from the group consisting of sodium, potassium, lithium, ammonium, calcium, magnesium, iron, zinc, copper, manganese, aluminum, and beryllium. In some embodiments, the active pharmaceutical ingredient is not a salt of an ALK5 inhibitor compound. In some such embodiments, the composition is a stable, water-soluble formulation.

The ALK5 inhibitor compounds provided herein may also be useful in combination (administered together or sequentially) with other known agents.

In some embodiments, idiopathic pulmonary fibrosis/pulmonary fibrosis can be treated with a combination of a compound of Formula (I) and one or more of the following drugs: pirfenidone (pirfenidone was approved for use in 2011 in Europe under the brand name Esbriet®), prednisone, azathioprine (Imuran®), N-acetylcysteine, interferon-γ 1b, cyclophosphamide (Endoxan®, Cytoxan®, Neosar®, Procytox®, Revimmune®, and Cycloblastin®), mycophenolate mofetil/mycophenolic acid (CellCept®), nintedanib (Ofev® and Vargatef®), Actemra (Tocilizumab), and anti-inflammatory agents such as corticosteroids. In some embodiments, other forms of interstitial lung disease (ILD) can be treated with a combination of a compound of Formula (I) and one or more of the following anti-inflammatory therapies such as methotrexate, cyclophosphamide, cyclosporine, rapamycin (sirolimus), and tacrolimus.

In some embodiments, a compound of Formula (I) can be used to treat idiopathic pulmonary fibrosis/pulmonary fibrosis in combination with any of the following methods: oxygen therapy, pulmonary rehabilitation and surgery.

Administration of the compounds disclosed herein or the pharmaceutically acceptable salts thereof can be via any of the accepted modes of administration, including, but not limited to, oral, intranasal, intrapulmonary, intrabronchial, via inhalation, via endotracheal or endobronchial instillation, via direct instillation into pulmonary cavities, intrathoracically, nose-only aerosol inhalation, intratracheal liquid, spray instillation, dry-powder insufflation, and via thoracostomy irrigation. In some embodiments, the administration method includes via inhalation administration.

The compounds can be administered either alone or in combination with a conventional pharmaceutical carrier, excipient or the like. Pharmaceutical acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Actual methods of preparing dosage forms are known, or will be apparent, to those skilled in this art; for example, see Remington: The Science and Practice of Pharmacy, 22^{nd} Edition (Pharmaceutical Press, London, UK. 2012).

Liquid pharmaceutically administrable compositions can, for example, be prepared by dissolving, dispersing, etc. a compound provided herein and optional pharmaceutical adjuvants in a carrier (e.g., water, saline, aqueous dextrose, glycerol, glycols, ethanol or the like) to form a solution, colloid, liposome, emulsion, complexes, coacervate or suspension. If desired, the pharmaceutical composition can also contain minor amounts of nontoxic auxiliary substances such as wetting agents, emulsifying agents, co-solvents, solubilizing agents, pH buffering agents and the like (e.g., sodium acetate, sodium citrate, cyclodextrin derivatives, sorbitan monolaurate, triethanolamine acetate, triethanolamine oleate, and the like).

In one embodiment, aqueous formulations containing soluble or nanoparticulate drug particles are provided. Aqueous aerosol formulations provide effective delivery to appropriate areas of the lung, with the more concentrated aerosol formulations having the additional advantage of enabling large quantities of drug substance to be delivered to the lung in a very short period of time. In one embodiment, a formulation is optimized to provide a well-tolerated formulation. Accordingly, in one embodiment, ALK5 inhibitor compounds disclosed herein are formulated to have good taste, pH from about 4.0 to about 8.0, osmolarity from about 100 to about 5000 mOsmol/kg. In some embodiments, the osmolarity is from about 100 to about 1000 mOsmol/kg. In some embodiments, the osmolarity is from about 200 to about 500 mOsmol/kg. In some embodiments, the permeant ion concentration is from about 30 to about 300 mM.

In some embodiments, described herein is an aqueous pharmaceutical composition comprising an ALK5 inhibitor compound, water and one or more additional ingredients selected from co-solvents, tonicity agents, sweeteners, surfactants, wetting agents, chelating agents, anti-oxidants, salts, and buffers. It should be understood that many excipients may serve several functions, even within the same formulation.

In some embodiments, pharmaceutical compositions described herein do not include any thickening agents.

In some embodiments, the pH is between about pH 4.0 and about pH 8.0. In some embodiments, the pH is between about pH 5.0 and about pH 8.0. In some embodiments, the pH is between about pH 6.0 and about pH 8.0. In some embodiments, the pH is between about pH 6.5 and about pH 8.0.

In some embodiments, the pharmaceutical composition includes one or more co-solvents. In some embodiments, the pharmaceutical composition includes one or more co-solvents, where the total amount of co-solvents is from about 1% to about 50% v/v of the total volume of the composition. In some embodiments, the pharmaceutical composition includes one or more co-solvents, where the total amount of co-solvents is from about 1% to about 50% v/v, from about 1% to about 40% v/v, from about 1% to about 30% v/v, or from about 1% to about 25% v/v, of the total volume of the composition. Co-solvents include, but are not limited to, ethanol, propylene glycol, glycerol, PEG 200-400. Co-solvent can also include lipid dispersions with oils like Medium Chain Triglycerides (MCT), Glyceryl mono-oleate, Diethyl Sebacate combination with surfactants like lecithins, Polyoxyethylated fatty acids, Poloxamers In some embodiments, the aqueous pharmaceutical composition includes ethanol at about 1% v/v to about 25%. In some embodiments, the aqueous pharmaceutical composition includes ethanol at about 1% v/v to about 15%. In some embodiments, the aqueous pharmaceutical composition includes ethanol at about 1% v/v, 2% v/v, 3% v/v, 4% v/v, 5% v/v, 6% v/v, 7% v/v, 8% v/v, 9% v/v, 10% v/v, 11% v/v, 12% v/v, 13% v/v, 14% v/v, 15% v/v, 16% v/v, 17% v/v, 18% v/v, 19% v/v, 20% v/v, 21% v/v, 22% v/v, 23% v/v, 24% v/v, or 25% v/v. In some embodiments, the aqueous pharmaceutical composition includes glycerol at about 1% v/v to about 25%. In some embodiments, the aqueous pharmaceutical composition includes glycerol at about 1% v/v to about 15%. In some embodiments, the aqueous pharmaceutical composition includes glycerol at about 1% v/v, 2% v/v, 3% v/v, 4% v/v, 5% v/v, 6% v/v, 7% v/v, 8% v/v, 9% v/v, 10% v/v, 11% v/v, 12% v/v, 13% v/v, 14% v/v, 15% v/v, 16% v/v, 17% v/v, 18% v/v, 19% v/v, 20% v/v, 21% v/v, 22% v/v, 23% v/v, 24% v/v, or 25% v/v. In some embodiments, the aqueous pharmaceutical composition includes propylene glycol at about 1% v/v to about 50%. In some embodiments, the aqueous pharmaceutical composition includes propylene glycol at about 1% v/v to about 25%. In some embodiments, the aqueous pharmaceutical composition includes propylene glycol at about 1% v/v, 2% v/v, 3% v/v, 4% v/v, 5% v/v, 6% v/v, 7% v/v, 8% v/v, 9% v/v, 10% v/v, 11% v/v, 12% v/v, 13% v/v, 14% v/v, 15% v/v, 16% v/v, 17% v/v, 18% v/v, 19% v/v, 20% v/v, 21% v/v, 22% v/v, 23% v/v, 24% v/v, or 25% v/v.

In some embodiments, the aqueous pharmaceutical composition includes ethanol at about 1% v/v to about 25% and propylene glycol at about 1% v/v to about 50%. In some embodiments, the aqueous pharmaceutical composition includes ethanol at about 1% v/v to about 15% and propylene glycol at about 1% v/v to about 30%. In some embodiments, the aqueous pharmaceutical composition includes ethanol at about 1% v/v to about 8% and propylene glycol at about 1% v/v to about 16%. In some embodiments, the aqueous pharmaceutical composition includes ethanol and twice as much propylene glycol, based on volume.

In some embodiments, the aqueous pharmaceutical composition includes a buffer. In some embodiments, the buffer is a citrate buffer or a phosphate buffer. In some embodiments, the buffer is a citrate buffer. In some embodiments, the buffer is a phosphate buffer.

In some embodiments, the aqueous pharmaceutical composition consists essentially of an ALK5 inhibitor compound, water, ethanol and/or propylene glycol, a buffer to maintain the pH at about 4 to 8 and optionally one or more ingredients selected from salts, surfactants, and sweeteners (taste-masking agents). In some embodiments, the one or more salts are selected from tonicity agents. In some embodiments, the one or more salts are selected from sodium chloride and magnesium chloride.

In some embodiments, the pharmaceutical composition comprises an ALK5 inhibitor compound at a concentration of about 1 mg/mL to about 50 mg/mL, in a combination of water with one or more cosolvents (e.g., ethanol at a concentration of about 1% v/v to about 10% v/v and/or propylene glycol at a concentration of about 1% v/v to about 50% v/v). Desirably the composition contains a buffer to maintain the pH at about 4 to 8, and optionally one or more ingredients selected from salts, surfactants, and sweeteners (taste-masking agents).

In one embodiment, the solution or diluent used for preparation of aerosol formulations has a pH range from about 4.0 to about 8.0. This pH range improves tolerability.

By non-limiting example, compositions may also include a buffer or a pH adjusting agent, typically a salt prepared from an organic acid or base. Representative buffers include organic acid salts of citric acid, ascorbic acid, gluconic acid, carbonic acid, tartaric acid, succinic acid, acetic acid, or phthalic acid, Tris (also known as tromethamine), hydrochloride, or phosphate buffers.

Many patients have increased sensitivity to various chemical tastes, including bitter, salt, sweet, metallic sensations. To create well-tolerated drug products, by non-limiting example taste masking may be accomplished through the addition of taste-masking excipients, adjusted osmolality, and sweeteners.

In some embodiments, the osmolality of aqueous solutions of the ALK5 inhibitor compound disclosed herein are adjusted by providing excipients. In some cases, a certain amount of chloride or another anion is needed for successful and efficacious delivery of aerosolized the ALK5 inhibitor compound.

ALK5 inhibitor compounds provided herein intended for pharmaceutical use may be administered as crystalline or amorphous products such as lyophilized or amorphous drug or complexes such as spray dried dispersions, or co-crystals. Pharmaceutically acceptable compositions of such solid forms may include liquid, solutions, colloidal, liposomes, emulsions, suspensions, and aerosols. Dosage forms, such as, e.g., powders, liquids, suspensions, aerosols, controlled release or the like. They may be obtained, for example, as solid plugs, powders, or films by methods such as precipitation, crystallization, milling, grinding, supercritical fluid processing, coacervation, complex coacervation, encapsulation, emulsification, complexation, freeze drying, spray drying, or evaporative drying. Microwave or radio frequency drying may be used for this purpose.

Solid compositions can be provided in various different types of dosage forms, depending on the physicochemical properties of the ALK5 inhibitor compound provided herein, the desired dissolution rate, cost considerations, and other criteria. In one of the embodiments, the solid composition is a single unit. This implies that one-unit dose of the compound is comprised in a single, physically shaped solid form or article. In other words, the solid composition is coherent, which is in contrast to a multiple unit dosage form, in which the units are incoherent.

On the other hand, for some applications the solid composition may also be formed as a multiple unit dosage form as defined above. Examples of multiple units are powders, granules, microparticles, pellets, mini-tablets, beads, lyophilized powders, and the like. In one embodiment, the solid composition is a lyophilized powder. Such a dispersed lyophilized system comprises a multitude of powder particles, and due to the lyophilization process used in the formation of the powder, each particle has an irregular, porous microstructure through which the powder is capable of absorbing water very rapidly, resulting in quick dissolution. Effervescent compositions are also contemplated to aid the quick dispersion and absorption of the compound.

Another type of multiparticulate system which is also capable of achieving rapid drug dissolution is that of powders, granules, or pellets from water-soluble excipients which are coated with a compound provided herein so that the compound is located at the outer surface of the individual particles. In this type of system, the water-soluble low molecular weight excipient may be useful for preparing the cores of such coated particles, which can be subsequently coated with a coating composition comprising the compound and, for example, one or more additional excipients, such as a binder, a pore former, a saccharide, a sugar alcohol, a film-forming polymer, a plasticizer, or other excipients used in pharmaceutical coating compositions.

In some embodiments, solid drug nanoparticles are provided for use in generating dry aerosols or for generating nanoparticles in liquid suspension. Powders comprising nanoparticulate drug can be made by spray-drying aqueous dispersions of a nanoparticulate drug and a surface modifier to form a dry powder which consists of aggregated drug nanoparticles. In one embodiment, the aggregates can have a size of about 1 to about 2 microns which is suitable for deep lung delivery. The aggregate particle size can be increased to target alternative delivery sites, such as the upper bronchial region or nasal mucosa by increasing the concentration of drug in the spray-dried dispersion or by increasing the droplet size generated by the spray dryer.

Alternatively, an aqueous dispersion of drug and surface modifier can contain a dissolved diluent such as lactose or mannitol which, when spray dried, forms respirable diluent particles, each of which contains at least one embedded drug nanoparticle and surface modifier. The diluent particles with embedded drug can have a particle size of about 1 to about 2 microns, suitable for deep lung delivery. In addition, the diluent particle size can be increased to target alternate delivery sites, such as the upper bronchial region or nasal mucosa by increasing the concentration of dissolved diluent in the aqueous dispersion prior to spray drying, or by increasing the droplet size generated by the spray dryer.

Spray-dried powders can be used in dry powder inhalers or pressurized metered dose inhalers, either alone or combined with freeze-dried nanoparticulate powder. In addition, spray-dried powders containing drug nanoparticles can be reconstituted and used in either jet or ultrasonic nebulizers to generate aqueous dispersions having respirable droplet sizes, where each droplet contains at least one drug nanoparticle. Concentrated nanoparticulate dispersions may also be used in these embodiments of the disclosure.

Nanoparticulate drug dispersions can also be freeze-dried to obtain powders suitable for nasal or pulmonary delivery. Such powders may contain aggregated nanoparticulate drug particles having a surface modifier. Such aggregates may have sizes within a respirable range, e.g., about 1 to about 5 microns mass median aerodynamic diameter (MMAD).

Freeze dried powders of the appropriate particle size can also be obtained by freeze drying aqueous dispersions of drug and surface modifier, which additionally contain a dissolved diluent such as lactose or mannitol. In these instances, the freeze-dried powders consist of respirable particles of diluent, each of which contains at least one embedded drug nanoparticle.

Freeze-dried powders can be used in dry powder inhalers or pressurized metered dose inhalers, either alone or combined with spray-dried nanoparticulate powder. In addition, freeze-dried powders containing drug nanoparticles can be reconstituted and used in either jet or ultrasonic nebulizers to generate aqueous dispersions that have respirable droplet sizes, where each droplet contains at least one drug nanoparticle.

One embodiment of the disclosure is directed to a process and composition for propellant-based systems comprising nanoparticulate drug particles and a surface modifier. Such formulations may be prepared by wet milling the coarse drug substance and surface modifier in liquid propellant, either at ambient pressure or under high pressure conditions. Alternatively, dry powders containing drug nanoparticles may be prepared by spray-drying or freeze-drying aqueous dispersions of drug nanoparticles and the resultant powders dispersed into suitable propellants for use in conventional pressurized metered dose inhalers. Such nanoparticulate pressurized metered dose inhaler formulations can be used for either nasal or pulmonary delivery. For pulmonary administration, such formulations afford increased delivery to the deep lung regions because of the small (e.g., about 1 to about 2 microns MMAD) particle sizes available from these methods. Concentrated aerosol formulations can also be employed in pressurized metered dose inhalers.

Another embodiment is directed to dry powders which contain nanoparticulate compositions for pulmonary or nasal delivery. The powders may consist of respirable aggregates of nanoparticulate drug particles, or of respirable particles of a diluent which contains at least one embedded drug nanoparticle. Powders containing nanoparticulate drug particles can be prepared from aqueous dispersions of nanoparticles by removing the water via spray-drying or lyophilization (freeze drying). Spray-drying is less time consuming and less expensive than freeze-drying, and therefore more cost-effective. However, certain drugs, such as biologicals benefit from lyophilization rather than spray-drying in making dry powder formulations.

Conventional micronized drug particles used in dry powder aerosol delivery having particle diameters of from about 1 to about 5 microns MMAD are often difficult to meter and disperse in small quantities because of the electrostatic cohesive forces inherent in such powders. These difficulties can lead to loss of drug substance to the delivery device as well as incomplete powder dispersion and sub-optimal delivery to the lung. Since the average particle sizes of conventionally prepared dry powders are usually in the range of from about 1 to about 5 microns MMAD, the fraction of material which actually reaches the alveolar region may be quite small. Thus, delivery of micronized dry powders to the lung, especially the alveolar region, is generally very inefficient because of the properties of the powders themselves.

The dry powder aerosols which contain nanoparticulate drugs can be made smaller than comparable micronized drug substance and, therefore, are appropriate for efficient delivery to the deep lung. Moreover, aggregates of nanoparticulate drugs are spherical in geometry and have good flow properties, thereby aiding in dose metering and deposition of the administered composition in the lung or nasal cavities.

Dry nanoparticulate compositions can be used in both dry powder inhalers or pressurized metered dose inhalers. As used herein, “dry” refers to a composition having less than about 5% water.

In one embodiment, compositions are provided containing nanoparticles which have an effective average particle size of about drug particles in the aerodynamic particle size range of 1-5 μm, which have the potential to reach the lower respiratory tract. By “an effective average particle size of less than about 5 μm, it is meant that at least 50% of the drug particles have a weight average particle size of less than about 5 μm when measured by methods such as light scattering techniques. In some embodiments, at least 70% of the drug particles have an average particle size of less than about 5 μm, in some embodiments, at least 90% of the drug particles have an average particle size of less than about 5 μm, and in some embodiments at least about 95% of the particles have a weight average particle size of less than about 5 μm.

Nanoparticulate drug compositions for aerosol administration can be made by, for example, (1) nebulizing a dispersion of a nanoparticulate drug, obtained by either grinding or precipitation: (2) aerosolizing a dry powder of aggregates of nanoparticulate drug and surface modifier (the aerosolized composition may additionally contain a diluent): or (3) aerosolizing a suspension of nanoparticulate drug or drug aggregates in a non-aqueous propellant. The aggregates of nanoparticulate drug and surface modifier, which may additionally contain a diluent, can be made in a non-pressurized or a pressurized non-aqueous system. Concentrated aerosol formulations may also be made via such methods.

Milling of aqueous drug to obtain nanoparticulate drug may be performed by dispersing drug particles in a liquid dispersion medium and applying mechanical means in the presence of grinding media to reduce the particle size of the drug to the desired effective average particle size. The particles can be reduced in size in the presence of one or more surface modifiers. Alternatively, the particles can be contacted with one or more surface modifiers after attrition. Other compounds, such as a diluent, can be added to the drug/surface modifier composition during the size reduction process. Dispersions can be manufactured continuously or in a batch mode.

Another method of forming nanoparticle dispersion is by microprecipitation. This is a method of preparing stable dispersions of drugs in the presence of one or more surface modifiers and one or more colloid stability enhancing surface active agents free of any trace toxic solvents or solubilized heavy metal impurities. Such a method comprises, for example, (1) dissolving the drug in a suitable solvent with mixing; (2) adding the formulation from step (1) with mixing to a solution comprising at least one surface modifier to form a clear solution; and (3) precipitating the formulation from step (2) with mixing using an appropriate nonsolvent. The method can be followed by removal of any formed salt, if present, by dialysis or diafiltration and concentration of the dispersion by conventional means. The resultant nanoparticulate drug dispersion can be utilized in liquid nebulizers or processed to form a dry powder for use in a dry powder inhaler or pressurized metered dose inhaler.

In a non-aqueous, non-pressurized milling system, a non-aqueous liquid having a vapor pressure of about 1 atm or less at room temperature and in which the drug substance is essentially insoluble may be used as a wet milling medium to make a nanoparticulate drug composition. In such a process, a slurry of drug and surface modifier may be milled in the non-aqueous medium to generate nanoparticulate drug particles. Examples of suitable non-aqueous media include ethanol, trichloromonofluoromethane, (CFC-11), and dichlorotetrafluoroethane (CFC-114). An advantage of using CFC-11 is that it can be handled at only marginally cool room temperatures, whereas CFC-114 requires more controlled conditions to avoid evaporation. Upon completion of milling the liquid medium may be removed and recovered under vacuum or heating, resulting in a dry nanoparticulate composition. The dry composition may then be filled into a suitable container and charged with a final propellant. Exemplary final product propellants, which ideally do not contain chlorinated hydrocarbons, include HFA-134a (tetrafluorocthane) and HFA-227 (heptafluoropropane). While non-chlorinated propellants may be preferred for environmental reasons, chlorinated propellants may also be used in this embodiment of the disclosure.

In a non-aqueous, pressurized milling system, a non-aqueous liquid medium having a vapor pressure significantly greater than 1 atm at room temperature may be used in the milling process to make nanoparticulate drug compositions. If the milling medium is a suitable halogenated hydrocarbon propellant, the resultant dispersion may be filled directly into a suitable pressurized metered dose inhaler container. Alternately, the milling medium can be removed and recovered under vacuum or heating to yield a dry nanoparticulate composition. This composition can then be filled into an appropriate container and charged with a suitable propellant for use in a pressurized metered dose inhaler.

Spray drying is a process used to obtain a powder containing nanoparticulate drug particles following particle size reduction of the drug in a liquid medium. In general, spray-drying may be used when the liquid medium has a vapor pressure of less than about 1 atm at room temperature. A spray-dryer is a device which allows for liquid evaporation and drug powder collection. A liquid sample, either a solution or suspension, is fed into a spray nozzle. The nozzle generates droplets of the sample within a range of about 20 to about 100 microns in diameter which are then transported by a carrier gas into a drying chamber. The carrier gas temperature is typically from about 80° C. to about 200° C. The droplets are subjected to rapid liquid evaporation, leaving behind dry particles which are collected in a special reservoir beneath a cyclone apparatus. Smaller particles in the range down about 1 micron to about 5 microns are also possible.

If the liquid sample consists of an aqueous dispersion of nanoparticles and surface modifier, the collected product will consist of spherical aggregates of the nanoparticulate drug particles. If the liquid sample consists of an aqueous dispersion of nanoparticles in which an inert diluent material was dissolved (such as lactose or mannitol), the collected product will consist of diluent (e.g., lactose or mannitol) particles which contain embedded nanoparticulate drug particles. The final size of the collected product can be controlled and depends on the concentration of nanoparticulate drug and/or diluent in the liquid sample, as well as the droplet size produced by the spray-dryer nozzle. Collected products may be used in conventional dry powder inhalers for pulmonary or nasal delivery, dispersed in propellants for use in pressurized metered dose inhalers, or the particles may be reconstituted in water for use in nebulizers.

In some instances, it may be desirable to add an inert carrier to the spray-dried material to improve the metering properties of the final product. This may especially be the case when the spray dried powder is very small (less than about 5 micron) or when the intended dose is extremely small, whereby dose metering becomes difficult. In general, such carrier particles (also known as bulking agents) are too large to be delivered to the lung and simply impact the mouth and throat and are swallowed. Such carriers typically consist of sugars such as lactose, mannitol, or trehalose. Other inert materials, including polysaccharides and cellulosics, may also be useful as carriers.

Spray-dried powders containing nanoparticulate drug particles may use in conventional dry powder inhalers, dispersed in propellants for use in pressurized metered dose inhalers, or reconstituted in a liquid medium for use with nebulizers.

For compounds that are denatured or destabilized by heat, such as compounds having a low melting point (i.e., about 70° C. to about 150° C.), sublimation is preferred over evaporation to obtain a dry powder nanoparticulate drug composition. This is because sublimation avoids the high process temperatures associated with spray-drying. In addition, sublimation, also known as freeze-drying or lyophilization, can increase the shelf stability of drug compounds. Freeze-dried particles can also be reconstituted and used in nebulizers. Aggregates of freeze-dried nanoparticulate drug particles can be blended with either dry powder intermediates or used alone in dry powder inhalers or pressurized metered dose inhalers for either nasal or pulmonary delivery.

Sublimation involves freezing the product and subjecting the sample to strong vacuum conditions. This allows for the formed ice to be transformed directly from a solid state to a vapor state. Such a process is highly efficient and, therefore, provides greater yields than spray-drying. The resultant freeze-dried product contains drug and modifier(s). The drug is typically present in an aggregated state and can be used for inhalation alone (either pulmonary or nasal), in conjunction with diluent materials (lactose, mannitol, etc.), in dry powder inhalers or pressurized metered dose inhalers, or reconstituted for use in a nebulizer.

In some embodiments, ALK5 inhibitor compounds disclosed herein may be formulated into liposome particles, which can then be aerosolized for inhaled delivery. Lipids which are useful in the present disclosure can be any of a variety of lipids including both neutral lipids and charged lipids. Carrier systems having desirable properties can be prepared using appropriate combinations of lipids, targeting groups and circulation enhancers. Additionally, the compositions provided herein can be in the form of liposomes or lipid particles. As used herein, the term “lipid particle” refers to a lipid bilayer carrier which “coats” a compound and has little or no aqueous interior. More particularly, the term is used to describe a self-assembling lipid bilayer carrier in which a portion of the interior layer comprises cationic lipids which form ionic bonds or ion-pairs with negative charges on the compound (e.g., a plasmid phosphodiester backbone). The interior layer can also comprise neutral or fusogenic lipids and, in some embodiments, negatively charged lipids. The outer layer of the particle will typically comprise mixtures of lipids oriented in a tail-to-tail fashion (as in liposomes) with the hydrophobic tails of the interior layer. The polar head groups present on the lipids of the outer layer will form the external surface of the particle.

Liposomal bioactive agents can be designed to have a sustained therapeutic effect or lower toxicity allowing less frequent administration and an enhanced therapeutic index. Liposomes are composed of bilayers that entrap the desired pharmaceutical. These can be configured as multilamellar vesicles of concentric bilayers with the pharmaceutical trapped within either the lipid of the different layers or the aqueous space between the layers.

By non-limiting example, lipids used in the compositions may be synthetic, semi-synthetic or naturally-occurring lipids, including phospholipids, tocopherols, steroids, fatty acids, glycoproteins such as albumin, negatively-charged lipids and cationic lipids. Phosholipids include egg phosphatidylcholine (EPC), egg phosphatidylglycerol (EPG), egg phosphatidylinositol (EPI), egg phosphatidylserine (EPS), phosphatidylethanolamine (EPE), and egg phosphatidic acid (EPA); the soya counterparts, soy phosphatidylcholine (SPC); SPG, SPS, SPI, SPE, and SPA; the hydrogenated egg and soya counterparts (e.g., HEPC, HSPC), other phospholipids made up of ester linkages of fatty acids in the 2 and 3 of glycerol positions containing chains of 12 to 26 carbon atoms and different head groups in the 1 position of glycerol that include choline, glycerol, inositol, serine, ethanolamine, as well as the corresponding phosphatidic acids. The chains on these fatty acids can be saturated or unsaturated, and the phospholipid can be made up of fatty acids of different chain lengths and different degrees of unsaturation. In particular, the compositions of the formulations can include dipalmitoylphosphatidylcholine (DPPC), a major constituent of naturally-occurring lung surfactant as well dioleoylphosphatidylcholine (DOPC) and dioleoylphosphatidylglycerol (DOPG). Other examples include dimyristoylphosphatidycholine (DMPC) and dimyristoylphosphatidylglycerol (DMPG) dipalmitoylphosphatidcholine (DPPC) and dipalmitoylphosphatidylglycerol (DPPG) distearoylphosphatidylcholine (DSPC) and distearoylphosphatidylglycerol (DSPG), diolcylphosphatidylethanolamine (DOPE) and mixed phospholipids like palmitoylstearoylphosphatidylcholine (PSPC) and palmitoylstearoylphosphatidylglycerol (PSPG), and single acylated phospholipids like mono-oleoyl-phosphatidylethanolamine (MOPE).

In a preferred embodiment, PEG-modified lipids are incorporated into the compositions of the present disclosure as the aggregation-preventing agent. The use of a PEG-modified lipid positions bulky PEG groups on the surface of the liposome or lipid carrier and prevents binding of DNA to the outside of the carrier (thereby inhibiting cross-linking and aggregation of the lipid carrier). The use of a PEG-ceramide is often preferred and has the additional advantages of stabilizing membrane bilayers and lengthening circulation lifetimes. Additionally. PEG-ceramides can be prepared with different lipid tail lengths to control the lifetime of the PEG-ceramide in the lipid bilayer. In this manner. “programmable” release can be accomplished which results in the control of lipid carrier fusion. For example, PEG-ceramides having C20-acyl groups attached to the ceramide moiety will diffuse out of a lipid bilayer carrier with a half-life of 22 hours. PEG-ceramides having C14- and C8-acyl groups will diffuse out of the same carrier with half-lives of 10 minutes and less than 1 minute, respectively. As a result, selection of lipid tail length provides a composition in which the bilayer becomes destabilized (and thus fusogenic) at a known rate. Other PEG-lipids or lipid-polyoxyethylene conjugates are useful in the present compositions. Examples of suitable PEG-modified lipids include PEG-modified phosphatidylethanolamine and phosphatidic acid, PEG-modified diacylglycerols and dialkylglycerols, PEG-modified dialkylamines and PEG-modified 1,2-diacyloxypropan-3-amines. Particularly preferred are PEG-ceramide conjugates (e.g., PEG-Cer-C8, PEG-Cer-C14 or PEG-Cer-C20) which are described in U.S. Pat. No. 5,820,873, incorporated herein by reference.

The compositions of the present disclosure can be prepared to provide liposome compositions which are about 50 nm to about 400 nm in diameter. One skilled in the art will understand that the size of the compositions can be larger or smaller depending upon the volume which is encapsulated. Thus, for larger volumes, the size distribution will typically be from about 80 nm to about 300 nm.

ALK5 inhibitor compounds disclosed herein may be prepared in a pharmaceutical composition with suitable surface modifiers which may be selected from known organic and inorganic pharmaceutical excipients. Such excipients include low molecular weight oligomers, polymers, surfactants and natural products. Preferred surface modifiers include nonionic and ionic surfactants. Two or more surface modifiers can be used in combination.

Representative examples of surface modifiers include cetyl pyridinium chloride, gelatin, casein, lecithin (phosphatides), dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens™, such as e.g., Tween 20™, and Tween 80™, (ICI Specialty Chemicals)): polyethylene glycols (e.g., Carbowaxs 3350™, and 1450™, and Carbopol 934™, (Union Carbide)), dodecyl trimethyl ammonium bromide, polyoxyethylenestearates, colloidal silicon dioxide, phosphates, sodium dodecylsulfate, carboxymethylcellulose calcium, hydroxypropyl cellulose (HPC, HPC-SL, and HPC-L), hydroxypropyl methylcellulose (HPMC), carboxymethylcellulose sodium, methylcellulose, hydroxyethylcellulose, hydroxypropylcellulose, hydroxypropylmethyl-cellulose phthalate, noncrystalline cellulose, magnesium aluminum silicate, triethanolamine, polyvinyl alcohol (PVA), polyvinylpyrrolidone (PVP), 4-(1,1,3,3-tetaamethylbutyl)-phenol polymer with ethylene oxide and formaldehyde (also known as tyloxapol, superione, and triton), poloxamers (e.g., Pluronics F68™, and F108™, which are block copolymers of ethylene oxide and propylene oxide): poloxamines (e.g., Tetronic 908™, also known as Poloxamine 908™, which is a tetrafunctional block copolymer derived from sequential addition of propylene oxide and ethylene oxide to ethylenediamine (BASF Wyandotte Corporation, Parsippany, N.J.)): a charged phospholipid such as dimyristoyl phophatidyl glycerol, dioctylsulfosuccinate (DOSS): Tetronic 1508™; (T-1508) (BASF Wyandotte Corporation), dialkylesters of sodium sulfosuccinic acid (e.g., Acrosol OT™, which is a dioctyl ester of sodium sulfosuccinic acid (American Cyanamid)): Duponol P™, which is a sodium lauryl sulfate (DuPont): Tritons X-200™, which is an alkyl aryl polyether sulfonate (Rohm and Haas); Crodestas F-110™, which is a mixture of sucrose stearate and sucrose distearate (Croda Inc.); p-isononylphenoxypoly-(glycidol), also known as Olin-log™, or Surfactant 10-G™, (Olin Chemicals, Stamford, Conn.); Crodestas SL-40™, (Croda, Inc.); and SA9OHCO, which is C₁₈H₃₇CH₂(CON(CH₃)—CH₂(CHOH)₄(CH₂OH)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; and the like. In some embodiment, the surface modifier is Tyloxapol which is preferred for the pulmonary or intranasal delivery of steroids, even more so for nebulization therapies.

Examples of surfactants for use in the solutions disclosed herein include, but are not limited to, ammonium laureth sulfate, cetamine oxide, cetrimonium chloride, cetyl alcohol, cetyl myristate, cetyl palmitate, cocamide DEA, cocamidopropyl betaine, cocamidopropylamine oxide, cocamide MEA, DEA lauryl sulfate, di-stearyl phthalic acid amide, dicetyl dimethyl ammonium chloride, dipalmitoylethyl hydroxethylmonium, disodium laureth sulfosuccinate, di(hydrogenated) tallow phthalic acid, glyceryl dilaurate, glyceryl distearate, glyceryl oleate, glyceryl stearate, isopropyl myristate nf, isopropyl palmitate nf, lauramide DEA, lauramide MEA, lauramide oxide, myristamine oxide, octyl isononanoate, octyl palmitate, octyldodecyl neopentanoate, olealkonium chloride, PEG-2 stearate, PEG-32 glyceryl caprylate/caprate, PEG-32 glyceryl stearate, PEG-4 and PEG-150 stearate & distearate, PEG-4 to PEG-150 laurate & dilaurate, PEG-4 to PEG-150 oleate & diolcate, PEG-7 glyceryl cocoate, PEG-8 beeswax, propylene glycol stearate, sodium C14-16 olefin sulfonate, sodium lauryl sulfoacetate, sodium lauryl sulphate, sodium trideceth sulfate, stearalkonium chloride, stearamide oxide, TEA-dodecylbenzene sulfonate, TEA lauryl sulfate

Most of these surface modifiers are known pharmaceutical excipients and are described in detail in the Handbook of Pharmaceutical Excipients, published jointly by the American Pharmaceutical Association and The Pharmaceutical Society of Great Britain (The Pharmaceutical Press, 1986), specifically incorporated by reference. The surface modifiers are commercially available and/or can be prepared by techniques known in the art. The relative amount of drug and surface modifier can vary widely and the optimal amount of the surface modifier can depend upon, for example, the particular drug and surface modifier selected, the critical micelle concentration of the surface modifier if it forms micelles, the hydrophilic-lipophilic-balance (HLB) of the surface modifier, the melting point of the surface modifier, the water solubility of the surface modifier and/or drug, the surface tension of water solutions of the surface modifier, etc.

In the present disclosure, the optimal ratio of drug to surface modifier is about 0.1% to about 99.9% ALK5 inhibitor compound. In some embodiments, about 10% to about 90%.

In some embodiments, microspheres can be used for pulmonary delivery of ALK5 inhibitor compounds by first adding an appropriate amount of drug compound to be solubilized in water. For example, an aqueous ALK5 inhibitor compound solution may be dispersed in methylene chloride containing a predetermined amount (0.1-1% w/v) of poly(DL-lactide-co-glycolide) (PLGA) by probe sonication for 1-3 min on an ice bath. Separately, an ALK5 inhibitor compound may be solubilized in methylene chloride containing PLGA (0.1-1% w/v). The resulting water-in-oil primary emulsion or the polymer/drug solution will be dispersed in an aqueous continuous phase consisting of 1-2% polyvinyl alcohol (previously cooled to 4° C.) by probe sonication for 3-5 min on an ice bath. The resulting emulsion will be stirred continuously for 2-4 hours at room temperature to evaporate methylene chloride. Microparticles thus formed will be separated from the continuous phase by centrifuging at 8000-10000 rpm for 5-10 min. Sedimented particles will be washed thrice with distilled water and freeze dried. Freeze-dried ALK5 inhibitor compound microparticles will be stored at −20° C.

In some embodiments, a spray drying approach can be used to prepare ALK5 inhibitor compound microspheres. An appropriate amount of ALK5 inhibitor compound will be solubilized in methylene chloride containing PLGA (0.1-1%). This solution will be spray dried to obtain the microspheres.

In some embodiments, ALK5 inhibitor compound microparticles can be characterized for size distribution (requirement: 90%<5 μm, 95%<10 μm), shape, drug loading efficiency and drug release using appropriate techniques and methods.

In some embodiment, this approach may also be used to sequester and improve the water solubility of solid, AUC shape-enhancing formulations, such as low-solubility ALK5 inhibitor compounds or salt forms for nanoparticle-based formulations.

In some embodiments, an ALK5 inhibitor compound can be first dissolved in the minimal quantity of ethanol 96% necessary to maintain the compound in solution when diluted with water from 96 to 75%. This solution can then be diluted with water to obtain a 75% ethanol solution and then a certain amount of polymer can be added to obtain the following w/w drug/polymer ratios: 1:2, 1:1, 2:1, 3:1, 4:1, 6:1, 9:1, and 19:1. These final solutions are spray-dried under the following conditions; feed rate, 15 mL/min; inlet temperature, 110° C.; outlet temperature, 85° C.; pressure 4 bar and throughput of drying air, 35 m3/hr. Powder is then collected and stored under vacuum in a desiccator.

In some embodiments, preparation of ALK5 inhibitor compound solid lipid particles may involve dissolving the drug in a lipid melt (phospholipids such as phophatidyl choline and phosphatidyl serine) maintained at least at the melting temperature of the lipid, followed by dispersion of the drug-containing melt in a hot aqueous surfactant solution (typically 1-5% w/v) maintained at least at the melting temperature of the lipid. The coarse dispersion will be homogenized for 1-10 min using a Microfluidizer® to obtain a nanoemulsion. Cooling the nanoemulsion to a temperature between 4-25° C. will re-solidify the lipid, leading to formation of solid lipid nanoparticles. Optimization of formulation parameters (type of lipid matrix, surfactant concentration and production parameters) will be performed so as to achieve a prolonged drug delivery. By non-limiting example, this approach may also be used to sequester and improve the water solubility of solid, AUC shape-enhancing formulations, such as low-solubility ALK5 inhibitor compounds or salt forms for nanoparticle-based formulations.

In some embodiments, Melt-Extrusion AUC shape-enhancing ALK5 inhibitor compound formulations may be preparation by dissolving the drugs in micelles by adding surfactants or preparing micro-emulsion, forming inclusion complexes with other molecules such as cyclodextrins, forming nanoparticles of the drugs, or embedding the amorphous drugs in a polymer matrix. Embedding the drug homogeneously in a polymer matrix produces a solid dispersion. Solid dispersions can be prepared in two ways: the solvent method and the hot melt method. The solvent method uses an organic solvent wherein the drug and appropriate polymer are dissolved and then (spray) dried. The major drawbacks of this method are the use of organic solvents and the batch mode production process. The hot melt method uses heat in order to disperse or dissolve the drug in an appropriate polymer. The melt-extrusion process is an optimized version of the hot melt method. The advantage of the melt-extrusion approach is lack of organic solvent and continuous production process. As the melt-extrusion is a novel pharmaceutical technique, the literature dealing with it is limited. The technical set-up involves a mixture and extrusion of ALK5 inhibitor compound, hydroxypropyl-b-cyclodextrin (HP-b-CD), and hydroxypropylmethylcellulose (HPMC), in order to, by non-limiting example create an AUC shape-enhancing formulation of ALK5 inhibitor compound. Cyclodextrin is a toroidal-shaped molecule with hydroxyl groups on the outer surface and a cavity in the center. Cyclodextrin sequesters the drug by forming an inclusion complex. The complex formation between cyclodextrins and drugs has been investigated extensively. It is known that water-soluble polymer interacts with cyclodextrin and drug in the course of complex formation to form a stabilized complex of drug and cyclodextrin co-complexed with the polymer. This complex is more stable than the classic cyclodextrin-drug complex. As one example, HPMC is water soluble; hence using this polymer with HP-b-CD in the melt is expected to create an aqueous soluble AUC shape-enhancing formulation. By non-limiting example, this approach may also be used to sequester and improve the water solubility of solid, AUC shape-enhancing formulations, such as low-solubility ALK5 inhibitor compounds or salt forms for nanoparticle-based formulations.

In some embodiments, co-precipitate ALK5 inhibitor compound formulations may be prepared by formation of co-precipitates with pharmacologically inert, polymeric materials. It has been demonstrated that the formation of molecular solid dispersions or co-precipitates to create an AUC shape-enhancing formulation with various water-soluble polymers can significantly slow their in vitro dissolution rates and/or in vivo absorption. In preparing powdered products, grinding is generally used for reducing particle size, since the dissolution rate is strongly affected by particle size. Moreover, a strong force (such as grinding) may increase the surface energy and cause distortion of the crystal lattice as well as reducing particle size. Co-grinding drug with hydroxypropylmethylcellulose, b-cyclodextrin, chitin and chitosan, crystalline cellulose, and gelatin, may enhance the dissolution properties such that AUC shape-enhancement is obtained for otherwise readily bioavailable ALK5 inhibitor compounds. By non-limiting example, this approach may also be used to sequester and improve the water solubility of solid, AUC shape-enhancing formulations, such as low-solubility ALK5 inhibitor compounds or salt forms for nanoparticle-based formulations.

In some embodiments, compositions may include one or more diketopiperazines, diketomorpholines and diketodioxanes and their substitution analogs. By further non-limiting example, U.S. Pat. No. 10,912,821 disclosing the formation of diketopiperazine carboxylate salts and microparticles containing the same, is hereby incorporated by reference in its entirety.

In some embodiments, an ALK5 inhibitor compound can be incorporated into microparticles formed by heterocyclic compounds. These heterocyclic compounds include, without limitation, diketopiperazines, diketomorpholines and diketodioxanes and their substitution analogs. These heterocyclic compositions comprise rigid hexagonal rings with opposing heteroatoms and unbonded electron pairs. These heterocyclic compounds form microparticles that incorporate the ALK5 inhibitor compound to be delivered. These microparticles include microcapsules, which have an outer shell composed of either the heterocyclic compound alone or in combination with one or more drug(s). The drug may be molecularly dispersed and complexed with the heterocyclic compound matrix or may exist as microcrystalline solids dispersed in the matrix or form a co-crystal with the matrix heterocyclic compounds depending on what the design of the solid is intended to do. It may be designed for deep lung penetration and rapid dissolution on contact with the respiratory membranes; alternatively, the design could be for slow release from the matrix to control the biological response or impact permeability of the membrane in some way as the combination. This outer shell may surround a core material. This outer shell may also surround or constitute microspheres that are either solid or hollow, or a combination thereof, which contain one or more drugs dispersed throughout the sphere and/or adsorbed onto the surface of the sphere. The outer shell also may surround microparticles having irregular shape, either alone or in combination with the aforementioned microspheres. In a preferred embodiment for pulmonary delivery, the microparticles are from about 0.1 microns to about ten microns in diameter. Within drug delivery systems, these microparticles exhibit desirable shapes, density and size distributions as well as good cargo tolerance.

In some embodiments, the heterocyclic compound is a diketopiperazine.

In some embodiments, the diketopiperazine is a derivative of 3,6-di(4-aminobutyl)-2,5-diketopiperazine. Exemplary derivatives include 3,6-di(succinyl-4-aminobutyl)-(succinyl diketopiperazine or SDKP), 3,6-di(maleyl-4-aminobutyl)-, 3,6-di(citraconyl-4-aminobutyl)-, 3,6-di(glutaryl-4-aminobutyl)-, 3,6-di(malonyl-4-aminobutyl)-, 3,6-di(oxalyl-4-aminobutyl)-, and 3,6-di(fumaryl-4-aminobutyl)-2,5-diketopiperazine (fumaryl diketopiperazine or FDKP).

In some embodiments, salts of the diketopiperazine are selected from the group consisting of sodium (Na), potassium (K), lithium (Li), magnesium (Mg), calcium (Ca), ammonium, or mono-, di- or tri-alkylammonium (as derived from triethylamine, butylamine, diethanolamine, triethanolamine, or pyridines, and the like) salts. The salt may be a mono-, di-, or mixed salt. Diketopiperazine salt counter cations may be selected to produce salts having varying solubilities. These varying solubilities can be the result of differences in dissolution rate and/or intrinsic solubility. By controlling the rate of diketopiperazine salt dissolution, the rate of drug absorption from the diketopiperazine salt/ALK5 inhibitor compound combination can also be controlled to provide formulations having immediate and/or sustained release profiles. For example, sodium salts of organic compounds are characteristically highly soluble in biological systems, while calcium salts are characteristically only slightly soluble in biological systems. Thus, a formulation comprised of a diketopiperazine sodium salt/ALK5 inhibitor compound combination would provide immediate drug absorption, while a formulation comprised of a diketopiperazine calcium salt/ALK5 inhibitor compound combination would provide slower drug absorption. Also more basic ALK5 inhibitor compounds can act as salt formers with diketopiperazine acids to form an ionic complex that releases due to solubility and displacement with ions in biological fluid creating a controlling release mechanism. A formulation containing a combination of both of the latter formulations could be used to provide immediate drug absorption followed by a period of sustained absorption.

In some embodiments, compositions may include one or more di- or tripeptides containing two or more leucine residues. By further non-limiting example, U.S. Pat. No. 6,835,372 disclosing dispersion-enhancing peptides, is hereby incorporated by reference in its entirety. This patent describes the discovery that di-leucyl-containing dipeptides (e.g., dileucine) and tripeptides are superior in their ability to increase the dispersibility of powdered composition.

In another embodiment, highly dispersible particles including an amino acid are administered. Hydrophobic amino acids are preferred. Suitable amino acids include naturally occurring and non-naturally occurring hydrophobic amino acids. Some naturally occurring hydrophobic amino acids, including but not limited to, non-naturally occurring amino acids include, for example, beta-amino acids. Both D, L and racemic configurations of hydrophobic amino acids can be employed. Suitable hydrophobic amino acids can also include amino acid analogs. As used herein, an amino acid analog includes the D or L configuration of an amino acid having the following formula; —NH—CHR—CO—, wherein R is an aliphatic group, a substituted aliphatic group, a benzyl group, a substituted benzyl group, an aromatic group or a substituted aromatic group and wherein R does not correspond to the side chain of a naturally-occurring amino acid. As used herein, aliphatic groups include straight chained, branched or cyclic C1-C8 hydrocarbons which are completely saturated, which contain one or two heteroatoms such as nitrogen, oxygen or sulfur and/or which contain one or more units of desaturation. Aromatic groups include carbocyclic aromatic groups such as phenyl and naphthyl and heterocyclic aromatic groups such as imidazolyl, indolyl, thienyl, furanyl, pyridyl, pyranyl, oxazolyl, benzothienyl, benzofuranyl, quinolinyl, isoquinolinyl and acridintyl.

Suitable substituents on an aliphatic, aromatic or benzyl group include-OH, halogen (—Br, —Cl, —I and —F)—O (aliphatic, substituted aliphatic, benzyl, substituted benzyl, aryl or substituted aryl group), —CN, —NO₂, —CO₂H, —NH₂, —NH (aliphatic group, substituted aliphatic, benzyl, substituted benzyl, aryl or substituted aryl group), —N(aliphatic group, substituted aliphatic, benzyl, substituted benzyl, aryl or substituted aryl group)₂, —CO₂ (aliphatic group, substituted aliphatic, benzyl, substituted benzyl, aryl or substituted aryl group), —CONH₂, —CONH (aliphatic, substituted aliphatic group, benzyl, substituted benzyl, aryl or substituted aryl group)), —SH, —S (aliphatic, substituted aliphatic, benzyl, substituted benzyl, aromatic or substituted aromatic group) and —NH—C(CONH)—NH₂. A substituted benzylic or aromatic group can also have an aliphatic or substituted aliphatic group as a substituent. A substituted aliphatic group can also have a benzyl, substituted benzyl, aryl or substituted aryl group as a substituent. A substituted aliphatic, substituted aromatic or substituted benzyl group can have one or more substituents. Modifying an amino acid substituent can increase, for example, the lipophilicity or hydrophobicity of natural amino acids which are hydrophilic.

A number of the suitable amino acids, amino acids analogs and salts thereof can be obtained commercially. Others can be synthesized by methods known in the art.

Hydrophobicity is generally defined with respect to the partition of an amino acid between a nonpolar solvent and water. Hydrophobic amino acids are those acids which show a preference for the nonpolar solvent. Relative hydrophobicity of amino acids can be expressed on a hydrophobicity scale on which glycine has the value 0.5. On such a scale, amino acids which have a preference for water have values below 0.5 and those that have a preference for nonpolar solvents have a value above 0.5. As used herein, the term hydrophobic amino acid refers to an amino acid that, on the hydrophobicity scale, has a value greater or equal to 0.5, in other words, has a tendency to partition in the nonpolar acid which is at least equal to that of glycine.

Examples of amino acids which can be employed include, but are not limited to: glycine, proline, alanine, cysteine, methionine, valine, leucine, tyrosine, isoleucine, phenylalanine, tryptophan. Preferred hydrophobic amino acids include leucine, isoleucine, alanine, valine, phenylalanine and glycine. Combinations of hydrophobic amino acids can also be employed. Furthermore, combinations of hydrophobic and hydrophilic (preferentially partitioning in water) amino acids, where the overall combination is hydrophobic, can also be employed.

The amino acid can be present in the particles of the disclosure in an amount of at least 10 weight %. Preferably, the amino acid can be present in the particles in an amount ranging from about 20 to about 80 weight %. The salt of a hydrophobic amino acid can be present in the particles of the disclosure in an amount of at least 10 weight percent. Preferably, the amino acid salt is present in the particles in an amount ranging from about 20 to about 80 weight %. In preferred embodiments the particles have a tap density of less than about 0.4 g/cm³.

Methods of forming and delivering particles which include an amino acid are described in U.S. Pat. No. 6,586,008, entitled Use of Simple Amino Acids to Form Porous Particles During Spray Drying, the teachings of which are incorporated herein by reference in their entirety.

Protein excipients may include albumins such as human serum albumin (HSA), recombinant human albumin (rHA), gelatin, casein, hemoglobin, and the like. Suitable amino acids (outside of the dileucyl-peptides of the disclosure), which may also function in a buffering capacity, include alanine, glycine, arginine, betaine, histidine, glutamic acid, aspartic acid, cysteine, lysine, leucine, isoleucine, valine, methionine, phenylalanine, aspartame, tyrosine, tryptophan, and the like. Preferred are amino acids and polypeptides that function as dispersing agents. Amino acids falling into this category include hydrophobic amino acids such as leucine, valine, isoleucine, tryptophan, alanine, methionine, phenylalanine, tyrosine, histidine, and proline. Dispersibility-enhancing peptide excipients include dimers, trimers, tetramers, and pentamers comprising one or more hydrophobic amino acid components such as those described above.

By non-limiting example, carbohydrate excipients may include monosaccharides such as fructose, maltose, galactose, glucose, D-mannose, sorbose, and the like; disaccharides, such as lactose, sucrose, trehalose, cellobiose, and the like: polysaccharides, such as raffinose, melezitose, maltodextrins, dextrans, starches, and the like; and alditols, such as mannitol, xylitol, maltitol, lactitol, xylitol sorbitol (glucitol), pyranosyl sorbitol, myoinositol, isomalt, trehalose and the like.

By non-limiting example, compositions may also include polymeric excipients/additives, e.g., polyvinylpyrrolidones, derivatized celluloses such as hydroxymethylcellulose, hydroxyethylcellulose, and hydroxypropylmethylcellulose, Ficolls (a polymeric sugar), hydroxyethylstarch, dextrates (by non-limiting example cyclodextrins may include, 2-hydroxypropyl-beta-cyclodextrin, 2-hydroxypropyl-gamma-cyclodextrin, randomly methylated beta-cyclodextrin, dimethyl-alpha-cyclodextrin, dimethyl-beta-cyclodextrin, maltosyl-alpha-cyclodextrin, glucosyl-1-alpha-cyclodextrin, glucosyl-2-alpha-cyclodextrin, alpha-cyclodextrin, beta-cyclodextrin, gamma-cyclodextrin, and sulfobutylether-β-cyclodextrin), polyethylene glycols, and pectin may also be used.

Highly dispersible particles administered comprise a bioactive agent and a biocompatible, and preferably biodegradable polymer, copolymer, or blend. The polymers may be tailored to optimize different characteristics of the particle including: i) interactions between the agent to be delivered and the polymer to provide stabilization of the agent and retention of activity upon delivery: ii) rate of polymer degradation and, thereby, rate of drug release profiles: iii) surface characteristics and targeting capabilities via chemical modification; and iv) particle porosity.

Surface eroding polymers such as polyanhydrides may be used to form the particles. For example, polyanhydrides such as poly[(p-carboxyphenoxy)hexane anhydride] (PCPH) may be used. Biodegradable polyanhydrides are described in U.S. Pat. No. 4,857,311. Bulk eroding polymers such as those based on polyesters including poly(hydroxy acids) also can be used. For example, polyglycolic acid (PGA), polylactic acid (PLA), or copolymers thereof may be used to form the particles. The polyester may also have a charged or functionalizable group, such as an amino acid. In a preferred embodiment, particles with controlled release properties can be formed of poly(D,L-lactic acid) and/or poly(DL-lactic-co-glycolic acid) (“PLGA”) which incorporate a surfactant such as dipalmitoyl phosphatidylcholine (DPPC).

Other polymers include polyamides, polycarbonates, polyalkylenes such as polyethylene, polypropylene, poly(ethylene glycol), poly(ethylene oxide), poly(ethylene terephthalate), poly vinyl compounds such as polyvinyl alcohols, polyvinyl ethers, and polyvinyl esters, polymers of acrylic and methacrylic acids, celluloses and other polysaccharides, and peptides or proteins, or copolymers or blends thereof. Polymers may be selected with or modified to have the appropriate stability and degradation rates in vivo for different controlled drug delivery applications.

Highly dispersible particles can be formed from functionalized polyester graft copolymers, as described in Hrkach et al., Macromolecules, 28: 4736-4739 (1995); and Hrkach et al., “Poly(L-Lactic acid-co-amino acid) Graft Copolymers: A Class of Functional Biodegradable Biomaterials” in Hydrogels and Biodegradable Polymers for Bioapplications, ACS Symposium Series No. 627, Raphael M, Ottenbrite et al., Eds., American Chemical Society, Chapter 8, pp. 93-101, 1996.

In one embodiment, highly dispersible particles including a bioactive agent and a phospholipid are administered. Examples of suitable phospholipids include, among others, phosphatidylcholines, phosphatidylethanolamines, phosphatidylglycerols, phosphatidylserines, phosphatidylinositols and combinations thereof. Specific examples of phospholipids include but are not limited to phosphatidylcholines dipalmitoyl phosphatidylcholine (DPPC), dipalmitoyl phosphatidylethanolamine (DPPE), distearoyl phosphatidyicholine (DSPC), dipalmitoyl phosphatidyl glycerol (DPPG) or any combination thereof. Other phospholipids are known to those skilled in the art. In another embodiment, the phospholipids are endogenous to the lung.

The phospholipid, can be present in the particles in an amount ranging from about 0 to about 90 weight %. In another embodiment, it can be present in the particles in an amount ranging from about 10 to about 60 weight %.

In another embodiment of the disclosure, the phospholipids or combinations thereof are selected to impart controlled release properties to the highly dispersible particles. The phase transition temperature of a specific phospholipid can be below, about or above the physiological body temperature of a patient. Preferred phase transition temperatures range from 30° C. to 50° C. (e.g., within +/−10° C. of the normal body temperature of patient). By selecting phospholipids or combinations of phospholipids according to their phase transition temperature, the particles can be tailored to have controlled release properties. For example, by administering particles which include a phospholipid or combination of phospholipids which have a phase transition temperature higher than the patient's body temperature, the release of dopamine precursor, agonist or any combination of precursors and/or agonists can be slowed down. On the other hand, rapid release can be obtained by including in the particle's phospholipids having lower transition temperatures.

In some embodiments, ALK5 inhibitor compound formulations disclosed herein and related compositions, may further include one or more taste-masking agents such as flavoring agents, inorganic salts (e.g., sodium chloride), sweeteners, antioxidants, antistatic agents, surfactants (e.g., polysorbates such as “TWEEN 20” and “TWEEN 80”), sorbitan esters, saccharin (e.g., sodium saccharin or other saccharin forms, which as noted elsewhere herein may be present in certain embodiments at specific concentrations or at specific molar ratios relative to an ALK5 inhibitor compound), bicarbonate, cyclodextrins, lipids (e.g., phospholipids such as lecithin and other phosphatidylcholines, phosphatidylethanolamines), fatty acids and fatty esters, steroids (e.g., cholesterol), and chelating agents (e.g., EDTA, zinc and other such suitable cations). Other pharmaceutical excipients and/or additives suitable for use in the compositions according to the disclosure are listed in “Remington: The Science & Practice of Pharmacy”, 19th ed., Williams & Williams, (1995), and in the “Physician's Desk Reference”, 52nd ed., Medical Economics, Montvale, N.J. (1998).

In some embodiment, taste-masking agents in ALK5 inhibitor compound formulations, may include the use of flavorings, sweeteners, and other various coating strategies, for instance, sugars such as sucrose, dextrose, and lactose, carboxylic acids, menthol, amino acids or amino acid derivatives such as arginine, lysine, and monosodium glutamate, and/or synthetic flavor oils and flavoring aromatics and/or natural oils, extracts from plants, leaves, flowers, fruits, etc. and combinations thereof. These may include cinnamon oils, oil of wintergreen, peppermint oils, clover oil, bay oil, anise oil, eucalyptus, vanilla, citrus oil such as lemon oil, orange oil, grape and grapefruit oil, fruit essences including apple, peach, pear, strawberry, raspberry, cherry, plum, pineapple, apricot, etc. Additional sweeteners include sucrose, dextrose, aspartame (NutraSweet R®), acesulfame-K, sucralose and saccharin (e.g., sodium saccharin or other saccharin forms, which as noted elsewhere herein may be present in certain embodiments at specific concentrations or at specific molar ratios relative to an ALK5 inhibitor compound), organic acids (by non-limiting example citric acid and aspartic acid). Such flavors may be present at from about 0.05 to about 4 percent by weight, and may be present at lower or higher amounts as a factor of one or more of potency of the effect on flavor, solubility of the flavorant, effects of the flavorant on solubility or other physicochemical or pharmacokinetic properties of other formulation components, or other factors.

Another approach to improve or mask the unpleasant taste of an inhaled drug may be to decrease the drug's solubility, e.g., drugs must dissolve to interact with taste receptors. Hence, to deliver solid forms of the drug may avoid the taste response and result in the desired improved taste affect. Non-limiting methods to decrease solubility of an ALK5 inhibitor compound solubility are described herein, for example, through the use in formulation of particular salt forms of ALK5 inhibitor compound, such as complexation with xinafoic acid, oleic acid, stearic acid and/or pamoic acid. Additional co-precipitating agents include dihydropyridines and a polymer such as polyvinyl pyrrolidone.

Moreover, taste-masking may be accomplished by creation of lipophilic vesicles. Additional coating or capping agents include dextrates (by non-limiting example cyclodextrins may include, 2-hydroxypropyl-beta-cyclodextrin, 2-hydroxypropyl-gamma-cyclodextrin, randomly methylated beta-cyclodextrin, dimethyl-alpha-cyclodextrin, dimethyl-beta-cyclodextrin, maltosyl-alpha-cyclodextrin, glucosyl-1-alpha-cyclodextrin, glucosyl-2-alpha-cyclodextrin, alpha-cyclodextrin, beta-cyclodextrin, gamma-cyclodextrin, and sulfobutylether-beta-cyclodextrin), modified celluloses such as ethyl cellulose, methyl cellulose, hydroxypropyl cellulose, hydroxypropyl methyl cellulose, polyalkylene glycols, polyalkylene oxides, sugars and sugar alcohols, waxes, shellacs, acrylics and mixtures thereof. By non-limiting example, other methods to deliver non-dissolved forms of an ALK5 inhibitor compound according to certain embodiments or, in other embodiments, non-dissolved forms of an ALK5 inhibitor compound, are to administer the drug alone or in a simple, non-solubility affecting formulation, such as a crystalline micronized, dry powder, spray-dried, and/or nanosuspension formulation.

In some embodiments, the taste-modifying agents are included in the ALK5 inhibitor compound formulation. These embodiments contemplate including in the formulation a taste-masking substance that is mixed with, coated onto or otherwise combined with the active medicament ALK5 inhibitor compound or salt thereof. Inclusion of one or more such agents in these formulations may also serve to improve the taste of additional pharmacologically active compounds that are included in the formulations in addition to the ALK5 inhibitor compound, e.g., a mucolytic agent. Non-limiting examples of such taste-modifying substances include acid phospholipids, lysophospholipid, tocopherol polyethyleneglycol succinate, and embonic acid (pamoate). Many of these agents can be used alone or in combination with an ALK5 inhibitor compound (or a salt thereof) or, in separate embodiments, ALK5 inhibitor compound for aerosol administration.

Methods to produce formulations that combine agents to reduce sputum viscosity during aerosol treatment with an ALK5 inhibitor compound include for example, N-acetylcysteine or Nacystelyn (NAL). These agents can be prepared in fixed combination or be administered in succession with aerosol ALK5 inhibitor compound therapy.

The most commonly prescribed agent is N-acetylcysteine (NAC), which depolymerizes mucus in vitro by breaking disulphide bridges between macromolecules. It is assumed that such reduction of sputum tenacity facilitates its removal from the respiratory tract. In addition, NAC may act as an oxygen radical scavenger. NAC can be taken either orally or by inhalation. Differences between these two methods of administration have not been formally studied. After oral administration, NAC is reduced to cysteine, a precursor of the antioxidant glutathione, in the liver and intestine. The antioxidant properties could be useful in preventing decline of lung function in cystic fibrosis (CF), chronic obstructive pulmonary disease (COPD) or pulmonary fibrotic diseases (e.g., idiopathic pulmonary fibrosis). Nebulized NAC is commonly prescribed to patients with CF, in particular in continental Europe, in order to improve expectoration of sputum by reducing its tenacity. The ultimate goal of this is to slow down the decline of lung function in CF.

L-lysine-N-acetylcysteine (ACC) or Nacystelyn (NAL) is a novel mucoactive agent possessing mucolytic, antioxidant, and anti-inflammatory properties. Chemically, it is a salt of ACC. This drug appears to present an activity superior to its parent molecule ACC because of a synergistic mucolytic activity of L-lysine and ACC. Furthermore, it's almost neutral pH (6.2) allows its administration in the lungs with a very low incidence of bronchospasm, which is not the case for the acidic ACC (pH 2.2). NAL is difficult to formulate in an inhaled form because the required lung dose is very high (approximately 2 mg) and the micronized drug is sticky and cohesive and it is thus problematic to produce a redispersible formulation. NAL was first developed as a chlorofluorocarbon (CFC) containing metered-dose inhaler (MDI) because this form was the easiest and the fastest to develop to begin the preclinical and the first clinical studies. NAL MDI delivered 2 mg per puff, from which approximately 10% was able to reach the lungs in healthy volunteers. One major inconvenience of this formulation was patient compliance because as many as 12 puffs were necessary to obtain the required dose. Furthermore, the progressive removal of CFC gases from medicinal products combined with the problems of coordination met in a large proportion of the patient population have led to the development of a new galenical form of NAL. A dry powder inhaler (DPI) formulation was chosen to resolve the problems of compliance with metered-dose inhalers and to combine it with an optimal, reproducible, and comfortable way to administer the drug to the widest possible patient population, including young children.

The dry powder inhaler formulation of NAL involved the use of a nonconventional lactose (usually reserved for direct compression of tablets), namely, a roller-dried (RD) anhydrous β-lactose. When tested in vitro with a monodose dry powder inhaler device, this powder formulation produces a fine particle fraction (FPF) of at least 30% of the nominal dose, namely three times higher than that with metered-dose inhalers. This approach may be used in combination with an ALK5 inhibitor compound for either co-administration or fixed combination therapy.

In addition to mucolytic activity, excessive neutrophil elastase activity within airways of cystic fibrosis (CF) patients results in progressive lung damage. Disruption of disulfide bonds on elastase by reducing agents may modify its enzymatic activity. Three naturally occurring dithiol reducing systems were examined for their effects on elastase activity: 1) Escherichia coli thioredoxin (Trx) system, 2) recombinant human thioredoxin (rhTrx) system, and 3) dihydrolipoic acid (DHLA). The Trx systems consisted of Trx, Trx reductase, and NADPH. As shown by spectrophotometric assay of elastase activity, the two Trx systems and DHLA inhibited purified human neutrophil elastase as well as the elastolytic activity present in the soluble phase (sol) of CF sputum. Removal of any of the three Trx system constituents prevented inhibition. Compared with the monothiols N-acetylcysteine and reduced glutathione, the dithiols displayed greater elastase inhibition. To streamline Trx as an investigational tool, a stable reduced form of rhTrx can be synthesized and used as a single component. Reduced rhTrx inhibits purified elastase and CF sputum sol elastase without NADPH or Trx reductase. The ability of Trx and DHLA to limit elastase activity combined with their mucolytic effects makes these compounds potential therapies for CF and could be combined with ALK-5 inhibitors for greater efficacy.

In addition, bundles of F-actin and DNA present in the sputum of cystic fibrosis (CF) patients but absent from normal airway fluid contribute to the altered viscoelastic properties of sputum that inhibit clearance of infected airway fluid and exacerbate the pathology of CF. Soluble multivalent anions have potential alone or in combination with other mucolytic agents to selectively dissociate the large bundles of charged biopolymers that form in CF sputum.

Hence, NAC, unfractionated heparin, reduced glutathione, dithiols, Trx, DHLA, other monothiols, DNAse, dornase alfa, hypertonic formulations (e.g., osmolalities greater than about 350 mOsmol/kg), multivalent anions such as polymeric aspartate or glutamate, glycosidases and other examples listed above can be combined with ALK5 inhibitor compounds and other mucolytic agents for aerosol administration to improve antifibrotic and/or anti-inflammatory activity through better distribution from reduced sputum viscosity, and improved clinical outcome through improved pulmonary function (from improved sputum mobility and mucociliary clearance) and decreased lung tissue damage from the immune inflammatory response.

In one embodiment, the compositions can be administered to the respiratory tract (including nasal and pulmonary) e.g., through a nebulizer, metered-dose inhalers, atomizer, mister, aerosol, dry powder inhaler, insufflator, liquid instillation or other suitable device or technique.

In some embodiments, aerosols intended for delivery to the nasal mucosa are provided for inhalation through the nose. For optimal delivery to the nasal cavities, inhaled particle sizes of about 5 to about 100 microns are useful, with particle sizes of about 10 to about 60 microns being preferred. For nasal delivery, a larger inhaled particle size may be desired to maximize impaction on the nasal mucosa and to minimize or prevent pulmonary deposition of the administered formulation. In some embodiments, aerosols intended for delivery to the lung are provided for inhalation through the nose or the mouth. For delivery to the lung, inhaled aerodynamic particle sizes of about less than 10 μm are useful (e.g., about 1 to about 10 microns). Inhaled particles may be defined as liquid droplets containing dissolved drug, liquid droplets containing suspended drug particles (in cases where the drug is insoluble in the suspending medium), dry particles of pure drug substance, drug substance incorporated with excipients, liposomes, emulsions, colloidal systems, coacervates, aggregates of drug nanoparticles, or dry particles of a diluent which contain embedded drug nanoparticles.

In some embodiments, compounds of Formula (I) disclosed herein intended for respiratory delivery (either systemic or local) can be administered as aqueous formulations, as non-aqueous solutions or suspensions, as suspensions or solutions in halogenated hydrocarbon propellants with or without alcohol, as a colloidal system, as emulsions, coacervates, or as dry powders. Aqueous formulations may be aerosolized by liquid nebulizers employing either hydraulic or ultrasonic atomization or by modified micropump systems (like the soft mist inhalers, the Aerodose® or the AERx® systems). Propellant-based systems may use suitable pressurized metered-dose inhalers (pMDIs). Dry powders may use dry powder inhaler devices (DPIs), which are capable of dispersing the drug substance effectively. A desired particle size and distribution may be obtained by choosing an appropriate device.

In some embodiments, a liquid solution for nebulized inhalation administration can comprises an ALK5 inhibitor compound, at a concentration from about 1 μg/mL to about 20 μg/mL in unit increments of about 0.1 μg/mL composition.

In some embodiments, a liquid solution for nebulized inhalation administration can comprises an ALK5 inhibitor compound, at a concentration from about 0.1 mg/mL to about 100 mg/mL in unit increments of about 0.01 mg/mL composition.

In some embodiments, each inhaled dose that is directly administered to the lungs of the mammal comprises from about 0.05 mL to about 10 mL of an aqueous solution of an ALK5 inhibitor compound in unit increments of about 0.01 mL.

In some embodiments, the osmolality is greater than about 50 mOsmol/kg composition in unit increments of about 1 mOsmol/kg.

In some embodiments, the pH is greater than about 3.0 in pH unit increments of about 0.1. By example, a pH of about 3, a pH of about 3.5, a pH of about 4, a pH of about 4.5, a pH of about 5, a pH of about 5.5, a pH of about 6, a pH of about 6.5, a pH of about 7, a pH of about 7.5, a pH of about 8, a pH of about 8.5 and a pH of about 9.

In some embodiments, the pH is balanced by the inclusion of an organic buffer selected from the group consisting of citric acid, citrate, malic acid, malate, pyridine, formic acid, formate, piperazine, succinic acid, succinate, histidine, maleate, bis-tris, pyrophosphate, phosphoric acid, phosphate, PIPES, ACES, MES, cacodylic acid, carbonic acid, carbonate, ADA (N-(2-Acetamido)-2-iminodiacetic acid).

In some embodiments, the ALK5 inhibitor compound solution contains a permeant ion concentration. In some embodiments, the permeant ion is selected from the group consisting of bromine, chloride, and lithium. In some embodiments, the permeant ion concentration is from about 10 mM to about 300 mM in 10 mM increments. By example, about 10 mM to 20 mM, 20 mM to 30 mM, 30 mM to about 40 mM, about 50 mM, about 60 mM, about 70 mM, about 80 mM, about 90 mM, about 100 mm, about 150 mM, about 200 mM, about 250 mM, and about 300 mM.

In some embodiments, the composition further comprises a taste masking agent. In some embodiments, the taste masking agent is selected from the group consisting of lactose, sucrose, dextrose, saccharin, aspartame, sucralose, ascorbate, multivalent cation and citrate. In some embodiments, the taste masking agent concentration is from 0.01 mM to about 50 mM in about 0.01 mM increments. By examples, about 0.01 mM, about 0.05 mM, about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 2 mM, about 3 mM, about 4 mM, about 5 mM, about 6 mM, about 7 mM, about 8 mM, about 9 mM, about 10 mM, about 15 mM, about 20 mM, about 25 mM, about 30 mM, about 35 mM, about 40 mM, about 45 mM, and about 50 mM.

In another embodiment, a pharmaceutical composition is provided that includes a simple liquid ALK5 inhibitor (of salt thereof) compound formulation with non-encapsulating water-soluble excipients having an osmolality from about 50 mOsmol/kg to about 6000 mOsmol/kg. In one embodiment, the osmolality is from about 50 mOsmol/kg to about 1000 mOsmol/kg. In one embodiment, the osmolality is from about 400 mOsmol/kg to about 5000 mOsmol/kg. In other embodiments the osmolality is from about 50, 100, 150, 200, 250, 300, 350, 400, 450, 500 mOsmol/kg to about 1000, 1100, 1200, 1300, 1400, 1500, 1600, 1700, 1800, 1900, 2000, 2200, 2400, 2600, 2800, 3000, 3200, 3400, 3600, 3800, 4000, 4200, 4400, 4600, 4800 m 5000, 5200, 5400, 5600, 5800 and 6000 mOsmol/kg.

In some embodiments, the inhaled doses are delivered, <4, <3, <2, <1 times a day, or less than daily. In some embodiments, the inhaled doses are delivered by nebulization using standard tidal breathing of continuous flow aerosol or breath actuated aerosol. In such embodiments of nebulized delivery, delivery times can be <10, <8, <6, <4, <2 and <1 minute. In some embodiments, the inhaled doses are delivered by inhalation of a dispersed dry powder aerosol using <10, <8, <6, <5, <4, <3, <2 or 1 breath of either a passive dispersion dry power inhaler or active dispersion dry powder inhaler. In some embodiments, the inhaled doses are delivered by inhalation of aerosol using <10, <8, <6, <5, <4, <3, <2 or 1 breath of a compressed gas metered dose inhaler with or without a spacer.

In another aspect, described herein is a method for the treatment of lung disease in a mammal comprising: administering a dose of an ALK5 inhibitor compound by inhalation to the mammal in need thereof, wherein the inhaled dose of an ALK5 inhibitor compound is administered with a nebulizer, a metered dose inhaler, or a dry powder inhaler. In some embodiments, the inhaled dose comprises an aqueous solution of an ALK5 inhibitor compound and the dose is administered with a liquid nebulizer. In some embodiments, each inhaled dose that is directly administered to the lungs of the mammal comprises from about 0.1 mL to about 6 mL of an aqueous solution of an ALK5 inhibitor compound, wherein the concentration of an ALK5 inhibitor compound in the aqueous solution is from about 0.1 mg/mL and about 60 mg/ml and the osmolality of the of the aqueous solution is from about 50 mOsmol/kg to about 6000 mOsmol/kg. In some embodiments, the aqueous solution of each inhaled dose further comprises: one or more additional ingredients selected from co-solvents, tonicity agents, sweeteners, surfactants, wetting agents, chelating agents, anti-oxidants, salts, and buffers. In some embodiments, the aqueous solution of each inhaled dose further comprises: a citrate buffer or phosphate buffer, and one or more salts selected from the group consisting of sodium chloride, magnesium chloride, sodium bromide, magnesium bromide, calcium chloride and calcium bromide. In some embodiments, the aqueous solution of each inhaled dose comprises: water; an ALK5 inhibitor compound at a concentration from about 0.1 mg/mL to about 20 mg/mL; one or more salts, wherein the total amount of the one or more salts is from about 0.01% to about 2.0% by weight of the weight of aqueous solution; and optionally a phosphate buffer that maintains the pH of the solution from about pH 5.0 to about pH 8.0, or citrate buffer than maintains the pH of the solution from about 4.0 to about 7.0. In some embodiments, the inhaled dose of an ALK5 inhibitor compound is administered on a continuous dosing schedule. In some embodiments, the lung disease is an Interstitial Lung Disease (ILD). In some embodiments, the Interstitial Lung Disease (ILD) is selected from the group consisting of: Idiopathic Pulmonary Fibrosis (IPF), scleroderma-associated interstitial lung disease (SSc-ILD), sarcoidosis, bronchiolitis obliterans, Langerhans cell histiocytosis (also called Eosinophilic granuloma or Histiocytosis X), chronic eosinophilic pneumonia, collagen vascular disease, granulomatous vasculitis, Goodpasture's syndrome, or pulmonary alveolar proteinosis (PAP). In some embodiments, the lung disease is idiopathic pulmonary fibrosis. In some embodiments, the lung disease is cystic fibrosis. In some embodiments, the method further comprises administration of one or more additional therapeutic agents to the mammal.

In one embodiment, a nebulizer is selected on the basis of allowing the formation of an aerosol of an ALK5 inhibitor compound disclosed herein having a median mass aerodynamic diameter (MMAD) predominantly between about 1 to about 5 microns. In one embodiment, the delivered amount of an ALK5 inhibitor compound provides a therapeutic effect for pulmonary pathology and/or extra-pulmonary, systemic, tissue or central nervous system distribution.

For aqueous and other non-pressurized liquid systems, a variety of nebulizers (including small volume nebulizers) are available to aerosolize the formulations. Compressor-driven nebulizers incorporate jet technology and use compressed air to generate the liquid aerosol. Such devices are commercially available from, for example, Healthdyne Technologies, Inc.; Invacare, Inc.; Mountain Medical Equipment, Inc.; Pari Respiratory, Inc.; Mada Medical, Inc.; Puritan-Bennet; Schuco, Inc., De Vilbiss Health Care, Inc.; and Hospitak, Inc. Ultrasonic nebulizers rely on mechanical energy in the form of vibration of a piezoelectric crystal to generate respirable liquid droplets and are commercially available from, for example, Omron Heathcare, Inc., Boehringer Ingelheim, and De Vilbiss Health Care, Inc. Vibrating mesh nebulizers rely upon either piezoelectric or mechanical pulses to respirable liquid droplets generate. Other examples of nebulizers for use with an ALK5 inhibitor compound are described in U.S. Pat. Nos. 4,268,460; 4,253,468; 4,046,146; 3,826,255; 4,649,911; 4,510,929; 4,624,251; 5,164,740; 5,586,550; 5,758,637; 6,644,304; 6,338,443; 5,906,202; 5,934,272; 5,960,792; 5,971,951; 6,070,575; 6,192,876; 6,230,706; 6,349,719; 6,367,470; 6,543,442; 6,584,971; 6,601,581; 4,263,907; 5,709,202; 5,823,179; 6,192,876; 6,644,304; 5,549,102; 6,083,922; 6,161,536; 6,264,922; 6,557,549; and 6,612,303 all of which are hereby incorporated by reference in their entirety.

Any known inhalation nebulizer suitable to provide delivery of a medicament as described herein may be used in the various embodiments and methods described herein. Such nebulizers include, e.g., jet nebulizers, ultrasonic nebulizers, pulsating membrane nebulizers, nebulizers with a vibrating mesh or plate with multiple apertures, and nebulizers comprising a vibration generator and an aqueous chamber (e.g., Pari eFlow®). Commercially available nebulizers suitable for use in the present disclosure can include the Aeroneb®, MicroAir®, Aeroneb® Pro, and Aeroneb® Go. Aeroneb® Solo, Aeroneb® Solo/Idehaler combination, Aeroneb® Solo or Go Idehaler-Pocket R combination, PARI LC-Plus®, PARI LC-Start, PARI Sprint R, eFlow and eFlow Rapid®, Pari Boy® N and Pari Duraneb® (PARI, GmbH), MicroAir®, (Omron Healthcare, Inc.), Halolite® (Profile Therapeutics Inc.), Respimat® (Boehringer Ingelheim), Aerodose® (Aerogen, Inc, Mountain View, Calif.), Omron Elite® (Omron Healthcare, Inc.), Omron Microair® (Omron Healthcare, Inc.), Mabismist II® (Mabis Healthcare, Inc.), Lumiscope® 6610, (The Lumiscope Company, Inc.), Airsep Mystique®, (AirSep Corporation), Acorn-1 and Acorn-II (Vital Signs, Inc.), Aquatower® (Medical Industries America), Ava-Neb® (Hudson Respiratory Care Incorporated), Cirrus® (Intersurgical Incorporated), Dart® (Professional Medical Products), Devilbiss® R Pulmo Aide (DeVilbiss Corp.), Downdraft R (Marquest), Fan Jet R (Marquest), MB-5 (Mefar), Misty Neb® (Baxter), Salter 8900 (Salter Labs), Sidestream R (Medic-Aid), Updraft-II® (Hudson Respiratory Care), Whisper Jet® (Marquest Medical Products), Aiolos R (Aiolos Medicnnsk Teknik), Inspiron® (Intertech Resources, Inc.), Optimist® (Unomedical Inc.), Prodomo®, Spira® (Respiratory Care Center), AERx® and AERx Essence™ (Aradigm), Respirgard II®, Sonik® LDI Nebulizer (Evit Labs), Swirler W Radioaerosol System (AMICI, Inc.), Maquet SUN 145 ultrasonic, Schill ultrasonic, compare and compare Elite from Omron, Monoghan AeroEclipse BAN, Transneb, De Vilbiss 800, AcrovectRx, Porta-Neb®, Freeway Freedom™, Sidestream, Ventstream and I-neb produced by Philips, Inc. By further non-limiting example, U.S. Pat. No. 6,196,219, is hereby incorporated by reference in its entirety.

Any of these and other known nebulizers suitable to provide delivery of an aqueous inhalation medicament as described herein may be used in the various embodiments and methods described herein. In some embodiments, the nebulizers are available from, e.g., Pari GmbH (Starnberg, Germany), De Vilbiss Healthcare (Heston, Middlesex, UK), Healthdyne, Vital Signs, Baxter, Allied Health Care, Invacare, Hudson, Omron, Bremed, AirSep. Luminscope, Medisana, Siemens, Aerogen, Mountain Medical, Aerosol Medical Ltd. (Colchester, Essex, UK), AFP Medical (Rugby, Warwickshire, UK), Bard Ltd. (Sunderland, UK), Carri-Med. Ltd. (Dorking, UK), Plaem Nuiva (Brescia, Italy), Henleys Medical Supplies (London, UK), Intersurgical (Berkshire, UK), Lifecare Hospital Supplies (Leies, UK), Medic-Aid Ltd. (West Sussex, UK), Medix Ltd. (Essex, UK), Sinclair Medical Ltd. (Surrey, UK), and many others.

Other nebulizers suitable for use in the methods and systems describe herein can include, but are not limited to, jet nebulizers (optionally sold with compressors), ultrasonic nebulizers, and others. Exemplary jet nebulizers for use herein can include Pari LC plus/ProNeb, Pari LC plus/ProNeb Turbo, Pari LCPlus/Dura Neb 1000 & 2000 Pari LC plus/Walkhaler, Pari LC plus/Pari Master, Pari LC star, Omron CompAir XL Portable Nebulizer System (NE-C18 and JetAir Disposable nebulizer), Omron compare Elite Compressor Nebulizer System (NE-C21 and Elite Air Reusable Nebulizer, Pari LC Plus or Pari LC Star nebulizer with Proneb Ultra compressor, Pulomo-aide, Pulmo-aide LT, Pulmo-aide traveler, Invacare Passport, Inspiration Healthdyne 626, Pulmo-Neb Traveler, De Vilbiss 646, Whisper Jet, AcornII, Misty-Neb, Allied aerosol, Schuco Home Care, Lexan Plasic Pocet Neb, SideStream H and Held Neb, Mobil Mist, Up-Draft, Up-DraftII, T Up-Draft, ISO-NEB. Ava-Neb, Micro Mist, and PulmoMate.

Exemplary ultrasonic nebulizers suitable to provide delivery of a medicament as described herein can include MicroAir, UltraAir, Siemens Ultra Nebulizer 145, CompAir, Pulmosonic, Scout, 5003 Ultrasonic Neb, 5110 Ultrasonic Neb, 5004 Desk Ultrasonic Nebulizer, Mystique Ultrasonic, Lumiscope's Ultrasonic Nebulizer, Medisana Ultrasonic Nebulizer, Microstat Ultrasonic Nebulizer, and Mabismist H and Held Ultrasonic Nebulizer. Other nebulizers for use herein include 5000 Electromagnetic Neb, 5001 Electromagnetic Neb 5002 Rotary Piston Neb, Lumineb I Piston Nebulizer 5500, Aeroneb Portable Nebulizer System, Aerodose Inhaler, and AcroEclipse Breath Actuated Nebulizer. Exemplary nebulizers comprising a vibrating mesh or plate with multiple apertures are described by R. Dhand in New Nebulizer Technology—Aerosol Generation by Using a Vibrating Mesh or Plate with Multiple Apertures, Long-Term Healthcare Strategies 2003. (July 2003), p. 1-4 and Respiratory Care, 47: 1406-1416 (2002), the entire disclosure of each of which is hereby incorporated by reference.

Additional nebulizers suitable for use in the presently described disclosure include nebulizers comprising a vibration generator and an aqueous chamber. Such nebulizers are sold commercially as, e.g., Pari eFlow, and are described in U.S. Pat. Nos. 6,962,151, 5,518,179, 5,261,601, and 5,152,456, each of which is specifically incorporated by reference herein.

Exemplary disclosure of compositions and methods for formulation delivery using nebulizers can be found in, e.g., US 2006/0276483, including descriptions of techniques, protocols and characterization of aerosolized mist delivery using a vibrating mesh nebulizer.

In one embodiment, a jet nebulizer is selected.

In one embodiment, an ultrasonic nebulizer is selected.

In one embodiment, a vibrating mesh nebulizer is selected.

In one embodiment, a vibrating mesh nebulizer is used to deliver an aerosol of an ALK5 inhibitor compound. A vibrating mesh nebulizer comprises a liquid storage container in fluid contact with a diaphragm and inhalation and exhalation valves. In one embodiment, about 1 to about 6 ml of an ALK5 inhibitor compound formulation is placed in the storage container and the aerosol generator is engaged producing atomized aerosol of particle sizes selectively between about 1 and about 5 microns. In one embodiment, about 1 to about 10 mL of an ALK5 inhibitor compound formulation is placed in the storage container and the aerosol generator is engaged producing atomized aerosol of particle sizes selectively between about 1 and about 5 microns. In one embodiment, about the volume of an ALK5 inhibitor compound formulation that is originally placed in the storage container and the aerosol generator is replaced to increase the administered dose size.

In one embodiment, a high efficiency liquid nebulizer is selected.

In some embodiments, the high efficiency liquid nebulizer achieves lung deposition of about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, or about 85%, based on the nominal dose of an ALK5 inhibitor compound administered to the mammal.

In some embodiments, the high efficiency liquid nebulizer provides a Geometric Standard Deviation (GSD) of emitted droplet size distribution of the solution administered with the high efficiency liquid nebulizer of about 1.0 μm to about 2.5 μm, about 1.2 μm to about 2.5 μm, about 1.3 μm to about 2.0 μm, at least about 1.4 μm to about 1.9 μm, at least about 1.5 μm to about 1.9 μm, about 1.5 μm, about 1.7 μm, or about 1.9 μm.

In some embodiments, the high efficiency liquid nebulizer provides a mass median aerodynamic diameter (MMAD) of droplet size of the solution emitted with the high efficiency liquid nebulizer of about 1 μm to about 5 μm, about 2 to about 4 μm, or about 2.5 to about 4.0 μm. In some embodiments, the high efficiency liquid nebulizer provides a volumetric mean diameter (VMD) 1 μm to about 5 μm, about 2 to about 4 μm, or about 2.5 to about 4.0 μm. In some embodiments, the high efficiency liquid nebulizer provides a mass median diameter (MMD) 1 μm to about 5 μm, about 2 to about 4 μm, or about 2.5 to about 4.0 μm.

In some embodiments, the high efficiency liquid nebulizer provides a fine particle fraction (FPF=%≤5 microns) of droplets emitted from the high efficiency nebulizer of about 60%, about 65%, about 70%, about 75%, about 80%, about 85%, or about 90%.

In some embodiments, the high efficiency liquid nebulizer provides an output rate of at least 0.1 mL/min, at least 0.2 mL/min, at least 0.3 mL/min, at least 0.4 mL/min, at least 0.5 mL/min, at least 0.6 mL/min, at least 0.8 mL/min, or at least 1.0 mL/min.

In some embodiments, the high efficiency liquid nebulizer (vi) delivers about 20%, about 25%, about 30%, about 35%, about 40%, about 45%, about 50%, about 55%, about 60%, about 65%, about 70%, about 75%, or about 80% of the fill volume to the mammal.

In some embodiments, the high efficiency liquid nebulizer provides an RDD of about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, or about 85%.

Additional features of a high efficiency liquid nebulizer with perforated membranes are disclosed in U.S. Pat. Nos. 6,962,151, 5,152,456, 5,261,601, and 5,518,179, U.S. Pat. No. 6,983,747, each of which is hereby incorporated by reference in its entirety. Other embodiments of the high efficiency liquid nebulizers contain oscillatable membranes. Features of these high efficiency liquid nebulizers are disclosed in U.S. Pat. Nos. 7,252,085; 7,059,320; 6,983,747, each of which is hereby incorporated by reference in its entirety.

Commercial high efficiency liquid nebulizers are available from: PARI (Germany) under the trade name eFlow R: Nektar Therapeutics (San Carlos, Calif.) under the trade names AeroNeb® Go and AeroNeb® Pro, and AeroNeb® Solo, Respironics (Murrysville, Calif.) under the trade names 1-Neb®, Omron (Bannockburn, Ill.) under the trade name Micro-Air®, Activaero (Germany) under the trade name Akita®, Aerogen (Galaway, Ireland) utilizing the OnQ® nebulizer technology, and Carefusion (San Diego, Calif.) under the trade name Airlife® Sidestream.

In one embodiment, a metered-dose inhaler is selected.

In some embodiments, the particle size of the drug substance in a metered-dose inhaler may be optimally chosen. In some embodiments, the particles of active ingredient have diameters of less than about 50 microns. In some embodiments, the particles have diameters of less than about 10 microns. In some embodiments, the particles have diameters of from about 1 micron to about 5 microns. In some embodiments, the particles have diameters of less than about 1 micron. In one advantageous embodiment, the particles have diameters of from about 2 microns to about 5 microns.

By non-limiting example, metered-dose inhalers, the ALK5 inhibitor compound disclosed herein are prepared in dosages from a formulation meeting the requirements of the metered-dose inhaler. The ALK5 inhibitor compound disclosed herein may be soluble in the propellant, soluble in the propellant plus a co-solvent (by non-limiting example ethanol), soluble in the propellant plus an additional moiety promoting increased solubility (by non-limiting example glycerol or phospholipid), or as a stable suspension or micronized, spray-dried or nanosuspension.

By non-limiting example, a metered-dose ALK5 inhibitor compound may be administered in the described respirable delivered dose in 10 or fewer inhalation breaths, in 8 or fewer inhalation breaths, in 6 or fewer inhalation breaths, in 4 or fewer inhalation breaths, or in 2 or fewer inhalation breaths.

The propellants for use with the metered-dose inhalers may be any propellants known in the art. Examples of propellants include chlorofluorocarbons (CFCs) such as dichlorodifluoromethane, trichlorofluoromethane, and dichlorotetrafluorocthanc; hydrofluoroalkanes (HFAs); and carbon dioxide. It may be advantageous to use HFAs instead of CFCs due to the environmental concerns associated with the use of CFCs. Examples of medicinal aerosol preparations containing HFAs are presented in U.S. Pat. Nos. 6,585,958; 2,868,691 and 3,014,844, all of which are hereby incorporated by reference in their entirety. In some embodiments, a co-solvent is mixed with the propellant to facilitate dissolution or suspension of the drug substance.

In some embodiments, the propellant and active ingredient are contained in separate containers, such as described in U.S. Pat. No. 4,534,345, which is hereby incorporated by reference in its entirety.

In some embodiments, the metered-dose inhaler used herein is activated by a patient pushing a lever, button, or other actuator. In other embodiments, the release of the aerosol is breath activated such that, after initially arming the unit, the active compound aerosol is released once the patient begins to inhale, such as described in U.S. Pat. Nos. 6,672,304; 5,404,871; 5,347,998; 5,284,133; 5,217,004; 5,119,806; 5,060,643; 4,664,107; 4,648,393; 3,789,843; 3,732,864; 3,636,949; 3,598,294; 3,565,070; 3,456,646; 3,456,645; and 3,456,644, each of which is hereby incorporated by reference in its entirety. Such a system enables more of the active compound to get into the lungs of the patient. Another mechanism to help a patient get adequate dosage with the active ingredient may include a valve mechanism that allows a patient to use more than one breath to inhale the drug, such as described in U.S. Pat. Nos. 4,470,412 and 5,385,140, both of which are hereby incorporated by reference in their entirety.

Additional examples of metered-dose inhalers known in the art and suitable for use herein include U.S. Pat. Nos. 6,435,177; 6,585,958; 5,642,730; 6,223,746; 4,955,371; 5,404,871; 5,364,838; and 6,523,536, all of which are hereby incorporated by reference in their entirety.

In one embodiment, a dry powder inhaler is selected.

By non-limiting example, a dry powder ALK5 inhibitor compound may be administered in the described respirable delivered dose in 10 or fewer inhalation breaths, in 8 or fewer inhalation breaths, in 6 or fewer inhalation breaths, in 4 or fewer inhalation breaths, or in 2 or fewer inhalation breaths.

In some embodiments, a dry powder inhaler is used to dispense the ALK5 inhibitor compound described herein. Dry powder inhalers contain the drug substance in fine dry particle form. Typically, inhalation by a patient causes the dry particles to form an aerosol cloud that is drawn into the patient's lungs. The fine dry drug particles may be produced by any technique known in the art. Some well-known techniques include use of a jet mill or other comminution equipment, precipitation from saturated or super saturated solutions, spray drying, in situ micronization (Hovione), or supercritical fluid methods. Typical powder formulations include production of spherical pellets or adhesive mixtures. In adhesive mixtures, the drug particles are attached to larger carrier particles, such as lactose monohydrate of size about 50 to about 100 microns in diameter. The larger carrier particles increase the aerodynamic forces on the carrier/drug agglomerates to improve aerosol formation. Turbulence and/or mechanical devices break the agglomerates into their constituent parts. The smaller drug particles are then drawn into the lungs while the larger carrier particles deposit in the mouth or throat. Some examples of adhesive mixtures are described in U.S. Pat. No. 5,478,578 and PCT Publication Nos. WO 95/11666, WO 87/05213, WO 96/23485, and WO 97/03649, all of which are incorporated by reference in their entirety. Additional excipients may also be included with the drug substance.

There are three common types of dry powder inhalers, all of which may be used with the ALK5 inhibitor compounds described herein. In a single-dose dry powder inhaler, a capsule containing one dose of dry drug substance/excipients is loaded into the inhaler. Upon activation, the capsule is breached, allowing the dry powder to be dispersed and inhaled using a dry powder inhaler. To dispense additional doses, the old capsule must be removed and an additional capsule loaded. Examples of single-dose dry powder inhalers are described in U.S. Pat. Nos. 3,807,400; 3,906,950; 3,991,761; and 4,013,075, all of which are hereby incorporated by reference in their entirety. In a multiple unit dose dry powder inhaler, a package containing multiple single dose compartments is provided. For example, the package may comprise a blister pack, where each blister compartment contains one dose. Each dose can be dispensed upon breach of a blister compartment. Any of several arrangements of compartments in the package can be used. For example, rotary or strip arrangements are common. Examples of multiple unit doses dry powder inhalers are described in EPO Patent Application Publication Nos. 0211595A2, 0455463A1, and 0467172A1, all of which are hereby incorporated by reference in their entirety. In a multi-dose dry powder inhaler, a single reservoir of dry powder is used. Mechanisms are provided that measure out single dose amounts from the reservoir to be aerosolized and inhaled, such as described in U.S. Pat. Nos. 5,829,434; 5,437,270; 2,587,215; 5,113,855; 5,840,279; 4,688,218; 4,667,668; 5,033,463; and 4,805,811 and PCT Publication No. WO 92/09322, all of which are hereby incorporated by reference in their entirety.

In some embodiments, auxiliary energy in addition to or other than a patient's inhalation may be provided to facilitate operation of a dry powder inhaler. For example, pressurized air may be provided to aid in powder de-agglomeration, such as described in U.S. Pat. Nos. 3,906,950; 5,113,855; 5,388,572; 6,029,662 and PCT Publication Nos. WO 93/12831, WO 90/07351, and WO 99/62495, all of which are hereby incorporated by reference in their entirety. Electrically driven impellers may also be provided, such as described in U.S. Pat. Nos. 3,948,264; 3,971,377; 4,147,166; 6,006,747 and PCT Publication No. WO 98/03217, all of which are hereby incorporated by reference in their entirety. Another mechanism is an electrically powered tapping piston, such as described in PCT Publication No. WO 90/13327, which is hereby incorporated by reference in its entirety. Other dry powder inhalers use a vibrator, such as described in U.S. Pat. Nos. 5,694,920 and 6,026,809, both of which are hereby incorporated by reference in their entirety. Finally, a scraper system may be employed, such as described in PCT Publication No. WO 93/24165, which is hereby incorporated by reference in its entirety.

Additional examples of dry powder inhalers for use herein are described in U.S. Pat. Nos. 4,811,731; 5,113,855; 5,840,279; 3,507,277; 3,669,113; 3,635,219; 3,991,761; 4,353,365; 4,889,144, 4,907,538; 5,829,434; 6,681,768; 6,561,186; 5,918,594; 6,003,512; 5,775,320; 5,740,794; and 6,626,173, all of which are hereby incorporated by reference in their entirety.

In some embodiments, a spacer or chamber may be used with any of the inhalers described herein to increase the amount of drug substance that gets absorbed by the patient, such as is described in U.S. Pat. Nos. 4,470,412; 4,790,305; 4,926,852; 5,012,803; 5,040,527; 5,024,467; 5,816,240; 5,027,806; and 6,026,807, all of which are hereby incorporated by reference in their entirety. For example, a spacer may delay the time from aerosol production to the time when the aerosol enters a patient's mouth. Such a delay may improve synchronization between the patient's inhalation and the aerosol production. A mask may also be incorporated for infants or other patients that have difficulty using the traditional mouthpiece, such as is described in U.S. Pat. Nos. 4,809,692; 4,832,015; 5,012,804; 5,427,089; 5,645,049; and 5,988,160, all of which are hereby incorporated by reference in their entirety.

Dry powder inhalers, which involve disaggregation and aerosolization of dry powder particles, normally rely upon a burst of inspired air that is drawn through the unit to deliver a drug dosage. Such devices are described in, for example, U.S. Pat. No. 4,807,814, which is directed to a pneumatic powder ejector having a suction stage and an injection stage: SU 628930 (Abstract), describing a hand-held powder disperser having an axial air flow tube: Fox et al., Powder and Bulk Engineering, pages 33-36 (March 1988), describing a venturi eductor having an axial air inlet tube upstream of a venturi restriction: EP 347 779, describing a hand-held powder disperser having a collapsible expansion chamber, and U.S. Pat. No. 5,785,049, directed to dry powder delivery devices for drugs.

Commercial examples of dry powder inhalers that can be used with the ALK5 inhibitor compound formulations described herein include the Acrolizer, Turohaler, Handihaler and Discus.

By non-limiting example, a nebulized ALK5 inhibitor compound may be administered in the described respirable delivered dose in less than about 20 min, less than about 15 min, less than about 10 min, less than about 7 min, less than about 5 min, less than about 3 min, or less than about 2 min.

The parameters used in nebulization, such as flow rate, mesh membrane size, aerosol inhalation chamber size, mask size and materials, valves, and power source may be varied as applicable to provide delivery of a medicament as described herein to maximize their use with different types and aqueous inhalation mixtures.

In some embodiments, the drug solution is formed prior to use of the nebulizer by a patient. In other embodiments, the drug is stored in the nebulizer in liquid form, which may include a suspension, solution, or the like. In other embodiments, the drug is store in the nebulizer in solid form. In this case, the solution is mixed upon activation of the nebulizer, such as described in U.S. Pat. No. 6,427,682 and PCT Publication No. WO 03/035030, both of which are hereby incorporated by reference in their entirety. In these nebulizers, the solid drug, optionally combined with excipients to form a solid composition, is stored in a separate compartment from a liquid solvent.

The liquid solvent is capable of dissolving the solid composition to form a liquid composition, which can be aerosolized and inhaled. Such capability is, among other factors, a function of the selected amount and, potentially, the composition of the liquid. To allow easy handling and reproducible dosing, the sterile aqueous liquid may be able to dissolve the solid composition within a short period of time, possibly under gentle shaking. In some embodiments, the final liquid is ready to use after no longer than about 30 seconds. In some cases, the solid composition is dissolved within about 20 seconds, and advantageously, within about 10 seconds.

As used herein, the terms “dissolve(d)”, “dissolving”, and “dissolution” refer to the disintegration of the solid composition and the release, i.e., the dissolution, of the active compound. As a result of dissolving the solid composition with the liquid solvent a liquid composition is formed in which the active compound is contained in the dissolved state. As used herein, the active compound is in the dissolved state when at least about 90 wt.-% are dissolved, and more preferably when at least about 95 wt.-% are dissolved.

With regard to basic separated-compartment nebulizer design, it primarily depends on the specific application whether it is more useful to accommodate the aqueous liquid and the solid composition within separate chambers of the same container or primary package, or whether they should be provided in separate containers. If separate containers are used, these are provided as a set within the same secondary package. The use of separate containers is especially preferred for nebulizers containing two or more doses of the active compound. There is no limit to the total number of containers provided in a multi-dose kit. In one embodiment, the solid composition is provided as unit doses within multiple containers or within multiple chambers of a container, whereas the liquid solvent is provided within one chamber or container. In this case, a favorable design provides the liquid in a metered-dose dispenser, which may consist of a glass or plastic bottle closed with a dispensing device, such as a mechanical pump for metering the liquid. For instance, one actuation of the pumping mechanism may dispense the exact amount of liquid for dissolving one dose unit of the solid composition.

In another embodiment for multiple-dose separated-compartment nebulizers, both the solid composition and the liquid solvent are provided as matched unit doses within multiple containers or within multiple chambers of a container. For instance, two-chambered containers can be used to hold one unit of the solid composition in one of the chambers and one unit of liquid in the other. As used herein, one unit is defined by the amount of drug present in the solid composition, which is one-unit dose. Such two-chambered containers may, however, also be used advantageously for nebulizers containing only one single drug dose.

In one embodiment of a separated-compartment nebulizer, a blister pack having two blisters is used, the blisters representing the chambers for containing the solid composition and the liquid solvent in matched quantities for preparing a dose unit of the final liquid composition. As used herein, a blister pack represents a thermoformed or pressure-formed primary packaging unit, most likely comprising a polymeric packaging material that optionally includes a metal foil, such as aluminum. The blister pack may be shaped to allow easy dispensing of the contents. For instance, one side of the pack may be tapered or have a tapered portion or region through which the content is dispensable into another vessel upon opening the blister pack at the tapered end. The tapered end may represent a tip.

In some embodiments, the two chambers of the blister pack are connected by a channel, the channel being adapted to direct fluid from the blister containing the liquid solvent to the blister containing the solid composition. During storage, the channel is closed with a seal. In this sense, a seal is any structure that prevents the liquid solvent from contacting the solid composition. The seal is preferably breakable or removable: breaking or removing the seal when the nebulizer is to be used will allow the liquid solvent to enter the other chamber and dissolve the solid composition. The dissolution process may be improved by shaking the blister pack. Thus, the final liquid composition for inhalation is obtained, the liquid being present in one or both of the chambers of the pack connected by the channel, depending on how the pack is held.

According to another embodiment, one of the chambers, preferably the one that is closer to the tapered portion of the blister pack communicates with a second channel, the channel extending from the chamber to a distal position of the tapered portion. During storage, this second channel does not communicate with the outside of the pack but is closed in an air-tight fashion. Optionally, the distal end of the second channel is closed by a breakable or removable cap or closure, which may e.g., be a twist-off cap, a break-off cap, or a cut-off cap.

In one embodiment, a vial or container having two compartments is used, the compartment representing the chambers for containing the solid composition and the liquid solvent in matched quantities for preparing a dose unit of the final liquid composition. The liquid composition and a second liquid solvent may be contained in matched quantities for preparing a dose unit of the final liquid composition (by non-limiting example in cases where two soluble excipients or the ALK5 inhibitor compound and excipient are unstable for storage, yet desired in the same mixture for administration.

In some embodiments, the two compartments are physically separated but in fluid communication such as when so the vial or container are connected by a channel or breakable barrier, the channel or breakable barrier being adapted to direct fluid between the two compartments to enable mixing prior to administration. During storage, the channel is closed with a seal or the breakable barrier intact. In this sense, a seal is any structure that prevents mixing of contents in the two compartments. The seal is preferably breakable or removable: breaking or removing the seal when the nebulizer is to be used will allow the liquid solvent to enter the other chamber and dissolve the solid composition or in the case of two liquids permit mixing. The dissolution or mixing process may be improved by shaking the container. Thus, the final liquid composition for inhalation is obtained, the liquid being present in one or both of the chambers of the pack connected by the channel or breakable barrier, depending on how the pack is held.

The solid composition itself can be provided in various different types of dosage forms, depending on the physicochemical properties of the drug, the desired dissolution rate, cost considerations, and other criteria. In one of the embodiments, the solid composition is a single unit. This implies that one-unit dose of the drug is comprised in a single, physically shaped solid form or article. In other words, the solid composition is coherent, which is in contrast to a multiple unit dosage form, in which the units are incoherent.

Examples of single units which may be used as dosage forms for the solid composition include tablets, such as compressed tablets, film-like units, foil-like units, wafers, lyophilized matrix units, and the like. In a preferred embodiment, the solid composition is a highly porous lyophilized form. Such lyophilizates, sometimes also called wafers or lyophilized tablets, are particularly useful for their rapid disintegration, which also enables the rapid dissolution of the active compound.

On the other hand, for some applications the solid composition may also be formed as a multiple unit dosage form as defined above. Examples of multiple units are powders, granules, microparticles, pellets, beads, lyophilized powders, and the like. In one embodiment, the solid composition is a lyophilized powder. Such a dispersed lyophilized system comprises a multitude of powder particles, and due to the lyophilization process used in the formation of the powder, each particle has an irregular, porous microstructure through which the powder is capable of absorbing water very rapidly, resulting in quick dissolution.

Another type of multiparticulate system which is also capable of achieving rapid drug dissolution is that of powders, granules, or pellets from water-soluble excipients which are coated with the drug, so that the drug is located at the outer surface of the individual particles. In this type of system, the water-soluble low molecular weight excipient is useful for preparing the cores of such coated particles, which can be subsequently coated with a coating composition comprising the drug and, preferably, one or more additional excipients, such as a binder, a pore former, a saccharide, a sugar alcohol, a film-forming polymer, a plasticizer, or other excipients used in pharmaceutical coating compositions.

In another embodiment, the solid composition resembles a coating layer that is coated on multiple units made of insoluble material. Examples of insoluble units include beads made of glass, polymers, metals, and mineral salts. Again, the desired effect is primarily rapid disintegration of the coating layer and quick drug dissolution, which is achieved by providing the solid composition in a physical form that has a particularly high surface-to-volume ratio. Typically, the coating composition will, in addition to the drug and the water-soluble low molecular weight excipient, comprise one or more excipients, such as those mentioned above for coating soluble particles, or any other excipient known to be useful in pharmaceutical coating compositions.

To achieve the desired effects, it may be useful to incorporate more than one water-soluble low molecular weight excipient into the solid composition. For instance, one excipient may be selected for its drug carrier and diluent capability, while another excipient may be selected to adjust the pH. If the final liquid composition needs to be buffered, two excipients that together form a buffer system may be selected.

In one embodiment, the liquid to be used in a separated-compartment nebulizer is an aqueous liquid, which is herein defined as a liquid whose major component is water. The liquid does not necessarily consist of water only: however, in one embodiment it is purified water. In another embodiment, the liquid contains other components or substances, preferably other liquid components, but possibly also dissolved solids. Liquid components other than water which may be useful include propylene glycol, glycerol, and polyethylene glycol. One of the reasons to incorporate a solid compound as a solute is that such a compound is desirable in the final liquid composition, but is incompatible with the solid composition or with a component thereof, such as the active ingredient.

Another desirable characteristic for the liquid solvent is that it is sterile. An aqueous liquid would be subject to the risk of considerable microbiological contamination and growth if no measures were taken to ensure sterility. In order to provide a substantially sterile liquid, an effective amount of an acceptable antimicrobial agent or preservative can be incorporated or the liquid can be sterilized prior to providing it and to seal it with an air-tight seal. In one embodiment, the liquid is a sterilized liquid free of preservatives and provided in an appropriate air-tight container. However, according to another embodiment in which the nebulizer contains multiple doses of the active compound, the liquid may be supplied in a multiple-dose container, such as a metered-dose dispenser, and may require a preservative to prevent microbial contamination after the first use.

It is to be noted that concentrations and dosage values may also vary depending on the specific compound and the severity of the condition to be alleviated. It is to be further understood that for any particular patient, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Also provided herein are kits. Typically, a kit includes one or more compounds or compositions as described herein. In certain embodiments, a kit can include one or more delivery systems, e.g., for delivering or administering a compound as provided herein, and directions for use of the kit (e.g., instructions for treating a patient). In another embodiment, the kit can include a compound or composition as described herein and a label that indicates that the contents are to be administered to a patient with an Interstitial Lung Disease (ILD). In another embodiment, the kit can include a compound or composition as described herein and a label that indicates that the contents are to be administered to a patient with one or more of idiopathic pulmonary fibrosis (IPF), familial pulmonary fibrosis (FPF), non-specific interstitial pneumonitis (NSIP), cryptogenic organizing pneumonia (COP), lymphocytic interstitial pneumonitis (LIP), respiratory bronchiolitis associated interstitial lung disease (RB-ILD), desquamative interstitial pneumonitis (DIP), usual interstitial pneumonia (UIP), giant cell interstitial pneumonia (GIP), hypersensitivity pneumonitis, pneumoconiosis, acute interstitial pneumonia (AIP). In another embodiment, the kit can include a compound or composition as described herein and a label that indicates that the contents are to be administered to a patient with cystic fibrosis.

### Methods of Treatment

The compounds and compositions described herein can be used as anti-fibrotic agents. In addition, the compounds can be used as inhibitors of one or more activin receptor-like kinases (ALKs). ALKs are part of the TGF-β superfamily which has been implicated into different physiological and pathological processes in a broad range of cell systems, including fibroblasts, immune, stem, endothelial, mural and tumor cells. Accordingly, the compounds and compositions described herein may also be used to alleviate any type of TGF-β-mediated condition. Examples of the TGF-β-mediated conditions include all types of pulmonary fibrotic diseases and lung cancer. In one embodiment, the TGF-β-mediated condition is idiopathic pulmonary fibrosis. Another example of TGF-β induced pathology is in cystic fibrosis.

In some embodiments, the disorder or disease is a lung disease.

In some embodiments, the disclosure provides a method of treating or ameliorating fibrosis in Interstitial Lung Disease (ILD)

In some embodiments, the Interstitial Lung Disease (ILD) is selected from the group consisting of: Idiopathic Pulmonary Fibrosis (IPF), Idiopathic Interstitial Pneumonia (IIP), scleroderma-associated interstitial lung disease (SSc-ILD), sarcoidosis, bronchiolitis obliterans, Langerhans cell histiocytosis (also called Eosinophilic granuloma or Histiocytosis X), chronic eosinophilic pneumonia, collagen vascular disease, granulomatous vasculitis, Goodpasture's syndrome, or pulmonary alveolar proteinosis (PAP).

In some embodiments, the disclosure provides a method of treating or ameliorating Idiopathic Interstitial Pneumonia (IIP) which is a form of pulmonary fibrosis and a subgroup of Interstitial Lung Disease (ILD).

In some embodiments, the Idiopathic Interstitial Pneumonia (IIP) is selected from the group consisting of: Idiopathic Pulmonary Fibrosis (IPF), Familial pulmonary fibrosis (FPF), Non-Specific Interstitial Pneumonitis (NSIP), Cryptogenic Organizing Pneumonia (COP), Lymphocytic Interstitial Pneumonitis (LIP), Respiratory Bronchiolitis associated Interstitial Lung Disease (RB-ILD), Desquamative Interstitial Pneumonitis (DIP), Usual Interstitial Pneumonia (UIP), Giant cell Interstitial Pneumonia (GIP), hypersensitivity pneumonitis (also called extrinsic allergic alveolitis), pneumoconiosis (also called an occupational interstitial lung disease), Acute Interstitial Pneumonia (AIP) also called Hamman-Rich Syndrome.

In some embodiments, the disclosure provides a method of treating or ameliorating diffuse alveolar damage as seen in acute respiratory distress syndrome (ARDS), transfusion related acute lung injury (TRALI), and acute interstitial pneumonia (AIP).

In some embodiments, the disclosure provides a method of treating or ameliorating pulmonary fibrosis associated with connective tissue and autoimmune diseases.

In some embodiments, the disclosure provides a method of treating or ameliorating drug-induced pulmonary fibrosis. In some embodiments, the drug-induced pulmonary fibrosis is caused by antibiotics (e.g., nitrofurantoin (Macrobid) and sulfasalazine (Azulfidine)), immunosuppressant drugs (e.g., methotrexate), drugs for heart conditions (e.g., amiodarone (Nexterone)), cancer chemotherapy drugs (e.g., cyclophosphamide), or biological agents used to treat cancer or immune disorders (e.g., adalimumab (Humira) or etanercept (Enbrel)).

In some embodiments, the disclosure provides a method of treating or ameliorating sarcoidosis. In some embodiments, the sarcoidosis can be selected from the group consisting of: annular sarcoidosis, erythrodermic sarcoidosis, iihthyosiform sarcoidosis, hypopigmented sarcoidosis, löfgren syndrome, Lupus pernio, morpheaform sarcoidosis, Mucosal sarcoidosis, neurosarcoidosis, papular sarcoid, scar sarcoid, subcutaneous sarcoidosis, systemic sarcoidosis, and ulcerative sarcoidosis.

In some embodiments, the disclosure provides a method of treating or ameliorating pulmonary fibrosis caused by an autoimmune disease (e.g., rheumatoid arthritis, Sjogren's, lupus erythematosus (also known as lupus), scleroderma, polymyositis, dermatomyositis, or vasculitis).

In some embodiments, the disclosure provides a method of treating or ameliorating pulmonary fibrosis caused by an infection (e.g., bacterial infections or viral infections (e.g., hepatitis C, adenovirus, herpes virus, and other viruses).

In some embodiments, the disclosure provides a method of treating or ameliorating pulmonary fibrosis caused by environmental exposure (e.g., asbestos fibers, grain dust, silica dust, certain gases, smoking, or radiation).

In some embodiments, the disclosure provides a method of treating or ameliorating pulmonary fibrosis caused by bronchiolitis (inflammation of the small airways (bronchioles)), alveolitis (inflammation of the air sacs (alveoli)), vasculitis (inflammation of the small blood vessels (capillaries)).

In some embodiments, the disclosure provides a method of treating cystic fibrosis.

In some embodiments, the disorder or disease is pulmonary fibrosis.

In some embodiments, the disorder or disease is idiopathic pulmonary fibrosis (IPF).

In some embodiments, the patient is a mammal.

In some embodiments, the mammal is a human.

In some embodiments, the disorder or disease is a fibrotic disorder, wherein the fibrotic disorder is selected from the group consisting of: skin fibrosis; scleroderma; progressive systemic fibrosis; lung fibrosis; muscle fibrosis; kidney fibrosis; glomerulosclerosis; glomerulonephritis; hypertrophic scar formation; uterine fibrosis; renal fibrosis; cirrhosis of the liver, liver fibrosis; adhesions; chronic obstructive pulmonary disease; fibrosis following myocardial infarction; pulmonary fibrosis; fibrosis and scarring associated with diffuse/interstitial lung disease; central nervous system fibrosis; fibrosis associated with proliferative vitreoretinopathy (PVR); restenosis; endometriosis; ischemic disease, and radiation fibrosis.

In an embodiment of the invention, the method comprises treating a patient with a pulmonary disease which is, or is associated with, a COVID infection, for example, treating a COVID patient with, or at risk of developing, lung fibrosis with a compound or a pharmaceutical composition of the invention.

### EXAMPLES

### Example 1. In Vitro Screen for ALK5 Kinase Inhibition

ALK5 kinase assay methods have been described in the art (Molecular Pharmacology (2002). 62(1), 58-64). Representative compounds are tested as follows for inhibition of ALK5 autophosphorylation activity and of the ALK5 phosphorylation of α-Casein.

Representative compounds are screened using the assay procedure for ALK5 kinase activity as described below.

In a 96 well filter-bottom plate (Millipore, #MSDV N6B 50), 58 μL Assay Buffer is added to reach well. Add 10 μL of Cold ATP mix in Assay Buffer, then 10 μL of a 1:10 dilution of α-Casein stock. Then add 2 μL of compound being tested (DMSO) at a 50× final concentration. Hot ATP mix (10 μL) is added, and the reaction is started with the addition of 10 μL of a 1:350 dilution of the ALK5 protein (2 nM final) in Assay Buffer with 0.05% BSA (Bovine Serum Albumin). The reaction is mixed for 5 minutes at room temperature, and then continued for 145 minutes at room temperature. The reaction is then stopped with the addition of 100 μL of ice-cold 20% TCA (trichloroacetic acid). The assay is then incubated for at least 1 hour at 4° C., and then the contents of each well are filtered by suction through the filter. The wells are washed three times with 200 μL ice-cold 10% TCA. The plate bottom is blotted before and after removing plastic sub-base, and dried overnight at room temperature. Add 30 μL of scintillation fluid, and count 1 minute per well on a Wallac Tri-Lux scintillation counter.

The IC₅₀ (nM) values reported in Table 2 below are the mean of two or more IC₅₀ values that were determined in one or more experiments.

Table 9 shows the activity of representative compounds of Formula I as provided herein.

TABLE 9

| Com- | IC₅₀ | Com- | IC₅₀ |
|---|---|---|---|
| pound | (nM) | pound | (nM) |
| 1 | 23.4 | 6 | 108 |
| 2 | 94.1 | 7 | 4.33 |
| 3 | 6.65 | 8 | 12.9 |
| 4 | 7.94 | 9 | 18.9 |
| 5 | 35.2 | | |

### Example 2. Human Cell Assay for ALK5 Inhibition

Assay methods that evaluate the inhibition of Collagen (1A2) expression have been described in the art (BMC Pulmonary Medicine (2018). 18(63), 1-13). Representative compounds are tested as follows for inhibition of Collagen (1A2) expression.

Human Fibroblast Cell Culture Stimulation: Human lung primary fibroblasts and A549 cell line are cultured in 6 well plates (Nunc Thermo Scientific) in the appropriated medium with 10% FBS; when cells reached 80% confluence the medium is changed at 2% FBS. Cells are stimulated with activated TGF-β (5 ng/ml) (R&D Systems Minneapolis, MN, USA), in the presence of the compound being tested (DMSO) at a range of concentrations (e.g., 10, 25, 50, 100, 250, and 500 nM). After the incubation period, cells and supernatants are collected, separated by centrifugation and frozen for further analysis.

RNA extraction and real-time polymerase chain reaction (RT-PCR): Total RNA is isolated from cultured cells, after treatment with TGF-β and the ALK5 inhibitor compounds following the protocol above, using the Qiagen RNeasy Mini Kit (Qiagen, Valencia, CA, USA) according to the manufacturer's recommendations. Samples are digested with DNase I (Qiagen) to remove contaminating genomic DNA. RNA concentration and purity of each sample were measured using UV spectrophotometry. A total of 1 μg of RNA was reverse-transcribed using the iScript cDNA synthesis kit (Bio Rad) with oligo deoxythymidine and random hexamer primers. The reverse transcriptase reaction proceeded in a total volume of 20 μL in a conventional thermal cycler (Bio-Rad) at 25° C. for 5 min, followed by 30 min at 42° C. and 5 min at 85° C. Reaction volumes of 20 μL were placed in 384-well optical reaction plates with adhesive covers (ABI Prism™ Applied Biosystems, Foster City, CA, USA) using SYBR Green PCR Master Mix and specific sequence primers (Sigma). Glyceraldehyde-3-phosphate dehydrogenase (GADPH) mRNA amplified from the same samples served as the internal control. Samples were heated to 95° C. for 10 min and then PCR amplification was achieved by 40 cycles at 95° C. for 15 sec and 60° C. for 1 min using the ABI Prism 7900 (Applied Biosystems). The relative expression of each targeted gene was normalized by subtracting the corresponding housekeeping genes (β-actin, GADPH, HPRT and RNA18s) threshold cycle (Ct) value using the comparative CT method (ΔΔCt methods).

Table 10 shows the activity of a representative compound of Formula I as provided herein.

TABLE 10

| Com- | IC₅₀ |
|---|---|
| pound | (nM) |
| 3 | 81 |

### Example 3. Intrinsic Clearance in Human Liver Microsomes

Assay methods that evaluate the intrinsic clearance (CLINT) in human liver microsomes (HLMs) have been described in the art (Drug Metab. Dispos., (2005), 33(9), 1304-1311 BMC Pulmonary Medicine (2018), 18(63), 1-13). Representative compounds are tested as follows for their intrinsic clearance (CLINT) in human liver microsomes as follows.

A 5 microsomal incubation cofactor solution was prepared with 100 mM potassium phosphate buffered to pH 7.4 (BD Biosciences, Woburn, Mass.) supplemented with 2 mM NADPH (Sigma-Aldrich, St. Louis, Mo.). 10 mM DMSO stocks of test compound are diluted and spiked into the cofactor solution to yield a 0.2 μM concentration (0.02% v/v DMSO). Aliquots of frozen human liver microsomes (Bioreclamation IVT, Baltimore Md.) are thawed and diluted into 100 mM potassium phosphate buffer to yield microsomal protein concentrations of 0.2 mg/mL. Cofactor/drug and microsomal solutions are pre-warmed separately for 4 minutes in a water bath held at 37° C. Incubations (n=1) are started by the combination of equal volumes of cofactor/drug solution with microsomal solution. The final concentration of test compound is 0.1 μM with a final 20 protein concentration of 0.1 mg/mL and final NADPH concentration of 1 mM. Samples are collected at times 0, 3, 8, 15, 30, and 45 minutes to monitor the disappearance of test compound. At each time point, 50 μL of incubation sample was removed and spiked into 25 μL of water plus 3% formic acid plus Internal Standard for reaction termination. Samples are then injected onto an AB Sciex API 4000 triple quadrupole mass spectrometer for quantitation by LC-MS/MS. Mobile Phase A consisting of HPLC grade water with 0.2% formic acid and Mobile Phase B consisting of 30 of HPLC grade acetonitrile with 0.2% formic acid with all samples run through a Thermo HyPURITY C18 50×2.1 mm column (Waltham, Mass.). HLM CLINT data is reported in units of mL/min/kg.

Table 11 shows the activity of a representative compound of Formula I as provided herein.

TABLE 11

| Com- | CL_{INT} |
|---|---|
| pound | (mL/min/kg) |
| 3 | 199 |

### Example 4. Efficacy Study in Lung Fibrosis Model

Compound 3 may be used to prevent and reverse lung fibrosis and improve lung function and exercise capacity. Male C57BL6/J mice (7-9 weeks old) from the Jackson Laboratory (Bar Harbor, ME), are used for efficacy studies. Bleomycin sulphate (MP Biomedicals, Solon, OH, USA) is dissolved in 0.9% saline and loaded into an IA-IC liquid MicroSprayer (PennCentury, Wyndmoor, PA, USA). Bleomycin (1-5 U/kg) in 50 ml is intratracheally sprayed into mice lightly anaesthetized with isoflurane (5% in 100%02). Control animals receives 50 ml of 0.9% saline. In efficacy studies, Compound 3 at an appropriate amount and frequency is dosed intranasally starting on 2 days (prophylactic) or 5 days after (therapeutic) bleomycin administration (2 U/kg). Animals are treated until the end of the study (day 21 post-bleomycin administration).

Lung Function and Exercise Capacity: Invasive lung function measurements are taken at specified time points following bleomycin. Mice are anesthetized with 2.5 mg sodium pentobarbital (Abbott Labs, IL), and the trachea cannulated. Animals are mechanically ventilated using a computer-controlled piston ventilator (flexiVent, SCIREQ Inc., Montreal, Canada: tidal volume=10 ml/kg, respiratory rate of 150 breaths/min, 3 cmH2O positive end-expiratory pressure). To test exercise capacity animals are placed on a treadmill at a 5% incline at a speed to 10 m/minute and the speed increased 1 m/minute every 4 minutes of the test until exhausted. Baseline exercise capacity for each mouse is assessed 2 days prior to either saline or bleomycin administration and then once weekly after administration at days 6, 12, and 19 post-instillation.

Bronchoalveolar Lavage Cell Assessment: Immediately following lung function measurements, mice are exsanguinated, bronchoalveolar lavage fluid (BALF) is collected, and total and differential cell counts are performed. Samples are centrifuged (3006 g) and the remaining BALF supernatants stored until analyzed for cytokine and chemokine levels using MSD multiplex kits (Gaithersburg, MD) and Millipore immunoassay kits (Billerica, MA). For the analysis of 4-hydroxyproline (HP), BALF samples are extracted with acetonitrile at volume of 1:6, respectively. After centrifugation, acetic acid (0.1% in water) is then added to the supernatants at volume of 1.5:1, respectively. Samples are mixed and centrifuged before injection for LC/MS/MS analysis. LC/MS/MS analysis is conducted by gradient HPLC with selective reaction monitoring (SRM). The calibration range was 40-2,000 ng/ml. The LC/MS/MS system used for the analysis consisted of an AB-Sciex API4000 with an electrospray source connected to an Agilent 1200 pump, a Waters 2777 autosampler, and an Agilent 1100 series column oven. Chromatographic analysis is conducted by HILIC HPLC with an Ascentis Express HILIC (3062.1 mm, 2.7 um) column. The SRM transition is m/z 132 to m/z 41.

Histology: After performing lung function measurements, whole lungs (that are not subject to bronchoalveolar lavage) are inflated under 25 cm H2O pressure with 10% neutral buffered formalin through the tracheal cannula and immersed in formalin for at least 24 h. After being processed into paraffin blocks, the lungs are sectioned (5 mm) and stained with either hematoxylin and cosin (H&E) or immunolabeled with an anti-collagen I antibody (rabbit polyclonal, Genetex Inc., Irvine, CA), to assess fibrotic changes in the lungs. To determine the fibrosis histopathology score for the lung of each mouse, the entire left and right longitudinal lung sections (at the level of tracheal bifurcation) are scored separately at 100× magnification and the scores were combined (total score range 0-8). Grading criteria are as follows: Grade 0=no apparent fibrosis; Grade 1=minimal fibrosis with rare foci of mostly interstitial alveolar septal fibrosis affecting less than 5% of the entire lung section; Grade 2=mild fibrosis characterized by multiple foci with thickening of alveolar septa by fibrosis and progressing to regions with fibrous deposition within the alveolar spaces with some damage to the alveoli, affecting 5-25% of the entire lung section; Grade 3=moderate fibrosis with multiple or single coalescing large areas of fibrosis effacing the alveoli with definitive damage to pulmonary architecture, affecting 25-50% of the entire lung section; Grade 4=marked fibrosis with severe distortion of pulmonary parenchyma by large contiguous fibrous areas, affecting 50-75% of the entire lung section. For collagen I IHC quantification, glass slides are scanned at 20× using a Zeiss Mirax scanner (Carl Zeiss Microimaging. Thornwood, NY), and digital slides generated were analyzed using Definiens Tissue Studio software (Definiens, Munich, Germany). Numerical results are expressed as percentage of positively labeled area (area of collagen I labeling/parenchymal tissue reference area). Additionally, staining for alpha-smooth muscle actin (Thermo Scientific, Freemont, CA) was done using a 3-step biotin-streptavidin-HRP detection method, and DAB (3,3-diaminobenzidine) as the detection chromogen.

Compound 3 treatment prevents and/or reverses bleomycin-induced lung fibrosis as evidenced by significant reduced HP content, improvements of fibrosis histopathology scores in the lung and reduced collage 1 assessed by IHC. Inhibition of lung fibrosis by administering Compound 3 leads to significantly improved lung function and increased exercise capacity.

### Example 5. Efficacy Study in a Syngeneic Lung Cancer Model

Compound 3 may be used to suppress tumor growth in the lungs of a syngeneic cancer model when administered alone or in combination with an immunotherapeutic agent. Six- to eight-week old BALB/c mice are used for in vivo efficacy studies in accordance with IACUC guidelines. Firefly luciferase-expressing CT-26 mouse colon carcinoma cells (luc-CT26, CSC-RR0237, Creative Biogene, Shirley, NY, USA) are grown at 37° C. in a 5% CO₂ humidified atmosphere in Dulbecco's Modified Eagle's Medium (D6429, Dominique Dutscher, Brumath, France) supplemented with 10% foetal bovine serum (500105NIDD, Dominique Dutscher), 0.2% glucose (19002-013, Gibco, Thermo Fisher Scientific, Hampton, NH, USA), 2 mM L-glutamine (X0550, Dominique Dutscher), 100 U/ml penicillin and 100 μg/ml streptomycin (15140155, Gibco, Thermo Fisher Scientific). Cell suspensions are prepared by enzymatic treatment with trypsin-EDTA (11560626, Thermo Fisher Scientific). Luc-CT26 cells (2×10⁵ cells/mouse) are injected intravenously into BALB/c mice to generate the cancer model in which tumor outgrowth in the lungs is observed. Following injection of CT26 cells and starting on day 6, the mice are monitored 3 times a week (i.e., day 6, day 8, day 11, day 13, etc.) using an IVIS spectrum imaging system (Caliper, PerkinElmer) after ip injection of D-luciferin. When the average bioluminescence intensity reaches 2×10⁶ p/s/cm²/sr across the treatment groups, the mice are treated with either (1) vehicle control, (2) Compound 3 in an appropriate amount and frequency dosed intranasally, (3) an immunotherapeutic agent, such as an anti-PD-1 antibody, at an appropriate amount and frequency dosed ip, or (4) Compound 3 and an immunotherapeutic agent, each at an appropriate amount and frequency. Body weight is measured twice weekly. Statistically significant reduction in bioluminescence intensity (BLI) is observed in the lungs for animals treated with Compound 3 alone, and increased efficacy, as measured by reduction in BLI, is observed in animals treated with a combination of Compound 3 and an immunotherapeutic agent.

1-2. (canceled)

3. A method for treating a patient suffering from a pulmonary disease which comprises administering a therapeutically effective amount of an ALK-5 (TGFβR¹) inhibitor having the structure of Formula I or a pharmaceutically acceptable salt thereof, wherein the compound or the pharmaceutically acceptable salt thereof is administered to the patient's lung so as to thereby treat the patient 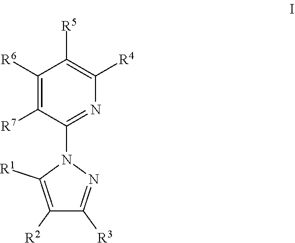
wherein
R¹ is selected from the group consisting of thieno[3,2-c]pyridinyl, thieno[3,2-b]pyridinyl, thieno[2,3-c]pyridinyl, and thieno[2,3-b]pyridinyl; wherein each may be optionally substituted with one to three substituents each independently selected from the group consisting of C₁-C₃-alkyl, —(C₁-C₃-alkyl)S(C₁-C₃-alkyl), —S(C₁-C₃-alkyl), —(C₁-C₃-alkyl)O(C₁-C₃-alkyl), —O(C₁-C₃-alkyl), —C(═O)O(C₁-C₃-alkyl), —CO₂H, —C(═O)NR⁸R⁹, halo, —CN, and —OH;
R² and R³ are independently selected from the group consisting of H, C₁-C₃-alkyl, —(C₁-C₃-alkyl)S(C₁-C₃-alkyl), —S(C₁-C₃-alkyl), —(C₁-C₃-alkyl)O(C₁-C₃-alkyl), —O(C₁-C₃-alkyl), —C(═O)O(C₁-C₃-alkyl), —CO₂H, —C(═O)NR¹⁰R¹¹, halo, —CN, —OH, and C₃-C₆-cycloalkyl;
alternatively, R² and R³ may be taken together to form a 5-6-membered heteroaryl, phenyl, a C₄-C₆-cycloalkyl, or a 4-6-membered heterocycloalkyl; wherein C₄-C₆-cycloalkyl and 4-6-membered heterocycloalkyl may be optionally substituted with one to three substituents independently selected from the group consisting of halo, —OH, oxo, and C₁-C₃ alkyl; wherein 5-6-membered heteroaryl and phenyl may be optionally substituted with one to three substituents independently selected from the group consisting of halo, —CN, —OH, —O(C₁-C₃ alkyl), and C₁-C₃ alkyl;
R⁴, R⁵, R⁶, and R⁷ are selected from the group consisting of H, C₃-cycloalkyl, C₁-C₃-alkyl, —(C₁-C₃-alkyl)S(C₁-C₃-alkyl), —S(C₁-C₃-alkyl), —(C₁-C₃-alkyl)O(C₁-C₃-alkyl), —O(C₁-C₃-alkyl), —C(═O)O(C₁-C₃-alkyl), —CO₂H, —C(═O)NR¹²R¹³, halo, —CN, —OH;
R⁸ and R⁹ are each independently selected from the group consisting of H, and —(C₁-C₃ alkyl) OH, C₁-C₃-alkyl, halo, and —O(C₁-C₃-alkyl);
R¹⁰ and R¹¹ are each independently selected from the group consisting of H and C₁-C₃ alkyl; and
R¹² and R¹³ are each independently selected from the group consisting of H, C₁-C₃ alkyl, halo, and —O(C₁-C₃-alkyl).

4-5. (canceled)

6. The method of claim 3, wherein the compound of Formula I is selected from the group consisting of: 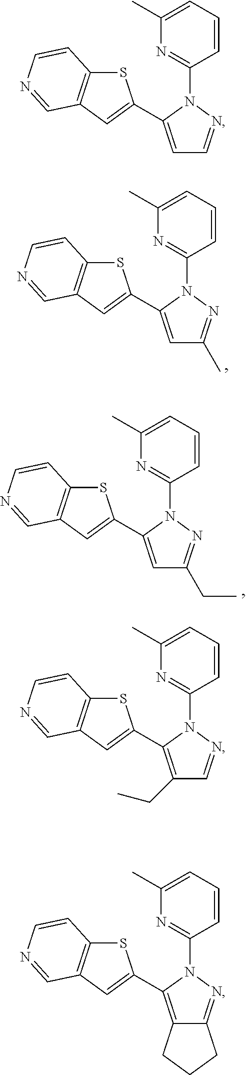 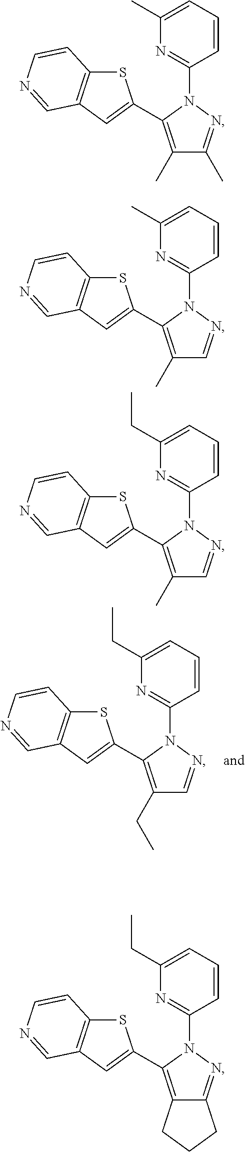
and pharmaceutically acceptable salts thereof.

7-10. (canceled)

11. The method according to claim 6, wherein the compound of Formula (I) is 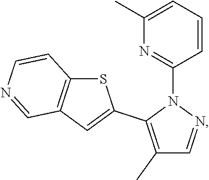 [SMILES: Cc1cccc(-n2ncc(C)c2-c2cc3cnccc3s2)n1]
or a pharmaceutically acceptable salt thereof.

12. The method according to claim 3, wherein each dose of the ALK5 inhibitor is administered by a device capable of delivering the effective amount of the ALK5 inhibitor and at least one pharmaceutically acceptable carrier to the lower airways via inhalation.

13. The method according to claim 12, wherein the device is selected from the group consisting of a nebulizer, a metered dose inhaler, or a dry powder inhaler.

14. The method according to claim 12, wherein the device is capable of delivering a liquid or suspension.

15. The method according to claim 12, wherein the effective amount comprises 0.1 mg to 100 mg of the ALK5 inhibitor.

16. The method according to claim 15, wherein the ALK5 inhibitor is formulated in a composition suitable for inhalation.

17-18. (canceled)

19. The method according to claim 16, wherein each inhaled dose comprises an aqueous solution and one or more additional ingredients selected from co-solvents, tonicity agents, sweeteners, surfactants, wetting agents, chelating agents, anti-oxidants, salts, and buffers.

20. The method according to claim 19, wherein the formulation of an ALK5 inhibitor is administered at least once a week.

21. The method according to claim 19, wherein the formulation of an ALK5 inhibitor is administered on a continuous daily dosing schedule.

22. The method according to claim 19, wherein the formulation of an ALK5 inhibitor is administered once a day, twice a day, or three times a day.

23-24. (canceled)

25. The method according to claim 3, wherein the pulmonary disease is interstitial lung disease.

26. The method according to claim 25, wherein the interstitial lung disease is selected from the group consisting of idiopathic pulmonary fibrosis (IPF), idiopathic interstitial pneumonia (IIP), scleroderma-associated interstitial lung disease (SSc-ILD), sarcoidosis, bronchiolitis obliterans, Langerhans cell histiocytosis (also called Eosinophilic granuloma or Histiocytosis X), chronic eosinophilic pneumonia, collagen vascular disease, granulomatous vasculitis, Goodpasture's syndrome, lung cancer, and pulmonary alveolar proteinosis (PAP).

27. The method according to claim 26, wherein the interstitial lung disease is idiopathic pulmonary fibrosis (IPF).

28. (canceled)

29. A pharmaceutical composition suitable for direct administration to the lower airways, comprising a therapeutically effective amount of an ALK5 inhibitor and a pharmaceutically acceptable carrier; wherein ALK5 inhibitor has the structure of Formula I, or pharmaceutically acceptable salt thereof: 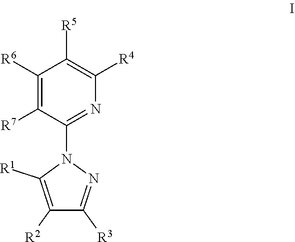
wherein
R¹ is selected from the group consisting of thieno[3,2-c]pyridinyl, thieno[3,2-b]pyridinyl, thieno[2,3-c]pyridinyl, and thieno[2,3-b]pyridinyl; wherein each may be optionally substituted with one to three substituents each independently selected from the group consisting of C₁-C₃-alkyl, —(C₁-C₃-alkyl)S(C₁-C₃-alkyl), —S(C₁-C₃-alkyl), —(C₁-C₃-alkyl)O(C₁-C₃-alkyl), —O(C₁-C₃-alkyl), —C(═O)O(C₁-C₃-alkyl), —CO₂H, C(═O)NR⁸R⁹, halo, —CN, and —OH;
R² and R³ are independently selected from the group consisting of H, C₁-C₃-alkyl, —(C₁-C₃-alkyl)S(C₁-C₃-alkyl), —S(C₁-C₃-alkyl), —(C₁-C₃-alkyl)O(C₁-C₃-alkyl), —O(C₁-C₃-alkyl), —C(═O)O(C₁-C₃-alkyl), —CO₂H, —C(═O)NR¹⁰R¹¹, halo, —CN, —OH, and C₃-C₅-cycloalkyl;
alternatively, R² and R³ may be taken together to form a 5-6-membered heteroaryl, phenyl, a C₄-C₆-cycloalkyl, or a 4-6-membered heterocycloalkyl; wherein C₄-C₆-cycloalkyl and 4-6-membered heterocycloalkyl may be optionally substituted with one to three substituents independently selected from the group consisting of halo, OH, oxo, and C₁-C₃ alkyl; wherein 5-6-membered heteroaryl and phenyl may be optionally substituted with one to three substituents independently selected from the group consisting of halo, —CN, —OH, —O(C₁-C₃ alkyl), and C₁-C₃ alkyl;
R⁴, R⁵, R⁶, and R⁷ are selected from the group consisting of H, C₃-cycloalkyl, C₁-C₃-alkyl, —(C₁-C₃-alkyl)S(C₁-C₃-alkyl), —S(C₁-C₃-alkyl), —(C₁-C₃-alkyl)O(C₁-C₃-alkyl), —O(C₁-C₃-alkyl), C(═O)O(C₁-C₃-alkyl), —CO₂H, —C(═O)NR¹³R¹³, halo, —CN, —OH;
R⁸ and R⁹ are each independently selected from the group consisting of H, and —(C₁-C₃ alkyl) OH, C₁-C₃-alkyl, halo, and —O(C₁-C₃-alkyl);
R¹⁰ and R¹¹ are each independently selected from the group consisting of H and C₁-C₃ alkyl; and
R¹² and R¹³ are each independently selected from the group consisting of H, C₁-C₃ alkyl, halo, and —O(C₁-C₃-alkyl).

30-33. (canceled)

34. The pharmaceutical composition according to claim 29, wherein the inhibitor has the structure of Formula I selected from the group consisting of: 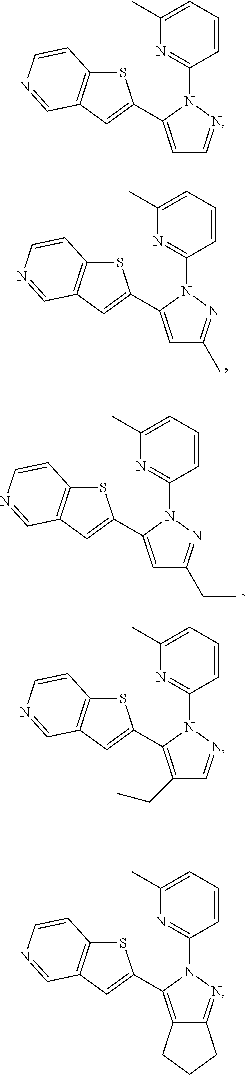 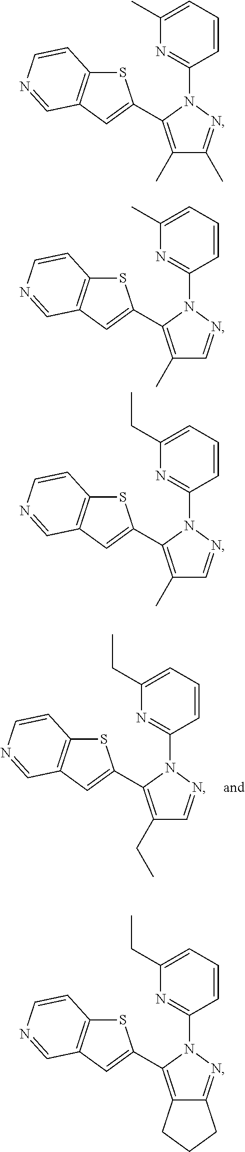
and pharmaceutically acceptable salts thereof.

35-38. (canceled)

39. The pharmaceutical composition according to claim 34, wherein the inhibitor has the structure: 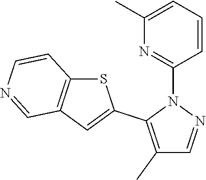 [SMILES: Cc1cccc(-n2ncc(C)c2-c2cc3cnccc3s2)n1]
or a pharmaceutically acceptable salt thereof.

40. The pharmaceutical composition according to claim 29, wherein the therapeutically effective amount comprises 0.1 mg to 100 mg of the ALK5 inhibitor.

41. The pharmaceutical composition according to claim 40, wherein the ALK5 inhibitor is present in a carrier suitable for inhalation.

42. The pharmaceutical composition according to claim 41, wherein the carrier is an aqueous solution of an ALK5 inhibitor.

43. (canceled)

44. The pharmaceutical composition according to claim 42, wherein the aqueous solution of each inhaled dose further comprises: one or more additional ingredients selected from co-solvents, tonicity agents, sweeteners, surfactants, wetting agents, chelating agents, anti-oxidants, salts, and buffers.

45-50. (canceled)
